(19) United States
(12) Patent Application Publication
(10) Pub. No.: US 2024/0390497 A1
(43) Pub. Date: Nov. 28, 2024

(54) COMPOSITIONS OF CHIMERIC ANTIGEN RECEPTOR (CAR) SIGNALING MOLECULES AND USES THEREOF

(71) Applicant: The Board of Trustees of the Leland Stanford Junior University, Stanford, CA (US)

(72) Inventors: Rebecca RICHARDS, Stanford, CA (US); Crystal L. MACKALL, Stanford, CA (US); Ravindra MAJETI, Stanford, CA (US)

(21) Appl. No.: 18/691,685

(22) PCT Filed: Sep. 14, 2022
(86) PCT No.: PCT/US2022/076439

§ 371 Date: Mar. 13, 2024

Related U.S. Application Data
(60) Provisional application No. 63/244,636, filed on Sep. 15, 2021.

(51) Int. Cl. A61K 39/00; A61P 35/00; C07K 14/725; C07K 16/28

(52) U.S. Cl. CPC A61K 39/464429; A61K 39/4611; A61K 39/4631; A61P 35/00; C07K 14/7051; C07K 16/2896; C07K 2317/24

(57) ABSTRACT

The present disclosure generally relates to, inter alia, recombinant cells capable of expressing polypeptides, e.g., chimeric antigen receptors (CARs) that can be used for logic gating (e.g., NOT-gating) to specifically target and eliminate some types of cells (e.g., cancer cells). The disclosure also provides compositions and methods useful for producing such recombinant cells or polypeptides, as well as methods for the detection and treatment of diseases, such as cancer.

### CROSS-REFERENCE TO RELATED APPLICATIONS

This application claims the benefit of priority to U.S. Provisional Patent Application Ser. No. 63/244,636, filed on Sep. 15, 2021. The disclosure of the above referenced application is herein expressly incorporated by reference to its entirety, including any drawings.

### STATEMENT REGARDING FEDERALLY SPONSORED R&D

The invention was made with Government support under contract CA232568 awarded by the National Institutes of Health. The Government has certain rights in the invention.

### INCORPORATION OF THE SEQUENCE LISTING

The material in the accompanying Sequence Listing is hereby incorporated by reference into this application. The accompanying Sequence Listing XML file, named 078430-537001WO_SequenceListing.XXML, was created on Sep. 5, 2022, and is 15.1 KB in size.

### FIELD

The present disclosure relates generally to the fields of oncology and immuno-therapeutics, and particularly relates to compositions of recombinant cells capable of expressing polypeptides, e.g., chimeric antigen receptors (CARs) that can be used for logic gating (e.g., NOT-gates) to specifically target and eliminate some types of cells (e.g., cancer cells), with mitigated “on-target, off-tumor” side effect to other non-cancer cells. The disclosure also provides compositions and methods useful for producing such recombinant cells or polypeptides, as well as methods for the detection and treatment of conditions, such as diseases (e.g., cancer).

### BACKGROUND

Acute myeloid leukemia (AML) is the most prevalent acute leukemia in the United States, accounting for more than 11,000 deaths each year with a 5-year overall survival rate of less than 30% (Siegel et al., CA Cancer J Clin. 2020; 70(1):7-30). Molecular analysis has accelerated understanding of the genetic diversity of AML and resulted in the approval of numerous molecularly targeted agents, including a Bcl-2 inhibitor (venetoclax), epigenetic modifiers of IDH1/2 (enasidenib, ivosidenib), FLT3 inhibitors (midostaurin, gilteritinib), and a Hedgehog inhibitor (glasdegib) (Lai et al., Recent drug approvals for acute myeloid leukemia. J Hematol Oncol. 2019; 12(1):100). Gemtuzumab ozogamycin, a CD33 directed antibody drug conjugate, is the only immunotherapy agent currently approved for use in AML (Amadori et al., Gemtuzumab Ozogamicin Versus Best Supportive Care in Older Patients With Newly Diagnosed Acute Myeloid Leukemia Unsuitable for Intensive Chemotherapy: Results of the Randomized Phase III EORTC-GIMNEMA AML-19 Trial. J Clin Oncol. 2016; 34(9):972-979; Castaigne et al., Effect of gemtuzumab ozogamicin on survival of adult patients with de-novo acute myeloid leukaemia (ALFA-0701): a randomised, open-label, phase 3 study. Lancet. 2012; 379(9825):1508-1516). Despite these emerging therapies, relapse rates, morbidity, and overall mortality in AML patients remain high and novel therapies are needed to improve survival and decrease toxicity.

CD19 CAR T cell therapy has greatly expanded therapeutic options for patients with chemorefractory B-ALL and DLBCL (Gardner et al., Intent-to-treat leukemia remission by CD19 CAR T cells of defined formulation and dose in children and young adults. Blood 2017; 129(25):3322-3331; Lee et al., T cells expressing CD19 chimeric antigen receptors for acute lymphoblastic leukaemia in children and young adults: a phase 1 dose-escalation trial. Lancet. 2015; 385(9967):517-528; Maude et al., Chimeric antigen receptor T cells for sustained remissions in leukemia. N Engl J Med 2014; 371(16):1507-1517; Locke et al., Long-term safety and activity of axicabtagene ciloleucel in refractory large B-cell lymphoma (ZUMA-1): a single-arm, multicentre, phase 1-2 trial. Lancet Oncol. 2019; 20(1):31-42; Schuster et al. Tisagenlecleucel in Adult Relapsed or Refractory Diffuse Large B-Cell Lymphoma. N Engl J Med 2019; 380(1):45-56; Porter et al., Chimeric antigen receptor-modified T cells in chronic lymphoid leukemia. N Engl J Med 2011; 365(8):725-733; Brentjens et al., CD19-targeted T cells rapidly induce molecular remissions in adults with chemotherapy-refractory acute lymphoblastic leukemia. Sci Transl Med 2013; 5(177):177ra138; Turtle et al., CD19 CAR-T cells of defined CD4+:CD8+ composition in adult B cell ALL patients. J Clin Invest. 2016; 126(6):2123-2138). These results have prompted intense interest in developing CAR T cells for other hematologic malignancies. However, one major concern for CAR T cell therapy is its “on-target, off-tumor” side effect when the CAR T cells attack and eliminate non-cancer cells expressing the same target antigen designed for the CAR T cells. There are not many identified cell surface antigens only expressed on cancer cells but not on non-cancer cells.

Consequently, there remains a need for more specific CAR T therapies to overcome these obstacles to extend the reach of these therapeutics to more diseases and to treat more patients.

### SUMMARY

The present disclosure relates generally to the development of immuno-therapeutics, including recombinant cells capable of expressing polypeptides such as inhibitory chimeric antigen receptors (iCARs), either alone or in combination as Boolean logic NOT gates, as well as pharmaceutical compositions containing the cells or the polypeptides for use in treating various conditions, such as diseases (e.g., cancers). As described in greater detail below, the iCARs, with or without various modifications (e.g., modifications to its intracellular signaling domain, a.k.a., cytosolic signaling region), have been found to recognize a target antigen expressed by a non-cancer cell (e.g., a normal or healthy cell) and inhibit activation of other activating CARs (aCARs) expressed by a recombinant cell (e.g., a T cell), thus reducing activation of the recombinant cell against the non-cancer cell. In some embodiments, the intracellular signaling domains of these iCAR molecules have an immunoreceptor tyrosine-based inhibitory motif (ITIM), which enables the iCAR molecules to inhibit activation of other activating CAR (aCAR) molecules [e.g., those having an immune receptor tyrosine based activation motif (ITAM), such as CD3zeta (CD3ζ)]. Furthermore, experimental results described herein have demonstrated that combinations of these iCAR and aCAR molecules similar to Boolean logic gates (e.g. NOT-gates) integrate signals based on the presence of multiple antigens on different cells, which would drastically increase their safety to non-cancer cells expressing both antigens recognizable by the iCAR and aCAR molecules. Domain swap and mutations have been used to engineer iCAR molecules to test specificity and/or potency.

In one aspect, the present disclosure provides a composition containing a recombinant cell expressing a first chimeric antigen receptor (CAR) polypeptide and a second chimeric antigen receptor (CAR) polypeptide. In some embodiments, the first CAR polypeptide expressed by the recombinant cell of the composition comprises:
1a) a first extracellular ligand-binding domain having a binding affinity for a first ligand;
1b) a first transmembrane domain; and
1c) a first intracellular signaling domain.
   In some embodiments, the second CAR polypeptide expressed by the recombinant cell of the composition comprises:
2a) a second extracellular ligand-binding domain having a binding affinity for a second ligand;
2b) a second transmembrane domain; and
2c) a second intracellular signaling domain.
   In some embodiments, binding of the first ligand to the first extracellular ligand-binding domain activates the first CAR polypeptide, and leads to activation of the recombinant cell to inhibit growth or kill a first cell (e.g., a cancer cell) expressing a sufficient amount of the first ligand but not the second ligand. In some embodiments, binding of the second ligand to the second extracellular ligand-binding domain activates the second CAR polypeptide, and leads to reduction of the activation of the first CAR polypeptide, thereby reducing the capability of the recombinant cell to inhibit growth or kill a second cell expressing a sufficient amount of the second ligand. In some embodiments, the second cell expresses a sufficient amount of both the first ligand and the second ligand.

In one aspect, the present disclosure provides a composition containing a recombinant cell expressing a first chimeric antigen receptor (CAR) polypeptide and a second chimeric antigen receptor (CAR) polypeptide, wherein the first CAR polypeptide comprises:
1a) a first extracellular ligand-binding domain having a binding affinity for a first ligand;
1b) a first transmembrane domain; and
1c) a first intracellular signaling domain,
   wherein the second CAR polypeptide comprises:
2a) a second extracellular ligand-binding domain having a binding affinity for a second ligand;
2b) a second transmembrane domain; and
2c) a second intracellular signaling domain,
   wherein binding of the first ligand to the first extracellular ligand-binding domain activates the first CAR polypeptide, and leads to activation of the recombinant cell to inhibit growth or kill a first cancer cell expressing a sufficient amount of the first ligand but not the second ligand, and wherein binding of the second ligand to the second extracellular ligand-binding domain activates the second CAR polypeptide, and leads to reduction of the activation of the first CAR polypeptide, thereby reducing the capability of the recombinant cell to inhibit growth or kill a second cell expressing a sufficient amount of the second ligand. In some embodiments, the second cell expresses a sufficient amount of both the first ligand and the second ligand.

In some embodiments, the first ligand is expressed on the cell membrane of the first cell (e.g., a cancer cell). In some embodiments, the second ligand is expressed on the cell membrane of the second cell. In some embodiments, the first ligand is expressed on the cell membrane of the first cell (e.g., a cancer cell) and the second ligand is expressed on the cell membrane of the second cell. In some embodiments, the second ligand is not expressed or expressed only in an insufficient amount on the cell membrane of the first cell.

In some embodiments, the first ligand comprises CD93 or HER2. Other exemplary first ligands may include antigens described herein, such as antigens specifically expressed on cancer cells or cells targeted to kill/inhibit growth.

Exemplary second ligands may include antigens expressed by a second cell (e.g., a non-cancer cell) different from a first cell (e.g., a cancer cell). As in non-limiting examples, a NOT-gate CAR T system described herein, e.g., cells expressing an activating CAR (aCAR) and an inhibitory CAR (iCAR) constructs, may different a first cell expressing a first antigen as the binding ligand for the aCAR construct from a second cell expressing a second antigen as the binding ligand for the iCAR construct. As a result, T cells expressing such NOT-gate system may be activated and kill or inhibit the growth of the first cell (e.g., a cancer cell), but not the second cell (e.g., a non-cancer cell), due to the inhibition of T cell activation by the iCAR upon binding to the second antigen/ligand. In some embodiments, the second antigen is not expressed by the first cell (e.g., a cancer cell), or expressed only in a low level (e.g., insufficiently to bind and activate the inhibitory CAR construct to inhibit the activation of the activating CAR construct and thus the T cells).

In some embodiments, at least one of the first extracellular ligand-binding domain and the second extracellular ligand-binding domain comprises a ligand-binding domain of an antibody, an antigen-binding fragment, an antibody mimetic, or a receptor. In some embodiments, the antibody or the antigen-binding fragment is selected from the group consisting of a monoclonal antibody, an antigen-binding fragment (Fab), a nanobody, a diabody, a triabody, a minibody, an F(ab′)2 fragment, an F(ab)v fragment, a single chain variable fragment (scFv), a single domain antibody (sdAb), a VH domain, a VL domain, an Fv fragment, a VNAR domain, and a VHH domain.

In some embodiments, the first extracellular ligand-binding domain comprises an antibody, an antigen-binding fragment, or an antibody mimetic capable of binding to CD93. In some embodiments, the first extracellular ligand-binding domain comprises a variable heavy (VH) domain containing CDR H1, CDR H2 and CDR H3 sequences in a VH framework. In some embodiments, at least one of the CDR H1, CDR H2 and CDR H3 sequences comprises an amino acid sequence having at least 70%, 75%, 80%, 85%, 90%, 91%, 92%, 93%, 94%, 95%, 96%, 97%, 98%, 99%, or 100% identity to SEQ ID NOs: 3, 4, or 5. In some embodiments, the variable heavy (VH) domain comprises an amino acid sequence having at least 70%, 75%, 80%, 85%, 90%, 91%, 92%, 93%, 94%, 95%, 96%, 97%, 98%, 99%, or 100% identity to SEQ ID NO: 1 or 2. In some embodiments, the first extracellular ligand-binding domain further comprises a variable light (VL) domain containing CDR L1, CDR L2 and CDR L3 sequences in a VL framework. In some embodiments, at least one of the CDR L1, CDR L2 and CDR L3 sequences comprises an amino acid sequence having at least 70%, 75%, 80%, 85%, 90%, 91%, 92%, 93%, 94%, 95%, 96%, 97%, 98%, 99%, or 100% identity to SEQ ID NOs: 8, 9, or 10. In some embodiments, the variable light (VL) domain comprises an amino acid sequence having at least 70%, 75%, 80%, 85%, 90%, 91%, 92%, 93%, 94%, 95%, 96%, 97%, 98%, 99%, or 100% identity to SEQ ID NO: 6 or 7.

In some embodiments, the VH and the VL domains are conjugated through a linker, such as a peptide linker. Exemplary linkers may be found in Chen et al., Fusion Protein Linkers: Property, Design and Functionality. Adv Drug Deliv Rev. 2013 October; 65(10):1357-69, expressly incorporated herein by reference to its entity. In some embodiments, the linker comprises a (G₄S)ₙ linker. The length (“n”) of such GS linker can be optimized to achieve appropriate separation of the functional domains, or to maintain necessary inter-domain interactions. In some embodiments, the linker contains a (G₄S)₃ linker.

In some embodiments, the first extracellular ligand-binding domain comprises an amino acid sequence having at least 70%, 75%, 80%, 85%, 90%, 91%, 92%, 93%, 94%, 95%, 96%, 97%, 98%, 99%, or 100% identity to any one of SEQ ID NOs: 12-15.

In some embodiments, at least one of the first transmembrane domain and the second transmembrane domain comprises a CD8 or CD28 transmembrane (TM) domain or a transmembrane domain derived from a CD8 or CD28 TM domain. Other exemplary TM domains may include those known to a skilled artisan and/or described herein.

In some embodiments, the first intracellular signaling domain comprises an intracellular signaling domain of or derived from CD3ζ.

In some embodiments, the second intracellular signaling domain comprises an immunoreceptor tyrosine-based inhibitory motif (ITIM) or ITIM-like domain.

In some embodiments, the second intracellular signaling domain comprises an intracellular signaling domain of or derived from PD-1, CTLA-4, LAIR1 (LIR-1), TIGIT, Sig 2, Sig 5, Sig 6, Sig 10, BTLA, LAG3, CD300a, or SIRPα.

In some embodiments, at least one of the first CAR polypeptide and the second CAR polypeptide further comprises a hinge domain and/or a costimulatory domain. In some embodiments, the hinge domain is or is derived from an IgG4 hinge domain, a CD28 hinge domain, or a CD8 hinge domain. Other exemplary hinge domains and/or costimulatory domains may include those known to a skilled artisan and/or described herein.

In some embodiments, the first cancer cell described herein is derived from a cancer selected from Acute Myeloid Leukemia (AML), Myelodysplastic Syndrome, Myeloproliferative Neoplasms, and Acute Myeloid Leukemia with MLL rearrangements. In some embodiments, the cancer is acute myeloid leukemia (AML).

In some embodiments, the second cell described herein is a non-cancer cell. In some embodiments, the non-cancer cell is an endothelial cell.

In some embodiments, the recombinant cell described herein is an immune cell. In some embodiments, the immune cell is a T cell, a tumor-infiltrating lymphocyte (TIL), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell.

In some embodiments, activation of the first CAR polypeptide in response to the first antigen enhances cell proliferation, differentiation, cytokine production and/or cytotoxicity of the recombinant cell.

In some embodiments, activation of the second CAR polypeptide in response to the second antigen reduces cell proliferation, differentiation, cytokine production and/or cytotoxicity of the recombinant cell.

In some embodiments, the composition described herein is capable of:
i) inhibiting growth and/or killing the first cancer cell but not the second cell; and/or
ii) treating a cancer in a subject in need of.

In another aspect, the present description provides a composition containing an inhibitory chimeric antigen receptor (iCAR) polypeptide capable of reducing activation of an activating chimeric antigen receptor (aCAR) polypeptide. In some embodiments, the aCAR polypeptide comprises:
1a) a first extracellular ligand-binding domain having a binding affinity for a first ligand;
1b) a first transmembrane domain; and
1c) a first intracellular signaling domain.

In some embodiments, the iCAR polypeptide comprises:
2a) a second extracellular ligand-binding domain having a binding affinity for a second ligand;
2b) a second transmembrane domain; and
2c) a second intracellular signaling domain.

In another aspect, the present description provides a composition containing an inhibitory chimeric antigen receptor (iCAR) polypeptide capable of reducing activation of an activating chimeric antigen receptor (aCAR) polypeptide,

wherein the aCAR polypeptide comprises:
1a) a first extracellular ligand-binding domain having a binding affinity for a first ligand;
1b) a first transmembrane domain; and
1c) a first intracellular signaling domain,
   wherein the iCAR polypeptide comprises:
2a) a second extracellular ligand-binding domain having a binding affinity for a second ligand;
2b) a second transmembrane domain; and
2c) a second intracellular signaling domain,
   wherein binding of the first ligand to the first extracellular ligand-binding domain activates the aCAR polypeptide, and leads to activation of a recombinant cell expressing both the aCAR and the iCAR polypeptides to inhibit growth or kill a first cancer cell expressing a sufficient amount of the first ligand but not the second ligand, and
   wherein binding of the second ligand to the second extracellular ligand-binding domain activates the iCAR polypeptide, and leads to reduction of the activation of the aCAR polypeptide, thereby reducing the capability of the recombinant cell to inhibit growth or kill a second cell expressing a sufficient amount of the second ligand. In some embodiments, the second cell expresses a sufficient amount of both the first and the second ligands.

In some embodiments, the first ligand is expressed on the cell membrane of the first cell (e.g., a cancer cell). In some embodiments, the second ligand is expressed on the cell membrane of the second cell. In some embodiments, the first ligand is expressed on cell membrane of the first cell (e.g., cancer cell) and the second ligand is expressed on cell membrane of the second cell. In some embodiments, the second ligand is not expressed or expressed only in an insufficient amount on the cell membrane of the first cell.

In some embodiments, the first ligand comprises CD93 or HER2. Other exemplary first ligands may include antigens described herein or known to a skilled artisan, such as antigens specifically expressed on cancer cells or cells targeted to kill/inhibit growth.

Exemplary second ligands may include antigens expressed by a second cell (e.g., a non-cancer cell) different from a first cell (e.g., a cancer cell). As in non-limiting examples, a NOT-gate CAR T system described herein, e.g., cells expressing an activating CAR (aCAR) and an inhibitory CAR (iCAR) constructs, may different a first cell expressing a first antigen as the binding ligand for the aCAR construct from a second cell expressing a second antigen as the binding ligand for the iCAR construct. As a result, T cells expressing such NOT-gate system may be activated and kill or inhibit the growth of the first cell (e.g., a cancer cell), but not the second cell (e.g., a non-cancer cell), due to the inhibition of T cell activation by the iCAR upon binding to the second antigen/ligand. In some embodiments, the second antigen is not expressed by the first cell (e.g., a cancer cell), or expressed only in a low level (e.g., insufficiently to bind and activate the inhibitory CAR construct to inhibit the activation of the activating CAR construct and thus the T cells).

In some embodiments, at least one of the first extracellular ligand-binding domain and the second extracellular ligand-binding domain comprises a ligand-binding domain of an antibody, an antigen-binding fragment, an antibody mimetic, or a receptor.

In some embodiments, the antibody or the antigen-binding fragment is selected from the group consisting of a monoclonal antibody, an antigen-binding fragment (Fab), a nanobody, a diabody, a triabody, a minibody, an F(ab′)2 fragment, an F(ab)v fragment, a single chain variable fragment (scFv), a single domain antibody (sdAb), a VH domain, a VL domain, an Fv fragment, a VNAR domain, and a VHH domain.

In some embodiments, the first extracellular ligand-binding domain comprises an antibody, an antigen-binding fragment, or an antibody mimetic capable of binding to CD93. In some embodiments, the first extracellular ligand-binding domain comprises a variable heavy (VH) domain containing CDR H1, CDR H2 and CDR H3 sequences in a VH framework.

In some embodiments, at least one of the CDR H1, CDR H2 and CDR H3 sequences comprises an amino acid sequence having at least 70%, 75%, 80%, 85%, 90%, 91%, 92%, 93%, 94%, 95%, 96%, 97%, 98%, 99%, or 100% identity to SEQ ID NOs: 3, 4, or 5. In some embodiments, the variable heavy (VH) domain comprises an amino acid sequence having at least 70%, 75%, 80%, 85%, 90%, 91%, 92%, 93%, 94%, 95%, 96%, 97%, 98%, 99%, or 100% identity to SEQ ID NO: 1 or 2.

In some embodiments, the first extracellular ligand-binding domain further comprises a variable light (VL) domain containing CDR L1, CDR L2 and CDR L3 sequences in a VL framework. In some embodiments, at least one of the CDR L1, CDR L2 and CDR L3 sequences comprises an amino acid sequence having at least 70%, 75%, 80%, 85%, 90%, 91%, 92%, 93%, 94%, 95%, 96%, 97%, 98%, 99%, or 100% identity to SEQ ID NOs: 8, 9, or 10. In some embodiments, the variable light (VL) domain comprises an amino acid sequence having at least 70%, 75%, 80%, 85%, 90%, 91%, 92%, 93%, 94%, 95%, 96%, 97%, 98%, 99%, or 100% identity to SEQ ID NO: 6 or 7.

In some embodiments, the VH and the VL domains are conjugated through a linker, such as a peptide linker. Exemplary linkers may be found in Chen et al., Fusion Protein Linkers: Property, Design and Functionality. Adv Drug Deliv Rev. 2013 October; 65(10):1357-69, expressly incorporated herein by reference to its entity. In some embodiments, the linker comprises a (G₄S)ₙ linker. The length (“n”) of such GS linker can be optimized to achieve appropriate separation of the functional domains, or to maintain necessary inter-domain interactions. In some embodiments, the linker contains a (G₄S)₃ linker.

In some embodiments, the first extracellular ligand-binding domain comprises an amino acid sequence having at least 70%, 75%, 80%, 85%, 90%, 91%, 92%, 93%, 94%, 95%, 96%, 97%, 98%, 99%, or 100% identity to any one of SEQ ID NOs: 12-15.

In some embodiments, at least one of the first transmembrane domain and the second transmembrane domain comprises a CD8 or CD28 transmembrane (TM) domain, or a TM domain derived from a CD8 or CD28 TM domain. Other exemplary TM domains may include those known to a skilled artisan and/or described herein.

In some embodiments, the first intracellular signaling domain comprises an intracellular signaling domain of or derived from CD3ζ.

In some embodiments, the second intracellular signaling domain comprises an immunoreceptor tyrosine-based inhibitory motif (ITIM) or ITIM-like domain.

In some embodiments, the second intracellular signaling domain comprises an intracellular signaling domain of or derived from PD-1, CTLA-4, LAIR1 (LIR-1), TIGIT, Sig 2, Sig 5, Sig 6, Sig 10, BTLA, LAG3, CD300a, or SIRPα.

In some embodiments, at least one of the aCAR polypeptide and the iCAR polypeptide further comprises a hinge domain and/or a costimulatory domain. In some embodiments, the hinge domain is or is derived from an IgG4 hinge domain, a CD28 hinge domain, or a CD8 hinge domain. Other exemplary hinge domains and/or costimulatory domains may include those known to a skilled artisan and/or described herein.

In some embodiments, the first cancer cell is derived from a cancer selected from Acute Myeloid Leukemia (AML), Myelodysplastic Syndrome, Myeloproliferative Neoplasms, or Acute Myeloid Leukemia with MLL rearrangements. In some embodiments, the cancer is acute myeloid leukemia (AML).

In some embodiments, the second cell is a non-cancer cell. In some embodiments, the non-cancer cell is an endothelial cell.

In some embodiments, the recombinant cell is an immune cell. In some embodiments, the immune cell is a T cell, a tumor-infiltrating lymphocyte (TIL), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell.

In some embodiments, activation of the aCAR polypeptide in response to the first antigen enhances cell proliferation, differentiation, cytokine production and/or cytotoxicity of the recombinant cell.

In some embodiments, activation of the iCAR polypeptide in response to the second antigen reduces cell proliferation, differentiation, cytokine production and/or cytotoxicity of the recombinant cell.

In some embodiments, the composition described herein is capable of:
i) inhibiting growth and/or killing the first cancer cell but not the second cell; and/or
ii) treating a cancer in a subject in need of.

In another aspect, the present disclosure provides a system containing an activating chimeric antigen receptor (aCAR) polypeptide and an inhibitory chimeric antigen receptor (iCAR) polypeptide. In some embodiment, the aCAR polypeptide in the system comprises:
1a) a first extracellular ligand-binding domain having a binding affinity for a first ligand;
1b) a first transmembrane domain; and
1c) a first intracellular signaling domain.

In some embodiments, the iCAR polypeptide in the system comprises:
2a) a second extracellular ligand-binding domain having a binding affinity for a second ligand;
2b) a second transmembrane domain; and
2c) a second intracellular signaling domain.

In some embodiments, binding of the first ligand to the first extracellular ligand-binding domain activates the aCAR polypeptide, and leads to activation of a recombinant cell expressing both the aCAR and the iCAR polypeptides to inhibit growth or kill a first cancer cell expressing a sufficient amount of the first ligand but not the second ligand. In some embodiments, binding of the second ligand to the second extracellular ligand-binding domain activates the iCAR polypeptide, and leads to reduction of the activation of the aCAR polypeptide, thereby reducing the capability of the recombinant cell to inhibit growth or kill a second cell expressing a sufficient amount of the second ligand. In some embodiments, the second cell expresses a sufficient amount of both the first and the second ligands.

In another aspect, the present disclosure provides a system containing an activating chimeric antigen receptor (aCAR) polypeptide and an inhibitory chimeric antigen receptor (iCAR) polypeptide, wherein the aCAR polypeptide comprises:
1a) a first extracellular ligand-binding domain having a binding affinity for a first ligand;
1b) a first transmembrane domain; and
1c) a first intracellular signaling domain,
   wherein the iCAR polypeptide comprises:
2a) a second extracellular ligand-binding domain having a binding affinity for a second ligand;
2b) a second transmembrane domain; and
2c) a second intracellular signaling domain,
   wherein binding of the first ligand to the first extracellular ligand-binding domain activates the aCAR polypeptide, and leads to activation of a recombinant cell expressing both the aCAR and the iCAR polypeptides to inhibit growth or kill a first cancer cell expressing a sufficient amount of the first ligand but not the second ligand, and wherein binding of the second ligand to the second extracellular ligand-binding domain activates the iCAR polypeptide, and leads to reduction of the activation of the aCAR polypeptide, thereby reducing the capability of the recombinant cell to inhibit growth or kill a second cell expressing a sufficient amount of the second ligand. In some embodiments, the second cell expresses a sufficient amount of both the first and the second ligands.

In another aspect, the present disclosure provides a composition containing a polynucleotide encoding the iCAR polypeptide described herein.

In another aspect, the present disclosure provides a composition containing a first polynucleotide encoding the aCAR polypeptide and a second polynucleotide encoding the iCAR polypeptide, as described herein.

In another aspect, the present disclosure provides a composition containing an expression vector containing a polynucleotide encoding the iCAR polypeptide described herein.

In another aspect, the present disclosure provides a composition containing a first expression vector containing a first polynucleotide encoding the aCAR polypeptide described herein, and a second expression vector expressing a second polynucleotide encoding the iCAR polypeptide described herein.

In another aspect, the present disclosure provides a composition containing an expression vector containing a first polynucleotide encoding the aCAR polypeptide described herein and a second polynucleotide encoding the iCAR polypeptide described herein.

In another aspect, the present disclosure provides a method for production of the recombinant cell or the CAR polypeptide(s), as described herein, in a cell, containing introducing a polynucleotide encoding the CAR polypeptide(s) into the cell, and inducing expression of the CAR polypeptide(s) under a condition.

In another aspect, the present disclosure provides a method for selectively activating the recombinant cell described herein, containing contacting the recombinant cell with the first ligand but not the second ligand.

In another aspect, the present disclosure provides a method for selectively inhibiting growth and/or killing of a cancer cell, containing contacting the recombinant cell described herein with the cancer cell, wherein the cancer cell expresses a sufficient amount of the first ligand but not the second ligand.

In another aspect, the present disclosure provides a method for selectively inhibiting growth and/or killing of a first cancer cell but not a second cell, containing contacting the recombinant cell described herein with the first cancer cell and the second cell, wherein the first cancer cell expresses a sufficient amount of the first ligand but not the second ligand, and the second cell expresses a sufficient amount of the second ligand. In some embodiments, the second cell expresses a sufficient amount of both the first ligand and the second ligand.

In another aspect, the present disclosure provides a method of treating a cancer in a subject in need of, containing administering to the subject a pharmaceutically effective amount of the recombinant cell described herein, wherein the first cancer cell is derived from the cancer.

The foregoing summary is illustrative only and is not intended to be in any way limiting. In addition to the illustrative embodiments and features described herein, further aspects, embodiments, objects and features of the disclosure can become fully apparent from the drawings and the detailed description and the claims.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIGS. 1A-1E are sets of graphs illustrating that CD93 is expressed on AML and mature myeloid cells. FIGS. 1A and 1B show that CD93 expression on primary AML was evaluated by flow cytometry, both in MLL-rearranged (MLLr) (FIG. 1A) or non-HLLr (FIG. 1B) primary patient samples. Cells were stained with CD93 (blue, the right-side peak in each panel) or isotype (red, the left-side peak in each panel). Representative samples are shown. FIG. 1C show that MLLr AML (n=11), non-MLLr AML (n=14) or HSCs (n=2) were stained for CD93 expression. The y-axis is represented as fold-MFI change compared to isotype control. FIG. 1D shows bulk CD34+ selected cells that were either unstained or stained with CD93 and a panel of antibodies to delineate the listed progenitor populations (see Example 1 for antibody specifications and FIGS. 3M-3N for gating strategy). FIG. 1E contains a set of flow cytometry data showing CD93 expression on mature hematopoietic cells, evaluated by staining PBMCs with CD93 and lineage markers including CD19 (B cells), CD3 (T cells), CD235a (red blood cells), CD41a (platelets), CD15 (neutrophils), and CD14 (monocytes).

FIGS. 2A-2O are sets of graphs illustrating that CD93-CAR T cells exert anti-leukemic effects against AML in vitro. FIG. 2A is a bar chart showing that humanized (“HuF11-G1” and “HuF11-G1 TM”) and chimeric F11 (“ChF11-G1”) monoclonal antibodies bind human CD93 with similar affinity. Human CD93/Fc fusion protein was coated in a 96-well plate and different concentrations of the antibodies as indicated were added. HRP-conjugated anti-human Kappa antibody was used as a secondary antibody. CD93 binding activity was measured by reading signals at OD490 mm. FIG. 2B is a cartoon showing the structure of CD93-28z and CD93-BBz CARs. From the N- to C-terminus, CD93 CARs were designed with VL and VH of the CD93 specific F11 scFv connected via a (G₄S)₃ linker, CD28 or CD8a TM, CD28 or 4-1BB costimulatory domain, and CD3ζ activation domain. FIG. 2C shows a CAR T cell production schema. Primary human T cells were activated on day 0 with CD3/28 beads and cultured with IL-2 (100 IU/ml). Cells were transduced with retroviral vectors containing CAR constructs on consecutive days 3 and 4, and activation beads were removed on day 5. Cells were fed with new media every 2-3 days, analyzed for transduction efficiency and phenotype on day 10-11, and processed for downstream experiments. FIG. 2D is a bar graph showing comparing transduction efficiency for each of aCAR molecule and control (“Mock”), measured by flow cytometry on day 10 after T cell activation. FIG. 2E is a liner chart showing the total cell counts for expanded CD93-CAR T cells over 10 days of culture, in representative experiments. FIG. 2F is a set of graphs comparing expression levels of markers of T cell activation/exhaustion, including LAG-3, PD-1, TIM-3, and CD39, measured by flow cytometry on day 10 after T cell activation, prior to downstream experiments. Summary data from n=4 donors are shown and gated on CAR positivity for CD93-28z or CD93-BBz, and on live singlets for mock-transduced CAR T cells (unpaired t test). FIG. 2G is a graph showing CD93 expression on AML cell lines, measured by flow cytometry after staining with biotinylated F11 antibody followed by streptavidin-APC. FIG. 2H is a graph showing schematic of F11-based CD93 CARs. Light (V_{L}) and heavy (V_{H}) chains were grafted onto a backbone of CD28 hinge/TM, CD28 costimulatory domain, and CD3ζ activation domain (28z) or CD8a hinge/TM, 4-1BB costimulatory domain, and CD3ζ activation domain (BBz). F11 28z L-H and F11 BBz L-H constructs were named CD93-28z and CD93-BBz, respectively. FIG. 2I is a graph showing CD93 CAR expression after transduction of primary T cells with F11-based CARs, analyzed with a CD93-Fc biotinylated protein followed by secondary antibody staining. FIG. 2J is a line graph showing expansion of CAR T cells transduced with mock (the top trace) or F11-based CARs (from the second-to-top trace to the bottom trace: F11 BBz H-L, F11 BBz L-H, F11 28z H-L, and F11 28z L-H), when co-cultured with THP-1 cells stably expressing GFP at a 1:1 E:T ratio. GFP expression was measured in an Incucyte™ for 72 h. FIG. 2K is a set of bar charts comparing cytokine (IFNγ: the left panel; IL-2: the right panel) production levels by mock-transduced or F11-based CAR T cells incubated at a 1:1 E:T ratio with various AML cells for 24 h, using ELISA to measure the supernatants. The bars for treatment with each AML cell line represent, from left to right, the treatment with T cells transduced with mock, F11 28z L-H, F11 28z H-L, F11 BBz L-H, and F11 BBz H-L, respectively. FIG. 2L is a set of graphs showing summary data of a similar experiment as in FIG. 2K from 3 donors (unpaired t test). The bars for treatment with each AML cell line represent, from left to right, the treatment with T cells transduced with mock, CD93-28z, and CD93-BBz, respectively. FIG. 2M is a set of graphs showing that IL-2 production of CD93 CAR T cells correlates directly to MFI of CD93 on various AML cell lines, normalized to HEL-2, an AML cell line with low CD93 expression that does not induce cytokine production; p=0.0289 for CD93-28z and p=0.0650 for CD93-BBz (linear regression analysis). FIG. 2N is a set of graphs showing IFNγ production of CD93 CAR T cells correlated with MFI of CD93 on various AML cell lines, normalized to HEL-2, an ANIL cell line with low CD93 expression that does not induce cytokine production (linear regression analysis).

FIG. 2O is a set of graphs showing GFP expression in mock-transduced, CD93-28z, or CD93-BBz CAR T cells co-cultured with AML cells stably expressing GFP at a 1:1 E:T ratio, measured in an Incucyte™ for 72 h; p<0.0001 for mock vs CD93 CAR for each cell line (2 way ANOVA, summary data from experiments from n=2 donors for Kasumi-1 and n=3 donors for OCI-AML3 and THP-1). The top trace in each panel represents the experiment with mock-transduced T cells, while the lower two traces in each panel represent experiments with CD93-28z (the bottom trace) or CD93-BBz CAR T cells, respectively.

FIGS. 3A-3N are sets of graphs showing an in vivo leukemic activity of CD93-CAR T cells in murine xenograft models. FIG. 3A is a carton showing a schematic of the THP-1 xenograft model. NSG mice were sublethally irradiated (200 cGy) on day −7 and then injected via tail vein on day −6 with THP-1 stably expressing GFP/luciferase. BLI was performed on day 0 to quantify engraftment in order to randomize the treatment groups (n=5 per group), then mice were injected via tail vein with mock-transduced, CD93-28z, or CD93-BBz CAR T cells. BLI was measured weekly to quantify AML burden. Peripheral blood was collected at day 14 to measure CAR T cell expansion. FIG. 3B is a line chart showing average BLI for THP-1-engrafted mice with leukemic progression in those treated with mock-transduced CAR T cells (black, the top trace), or CD93-28z (blue, the bottom trace) or CD93-BBz (purple, the middle trace) CAR T cells. p<0.0001 at weeks 2 and 3 post-CAR treatment (2 way ANOVA, data include experiments with n=2 donors). FIG. 3C is a set of fluorescence photographs showing that reduction of leukemic burden (THP-1) was observed in xenografted mice treated with CD93 CAR T cells but not in those treated with mock-transduced mice (n=5 in each group), as measured by BLI radiance and displayed colorimetrically. Of note, mice experienced GVHD around 4 weeks post-CAR treatment prior to natural death from leukemic progression, so survival analysis without confounding was not possible. FIG. 3D is a set of graphs showing BLI of individual mice treated with mock vs CD93-28z CAR or CD93-BBz CAR. FIG. 3E is a bar chart showing measurement of CD4+(left-side bars) and CD8+(right-side bars) T cell expansion at 2 weeks post-CAR injection. CD4+ and CD8+ total T cells/μl were measured in the peripheral blood by flow cytometry after RBC lysis (unpaired t test). FIG. 3F is a carton showing a schematic of a patient-derived xenograft model. NSG mice were sublethally irradiated (200 cGy) 1 day prior to injection via tail vein of SU555 primary patient-derived AML. Leukemic engraftment was evaluated by BMA every 3-4 weeks until 20 weeks after engraftment when AML percentage within the BM was at least 10% on average among the mice in each group randomized to receive either mock-transduced (n=17), CD93-28z (n=15), or CD93-BBz (n=20) CAR T cells. CAR T cells were injected via tail vein, then BMA was done 1 week and q3-4 weeks thereafter, and leukemic clearance and T cell expansion and persistence were monitored. FIG. 3G is a graph showing CD93 expression of PDX sample SU555 (blue, the right-side trace) compared to isotype (red, the left-side trace). FIG. 3H is a bar chart showing AML clearance, monitored by flow cytometry of BMA at 1 and every 3-5 weeks after CAR T cell injection (unpaired t test). The left-side bars represent experiments with mock-transduced T cells, the middle bars represent experiments with CD93 28z CAR T cells, and the right-side bars represent experiments with CD93 BBz CAR T cells. FIG. 3I is a line graph showing survival of mice until 100 days after injection (log-rank Mantel Cox test). FIG. 3J is a graph showing T cell persistence measured from BMA as described in FIG. 3H. Data in FIGS. 311-3J include experiments from n=3 donors. FIG. 3K is a set of flow cytometry graphs showing that CD93 CAR T cells induce massive cell death within BM 1 week after CAR T cell infusion. BMA from mice treated with mock-transduced T cells confirms AML engraftment by gating on live cells (FSC vs SSC), non-mouse RBCs (mTer119), human hematopoietic cells (mCD45 vs hCD45), and AML (CD33 vs CD3). FSC and SSC gating shows very few live cells in CD93 CAR T cell treated groups, precluding further analysis of leukemic clearance at this time point. FIG. 3L is a set of bar charts showing complete blood counts (CBCs) performed after CAR T treatment to determine bone marrow recovery, since BMA cannot be performed weekly. CBC was analyzed from peripheral blood 2 weeks post-CAR infusion. The mock-treated mice had a significantly lower hematocrit and trend toward thrombocytopenia and leukocytosis compared to CD93 CAR T cell-treated mice, consistent with leukemic progression in the mock group and leukemic control in the CAR-treated groups (unpaired t test). The bars in each panel represent, from left to right, experiments with mock-transduced T cells, CD93 28z CAR T cells, and CD93 BBz CAR T cells, respectively. FIG. 3M is a set of flow cytometry graphs showing representative dot plots in a mouse with patient-derived, SU555 AML, 4 weeks after treatment with mock-transduced CAR T cells. Live cells were gated on human CD45 then further analyzed for CD33 and CD3 expression to segregate human AML cells (CD33+CD3−) and human T cells (CD33−CD3+). CD33 positive cells were also analyzed for CD93 expression in the panel on the far right. FIG. 3N is a set of flow cytometry graphs showing representative dot plots for bone marrow aspirate collected from mice treated with CD93-28z or CD93-BBz, using T cells analyzed similarly as in FIG. 3M. For each treatment group, representative dot plots are shown for a mouse without detectable leukemia in the bone marrow and a mouse with relapsed AML.

FIGS. 4A-4F are sets of graphs showing that CD93 CAR T cells do not affect hematopoietic progenitor viability or regenerative capacity. FIG. 4A is a set of bar charts showing cytokine production levels by, from left to right, mock-transduced, CD93-28z, or CD93-BBz CAR T cells, respectively, when incubated alone, with THP-1 cells as a positive control, or with 2 separate cord blood samples after CD34+ selection (“CB #1” and “CB #2”). Supernatant was collected at 18 h and cytokine production (IFNγ and IL-2) was measured by ELISA (2 way ANOVA, data representative of experiments using n=2 donors). FIG. 4B is a bar chart comparing numbers of CFU-G/M/GM (the top block in each bar) and CFU/E (the bottom shaded block in each bar) colonies in CAR-treated samples against CD34+ cells alone. Cells were sorted on CD34 after co-culturing with mock-transduced, CD93-28z, or CD93-BBz CAR T cells, then suspended in methylcellulose and incubated for 14 d, prior to quantification and comparison. Graph represents normalization to mock averaged over 2 cord blood samples.

FIGS. 4C and 4D are sets of bar charts comparing proportions (quantified by flow cytometry) of the CD34+ cell subset in mock or CD93 CAR transduced T cells, after an 18 h co-culturing of the CAR T cells and cord-blood derived CD34+ cells, either separated by CD38+(black, the lower bar block)/CD38− (grey, the upper bar block) (FIG. 4C) or proportions of CMPs (green, the top bar block), GMPs (light blue, the second bar block from top), HSCs (dark blue, the third bar block from top), or all other cells (the bottom bar block) (FIG. 4D). The gating strategy is detailed in FIGS. 3K-3L. FIG. 4E is a set of graphs showing back-gating strategy prior to phenotypic analysis of hematopoietic progenitor subsets. CD34+ cells isolated by positive selection from cord blood underwent flow cytometry, and were first gated on live cells (PI−), non-debris (FSC vs SSC), singlets (SSC vs SSH and FSC vs FSH), and non-committed lineage negative cells (Lin−). FIG. 4F is a set of graphs showing gating strategy of lineage negative, hematopoietic progenitor populations. After 18 h co-culturing with CAR T cells, CD34+CD38+ cells were gated on CD10− cells, which were then gated on CD45RA and CD123 to analyze CMP (CD45RA−CD123+), GMP (CD45+CD123+), and MEP (CD45RA−CD123−). CD34+CD38− cells were separated into HSC (CD45RA−CD90+), MPP (CD45RA−CD90−), and LMPP (CD45RA+CD90−).

FIGS. 5A-5N are sets of graphs showing expression of CD93 and CD123 on normal myeloid and endothelial cells. FIG. 5A is a graph showing quantification of CD93 expression on normal tissue by immunohistochemistry. CD93 antibody was applied to a normal tissue microarray and stained samples were digitized with the Leica SCN400 scanner. H-scores were calculated on a scale of 0-300 based on the formula: % positive cells×staining intensity (staining intensity determined on a 0-3 descriptive scale, 0=no staining, 1=low, 2=clearly positive, 3=strong expression). FIG. 5B is a set of graphs showing immunohistochemistry using CD93 antibody on a tissue microarray with a panel of normal tissues (magnification equivalent to 20×, insets highlight endothelial cell staining). UMAP projection of single cells from a lung tissue, colored either by patient sample (FIG. 5C) or leiden gene expression cluster (FIG. 5D). FIG. 5E is a set of graphs showing UMAP projection of single cells, colored by expression of hematopoietic marker genes: PTPRC (CD45) is expressed primarily within clusters 0, 2, 4, 6, 9, 12, and 15; CD14 is expressed primarily within clusters 0, 1, 5, 6, and 8; CD3D is expressed in cluster 2. FIG. 5F is a set of graphs showing UMAP projection of single cells, colored by expression of endothelial marker genes marking different endothelial subsets: CLDN5 is expressed in all endothelial clusters (1, 5, and 13); VWF in stalk-like endothelial cells (cluster 1); EDNRB is expressed in a cluster that contains tip-like endothelial cells (cluster 5); CCL21 is expressed only in a cluster that represents endothelial venules (cluster 13). FIG. 5G is a set of graphs showing UMAP projections of single cells, colored by expression of CD93 and IL3RA (CD123) demonstrated expression in endothelial sub-clusters (most highly expressed in cluster 1). FIG. 5H is a violin plots displaying the expression level of tissue-specific markers representative of each cluster. A similar analysis was performed on scRNA-seq data from non-malignant pancreatic tissue. UMAP projection of single cells, colored either by patient sample (N=11) (FIG. 5I) or leiden gene expression cluster (FIG. 5J). FIG. 5K is a set of graphs showing UMAP projection of single cells, colored by expression of endothelial marker genes marking different endothelial subsets, in which CLDN5 and VWF mark an endothelial population (cluster 1). FIG. 5L is a set of graphs showing UMAP projection of single cells, colored by expression of hematopoietic marker genes, in which PTPRC (CD45) and CD14 are expressed primarily within cluster 7. FIG. 5M is a set of graphs showing UMAP projections of single cell, colored by expression of CD93 and IL3RA (CD123) demonstrated expression in endothelial cluster 1. CD33 and CD38 were not expressed at baseline in endothelial cells. FIG. 5N is a violin plots displaying the expression level of tissue-specific markers representative of each cluster, with particular focus on endothelial and hematopoietic cells.

FIGS. 6A-6V are sets of graphs illustrating that the on-target, off-tumor toxicity of CD93 CAR T cells to endothelial cells can be mitigated with an inhibitory CAR-based NOT-gate strategy. FIG. 6A is a set of graphs showing flow cytometry data on CD93 expression on endothelial cell lines (iHUVEC and TIME) and AML cell line (THP-1), compared to FMO negative control. FIG. 6B is a set of bar charts comparing levels of cytokine (e.g., IFNγ: the upper panel; IL-2: the bottom panel) production and secretion by CD93-28z CAR T cells (the right-side bar in each experiment), compared to mock-transduced T cells (the left-side bar in each experiment), when exposed to endothelial cell lines (iHUVEC and TIME) at a 1:1 E:T ratio. THP-1 AML cell line was included as a positive control (2 way ANOVA, summary data include experiments from n=4 donors). FIG. 6C is set of bar charts for experiments similar to FIG. 6B, comparing the cytokine levels of mock-transduced (the left-side bar in each E:T ratio group), CD93-28z CAR-transduced (the middle bar in each E:T ratio group), or CD93-BBz CAR-transduced (the right-side bar in each E:T ratio group) T cells under different E:T ratio groups. FIG. 6D is a set of bar charts showing that human CD93 CAR T cells do not cross-react with murine CD93. CD93-28z and CD93-BBz CAR T cells produce IFNγ and TL-2 when stimulated with plate-bound human CD93-Fc protein but not when stimulated with plate-bound murine CD93-Fc. Cytokine production of CD93-CARs after exposure to decreasing concentration of plate-bound human or murine CD93-Fc (indicated by black triangles, ranging from 5 μg/ml to 0.05 μg/ml; “−”: without any CD93-Fc) is compared to mock-transduced T cells. FIG. 6E is a set of graphs showing expression of CD93 and other AML targets in murine endothelial cells. UMAP projection of single cells from dissociation of lung (the left-side panels) or pancreas (the right-side panels) from the Tabula Muris database is shown. Multi-colored plots highlight cell ontogeny based on gene expression profiles. The same clusters are then overlaid with expression profiles of endothelial cell marker CLDN5, or AML targets CD93, CD123, and CD38. Analyses were performed in murine lung and pancreas cells. FIG. 6F is a cartoon showing a schematic of a NOT-gated CAR T cell system. FIG. 6G is a graph showing that a truncated version of CD19 (extracellular and transmembrane domains) was stably expressed on iHUVEC endothelial cells to generate iHUVEC-19 cells that represent “on-target, off-tumor” cells as described in the instant application. Expression of CD93 and CD19 on iHUVEC-19 (orange), iHUVEC (grey), and THP-1 (red) cells is shown. FIG. 6H is a cartoon showing detailed schematic of a CD93-28z activating CAR (aCAR) and a CD19 inhibitory CAR (iCAR) engineered with a CD19-specific scFv, CD8a transmembrane domain, and an endodomain derived from inhibitory receptors with immunoreceptor tyrosine-based inhibitory motifs (ITIMs), including, e.g., PD-1 and TIGIT. A construct with no intracellular signaling domain (Pdel) serves as a control. FIG. 6I is a bar chart showing a summary of co-transduction efficiencies of NOT-gated CARs (e.g., “CD93-28z/Pdel” refers to a combination of an aCAR molecule of CD93-28z and an iCAR molecule of CD19-Pdel; “CD93-28z/PD-1” refers to a combination of an aCAR molecule of CD93-28z and an iCAR molecule of CD19-PD-1) compared to controls. Blocks in each bar represent, from the top to the bottom, T cells expressing both the aCAR and the iCAR (“Dbl pos”), T cells expressing only the aCAR (“CD93-aCAR+”), T cells expressing only the iCAR (“CD19-iCAR+”), and T cells expressing none of the aCAR and the iCAR (“Dbl neg”). FIG. 6J is a line chart showing expansion of NOT-gated CARs in the first 10 days after CD3/28 activation, as measured by total cell counts. FIG. 6K is a set of flow cytometry dot lots demonstrating co-transduction efficiency of mock, CD93-28z alone, and CD93-28z/iCARs. CD93 CAR was detected with CD93-Fc and CD19 CAR was detected with anti-CD19 idiotype. FIG. 6L is a set of bar charts showing CD4/8 T cell ratios in NOT-gated CAR T cells. Results from T cell products derived from 2 separate donors are shown. FIGS. 6M-6P are graphs similar to FIGS. 6I-6L, respectively, but showing results for more NOT-gate CAR systems. FIG. 6Q is a set of graphs comparing expression of exhaustion markers PD-1 and TIM3 in NOT-gated CAR T cells, compared to mock-transduced and CD93-28z CAR T cells as controls. FIG. 6R is a set of bar charts comparing ELISA results of supernatants collected after co-culturing of THP-1, iHUVEC, or iHUVEC-CD19 with, from left to right in each group of treatment, mock-transduced T cells, CD93-28z aCAR T cells, or NOT-gated CAR T cells (including iCAR molecules containing Pdel, PD-1, or TIGIT, respectively). Results are represented as fold change compared to Pdel control (2 way ANOVA, summary data include experiments from n=2 donors). FIG. 6S is a set of graphs showing that the existence of a non-cancer cell recognizable by the iCAR molecule in NOT-gated CAR T cells does not eliminate the cytotoxicity of the T cells. Mock-transduced, CD93-28z-transduced, or NOT-gated CARs (with iCARs containing PD-1 or Siglec 5)-transduced T cells were exposed to THP-1 cells expressing GFP/luciferase alone (the left panel), both the THP-1 cells and iHUVEC cells (the middle panel), or both the THP-1 cells and iHUVEC-CD19 cells (the right panel). The fluorescence after each treatment was counted through time for comparison in fold changes (they-axis). FIG. 6T is a set of line charts showing the results of similar experiments as in FIG. 6S, but using more NOT-gated CAR T systems. FIG. 6U is a set of bar charts comparing cytokine production levels of, from left to right, mock-transduced (“Mock”), CD93-28z-transduced (“CD93-28z”), or various NOT-gated CAR-transduced T cells (i.e., CD93-Pdel, CD93-PD-1, CD93-Sig 5, CD93-Sig 2, CD93-Sig 6, and CD93-TIGIT, respectively; for example, “CD93-Pdel” refers to a combination of an aCAR molecule of CD93-28z and an iCAR molecule of CD19-Pdel), at different E:T ratios. FIG. 6V is a set of line charts comparing proliferation rates of T cells expressing mock, aCAR alone (i.e., CD93-28z), or CD93 aCAR/CD19 iCAR CAR constructs, when labeled with Nuclight red and co-cultured with HUVEC or HUVEC-19 target cells. T cell proliferation was measured over 96 h by Incucyte assay.

FIGS. 7A-7I are sets of graphs showing an overlap of surface protein expression in endothelial and AML cells, which is influenced by cytokines. FIG. 7A is a set of graphs showing flow cytometry data for expression of CD93 and other common AML targets (CD123, CD38, and CD33) by TIME cells, either untreated or treated for 24 h (red) or 48 h (green) with IFNγ and TNFα (100 ng/ml). FIG. 7B is a set of graphs showing quantification of endothelial surface protein expression, measured by fold MFI over untreated control. TIME cells were incubated with IFNγ and TNFα at varying doses (+++=100 ng/ml, ++=10 ng/ml, +=1 ng/ml) at 24 h (red) and 48 h (green), compared to untreated and FMO as negative controls. Bars for each treatment at 24 h or 48 h represent, from left to right, the FMO control, the untreated control, treatment with IFNγ and TNFα at 100 ng/ml, 10 ng/ml, or 1 ng/ml, respectively. FIG. 7C is a set of bar charts comparing transcriptional levels for selected AML targets on endothelial cells (measured as Log₂ TPM+1) from bulk RNAseq analysis performed on 2 endothelial cell lines (iH UVEC and TIME, shown here as increased marker expressions in average between the two cell lines) that were either untreated or treated with IFNγ, TNFα, or IFNγ and TNFα at 10 ng/ml for 24 h. FIGS. 7D-7E are graphs showing Gene Set Enrichment Analysis of the TIME and endothelial cell line transcriptome in response to inflammatory cytokines. Resting cells were compared to cells stimulated with IFNγ and TNFα. Positive enrichment scores indicate the gene set is enriched in the cytokine treated condition. Analysis was performed using the KEGG subset of the Canonical Pathway Collection (FIG. 7D) or the Transcription Factor Targets Collection (FIG. 7E). The top 15 results are shown. FIG. 7F is a table showing PSCAN analysis to identity overrepresented transcription factor binding motifs in the promoter regions of CD123 and CD38. Selected results are shown. FIG. 7G is a graph showing that RNA was isolated from 2 endothelial cell lines (iHUVEC and TIME) and 3 AML cell lines (THP-1, NOMO-1, and Kasumi-1) that were untreated or treated with IFNγ and TNFα (10 ng/ml). FIG. 7H is a graph showing a heatmap of genes that meet criteria for differential expression in AML or endothelial cells reveals clusters of genes with or without exposure to IFNγ and TNFα. Z-scores highlight differences among the cell types and treatment groups. FIG. 7I is a flow chart showing proposed model for gene expression analysis for rational combinatorial targeting (with NOT- or AND-gated CARs) to overcome endothelial toxicity from an AML-directed CAR.

FIGS. 8A-8C are sets of graphs showing functions of NOT-gated CAR-T systems with different elements, such as iCARs having various hinge/TM or internal signaling domains or aCARs targeting various antigens. FIG. 8A is a cartoon representation showing structures of NOT-gated CAR-T systems containing CD19-specific iCARs having various ITIM-containing internal signaling domains, with either a CD28 hinge-transmembrane domain or an IgG4 hinge+ native transmembrane domain (i.e., the TM domain from the ITIM-containing molecule). FIG. 8B is a set of bar graphs comparing cytokine production levels of T cells transduced with, from left to right in each treatment group, mock, CD93-28z alone, or NOT-gated CAR systems having CD93-28z and iCAR molecules with various hinge/TM domains and/or internal signaling domains (i.e., CD19-specific iCARs containing Pdel and CD8 TM, Pdel and IgG4 hinge/native TM, PD-1 and CD8 TM, PD-1 and IgG4 hinge/native TM, Sig 6 and CD8 TM, Sig 6 and IgG4 hinge/native TM, Sig 10 and CD8 TM, or Sig 10 and IgG4 hinge/native TM), after being exposed to control (“CAR alone”), THP-1 cells, iHUVEC cells, or iHUVEC-CD19 cells at an E:T ratio of 1:1. FIG. 8C is a set of graphs similar to FIG. 8B, but the experiments were performed at an E:T ratio of 1:4.

FIGS. 9A-9D are sets of graphs showing functions of NOT-gated CAR-T systems with different elements, such as aCAR molecule recognizing other antigens. FIG. 9A is a cartoon representation showing the structure of an aCAR specifically binding to HER2, using an scFv recognizing HER2, and an iCAR construct as described herein. FIG. 9B is a flow cytometry chart comparing HER2+ cancer cells (“Tumor”) and HER2+CD19+ non-cancer cells (“Healthy”). FIG. 9C is a set of flow cytometry charts for T cells expressing only the aCAR molecule (the up-left panel) or both the aCAR and one of the various iCAR molecules. FIG. 9D is a bar chart containing IL-2 production levels of T cells expressing, from left to right in each group, aCAR only or aCAR+ iCAR (containing, e.g., Pdel, PD-1, or Siglec 6), upon exposure to anti-CD19 idiotype antibodies.

### DETAILED DESCRIPTION OF THE DISCLOSURE

The present disclosure relates generally to, inter alia, recombinant cells expressing an inhibitory chimeric antigen receptor (iCAR) polypeptide that can be used, alone or in combination with an activating CAR (aCAR) polypeptide for logic gatings (e.g., NOT-gates), to reduce the capacity of the recombinant cells (e.g., T cells) to inhibit growth or kill a non-cancer cell, while maintaining or increasing the capacity of the recombinant cells to inhibit growth or kill a cancer cell expressing a target antigen recognizable by the aCAR molecule. In some embodiments, the non-cancer cell expresses both the target antigen recognizable by the aCAR molecule and a target antigen recognizable by the iCAR molecule. Without the iCAR molecule, the non-cancer cell may be recognized by the aCAR and, as a result, inhibited or killed by the activated recombinant cell, adding to the “on-target, off-tumor” side effect of the aCAR-only T therapy. In some embodiments, the iCAR molecule has an immunoreceptor tyrosine-based inhibitory motif (ITIM), which, when activated, inhibits activation of the aCAR molecule and the resulting activation of the recombinant cells expressing both the iCAR and the aCAR. The disclosure also provides compositions and methods useful for making such recombinant cells or CAR polypeptides, as well as methods for the detection and treatment of conditions, such as diseases (e.g., cancer).

CAR T cells are cytotoxic lymphocytes engineered to express a receptor that combines a tumor antigen recognition domain, normally in the form of an scFv, with endodomains that impart effector functions derived from the native T cell receptor and costimulatory proteins. Anti-tumor efficacy is often accompanied by hematopoietic toxicity due to shared antigen expression within the hematopoietic system (known as “on-target, off-tumor” toxicity/side effect) (Gill et al., Preclinical targeting of human acute myeloid leukemia and myeloablation using chimeric antigen receptor-modified T cells. Blood. 2014; 123(15):2343-2354; Kenderian et al., CD33-specific chimeric antigen receptor T cells exhibit potent preclinical activity against human acute myeloid leukemia. Leukemia. 2015; 29(8):1637-1647; Drent et al., Pre-clinical evaluation of CD38 chimeric antigen receptor engineered T cells for the treatment of multiple myeloma. Haematologica. 2016; 101(5):616-625; Laborda et al., Development of A Chimeric Antigen Receptor Targeting C-Type Lectin-Like Molecule-1 for Human Acute Myeloid Leukemia. Int J Mol Sci. 2017; 18(11); Tashiro et al., Treatment of Acute Myeloid Leukemia with T Cells Expressing Chimeric Antigen Receptors Directed to C-type Lectin-like Molecule 1. Mol Ther. 2017; 25(9):2202-2213; Jetani et al., CAR T-cells targeting FLT3 have potent activity against FLT3(−)ITD(+) AML and act synergistically with the FLT3-inhibitor crenolanib. Leukemia. 2018; 32(5):1168-1179). However, many have reasoned that hematopoietic toxicity is acceptable if AML CAR T cell therapy is administered in the context of myeloablation as a bridge to hematopoietic cell transplant (HCT) (Tasian et al., Optimized depletion of chimeric antigen receptor T cells in murine xenograft models of human acute myeloid leukemia. Blood. 2017; 129(17):2395-2407; Kim et al., Genetic Inactivation of CD33 in Hematopoietic Stem Cells to Enable CAR T Cell Immunotherapy for Acute Myeloid Leukemia. Cell. 2018; 173(6):1439-1453 e1419; Myburgh et al., Anti-human CD117 CAR T-cells efficiently eliminate healthy and malignant CD117-expressing hematopoietic cells. Leukemia. 202), or in conjunction with engineering strategies for CAR depletion or regulation (Kenderian et al., CD33-specific chimeric antigen receptor T cells exhibit potent preclinical activity against human acute myeloid leukemia. Leukemia. 2015; 29(8):1637-1647; Tasian et al., Optimized depletion of chimeric antigen receptor T cells in murine xenograft models of human acute myeloid leukemia. Blood. 2017; 129(17):2395-2407; Drent et al., Feasibility of controlling CD38-CAR T cell activity with a Tet-on inducible CAR design. PLoS One. 2018; 13(5):e0197349). Preclinical studies in xenograft models have shown encouraging anti-AML activity of CAR T cells directed against a number of different cell surface molecules (Gill et al., Preclinical targeting of human acute myeloid leukemia and myeloablation using chimeric antigen receptor-modified T cells. Blood. 2014; 123(15):2343-2354; Kenderian et al., CD33-specific chimeric antigen receptor T cells exhibit potent preclinical activity against human acute myeloid leukemia. Leukemia. 2015; 29(8):1637-1647; Laborda et al., Development of A Chimeric Antigen Receptor Targeting C-Type Lectin-Like Molecule-1 for Human Acute Myeloid Leukemia. Int J Mol Sci. 2017; 18(11); Tashiro et al., Treatment of Acute Myeloid Leukemia with T Cells Expressing Chimeric Antigen Receptors Directed to C-type Lectin-like Molecule 1. Mol Ther. 2017; 25(9):2202-2213; Jetani et al., CAR T-cells targeting FLT3 have potent activity against FLT3(−)ITD(+) AML and act synergistically with the FLT3-inhibitor crenolanib. Leukemia. 2018; 32(5):1168-1179; Myburgh et al., Anti-human CD117 CAR T-cells efficiently eliminate healthy and malignant CD117-expressing hematopoietic cells. Leukemia. 2020; Casucci et al., CD44v6-targeted T cells mediate potent antitumor effects against acute myeloid leukemia and multiple myeloma. Blood. 2013; 122(20):3461-3472; Chen et al., Targeting FLT3 by chimeric antigen receptor T cells for the treatment of acute myeloid leukemia. Leukemia. 2017; 31(8):1830-1834; Gomes-Silva et al., CD7 CAR T Cells for the Therapy of Acute Myeloid Leukemia. Mol Ther. 2019; 27(1):272-280; John et al., A Novel Anti-LILRB4 CAR-T Cell for the Treatment of Monocytic AML. Mol Ther. 2018; 26(10):2487-2495; Lynn et al., Targeting of folate receptor beta on acute myeloid leukemia blasts with chimeric antigen receptor-expressing T cells. Blood. 2015; 125(22):3466-3476; Mardiros et al., T cells expressing CD123-specific chimeric antigen receptors exhibit specific cytolytic effector functions and antitumor effects against human acute myeloid leukemia. Blood. 2013; 122(18):3138-3148; Tettamanti et al., Targeting of acute myeloid leukaemia by cytokine-induced killer cells redirected with a novel CD123-specific chimeric antigen receptor. Br J Haematol. 2013; 161(3):389-401; Wang et al., CAR-T cells targeting CLL-1 as an approach to treat acute myeloid leukemia. J Hematol Oncol. 2018; 11(1):7; Wang et al., Targeting FLT3 in acute myeloid leukemia using ligand-based chimeric antigen receptor-engineered T cells. J Hematol Oncol. 2018; 11(1):60; Westwood et al. Adoptive transfer of T cells modified with a humanized chimeric receptor gene inhibits growth of Lewis-Y-expressing tumors in mice. Proc Natl Acad Sci USA. 2005; 102(52):19051-19056; Yoshida et al., All-trans retinoic acid enhances cytotoxic effect of T cells with an anti-CD38 chimeric antigen receptor in acute myeloid leukemia. Clin Transl Immunology. 2016; 5(12):e116; Zhang et al., B7-H3-Targeted CAR-T Cells Exhibit Potent Antitumor Effects on Hematologic and Solid Tumors. Mol Ther Oncolytics. 2020; 17:180-189). However, only a few have been translated into early phase clinical trials, most notably CD123 and CD33 (Epperly et al., Harnessing T Cells to Target Pediatric Acute Myeloid Leukemia: CARs, BiTEs, and Beyond. Children (Basel). 2020; 7(2)). The few available published reports of the early clinical experience have shown muted effectiveness in patients compared to the preclinical data (Ritchie et al., Persistence and efficacy of second generation CAR T cell against the LeY antigen in acute myeloid leukemia. Mol Ther. 2013; 21(11):2122-2129; Wang et al., Treatment of CD33-directed chimeric antigen receptor-modified T cells in one patient with relapsed and refractory acute myeloid leukemia. Mol Ther. 2015; 23(1):184-191; Budde et al., Remissions of Acute Myeloid Leukemia and Blastic Plasmacytoid Dendritic Cell Neoplasm Following Treatment with CD123-Specific CAR T Cells: A First-in-Human Clinical Trial. American Society for Hematology 59th Annual Congress. 2017; 130(811); Cummins et al., Treating Relapsed/Refractory (RR) AML with Biodegradable AntiCD123 CAR Modified T Cells. American Society for Hematology 59th Annual Congress. 2017; 130:1359). Moreover, serious adverse events have been reported. The first two patients on a CD123 CAR trial developed grade 4 capillary leak syndrome, resulting in one patient death and termination of the study (Cellectis Reports Clinical Hold of UCART123 Studies [press release] at World Wide Web site at www.cellectis.com/en/press/cellectis-reports-clinical-hold-of-ucart123-studies2017). Expression of CD123 on endothelial cells may render them susceptible to on-target, off-tumor toxicity (Sun et al., IFN-gamma and TNF-alpha aggravate endothelial damage caused by CD123-targeted CAR T cell. Onco Targets Ther. 2019; 12:4907-4925), a factor that could have contributed to these clinical outcomes. These reports underscore the importance of extending preclinical studies of normal tissue toxicity for new myeloid targets beyond the hematopoietic system.

A system that controls CAR T cells similar to Boolean logic gates (e.g. NOT-gates), which modules T cell activity according to different recognizable cell surface antigens on cancer cells vs. non-cancer cells, would drastically increase safety, revolutionizing the field and greatly benefiting patients with cancer.

Within a typical activating chimeric antigen receptor (aCAR), the intracellular signaling domain (a.k.a., cytosolic signaling domain) generally refers to a cytoplasmic domain that transmits an activation signal to the cell expressing the CAR molecule, following binding of the extracellular domain of the aCAR to the corresponding ligand or antigen expressed by a target cell (e.g., a cancer cell). In some cases, the intracellular signaling domain includes a functional signaling domain derived from a stimulatory molecule. Traditional intracellular signaling domains almost always include CD3zeta, the prototypical “master switch” that elicits T cell activity (Irving and Weiss Cell 1991; 64:891-901; Letourneur and Klausner Science 1992; 255:79-82), as well as various optional costimulatory domains to enhance potency and persistence.

As described in greater detail below, the iCAR molecules have been designed and tested for their activities, alone or in combination with a traditional aCAR molecule to form NOT-gates, to reduce T cell activation (which results in, e.g., cytokine production and secretion, cytotoxicity, etc.) against a non-cancer cell expressing both a target antigen recognizable by the aCAR and a target antigen recognizable by the iCAR, while maintaining or increasing the T cell capacity to inhibit growth or kill a cancer cell expressing only the target antigen for the aCAR but not the target antigen for the iCAR. In some embodiments, the intracellular signaling domain of these iCAR molecules has an immunoreceptor tyrosine-based inhibitory motif (ITIM), which, when activated, inhibits activation of the aCAR molecules.

Compositions of the iCAR polypeptides, alone or in combination with the aCAR polypeptides, as well as nucleic acid molecules encoding these polypeptides are also provided. The disclosure also provides compositions and methods useful for producing such recombinant cells having the iCAR polypeptides, as well as methods for the prevention and/or treatment of conditions, such as cancer.

All publications and patent applications mentioned in this disclosure are herein incorporated by reference to the same extent as if each individual publication or patent application was specifically and individually indicated to be incorporated by reference.

### General Experimental Procedures

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference. As appropriate, procedures involving the use of commercially available kits and reagents are generally carried out in accordance with manufacturer defined protocols and/or parameters unless otherwise noted.

### Definition

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, a “host cell” refers to a cell for introduction of a nucleic acid and/or a polypeptide (e.g., the CAR molecules) described herein and/or a cell for expressing a nucleic acid or a polypeptide described herein. Host cells can be either untransformed cells or cells that have already been introduced with at least one nucleic acid molecule (e.g., the CAR molecules) described herein. A “recombinant cell” refers to a cell having genetic modifications and/or having introduced nucleic acids and/or polypeptides described herein.

As used herein, a “subject” or an “individual” includes animals, such as human and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates (e.g., mammals and non-mammals), such as non-human primates, sheep, dogs, cats, cows, chickens, rodents, mice, amphibians, reptiles, etc.

The term “vector” is used herein to refer to a nucleic acid molecule or sequence capable of transferring or transporting another nucleic acid molecule. For example, a vector can be used as a gene delivery vehicle to transfer a gene into a cell. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i), (ii), etc., are presented merely for ease of reading the specification and claims. The use of headings in the specification or claims does not require the steps or elements be performed in alphabetical or numerical order or the order in which they are presented.

As can be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As can also be understood by one skilled in the art all language such as “up to”, “at least”, “greater than”, “less than”, and the like include the number recited and refer to ranges which can be subsequently broken down into sub-ranges as discussed above. Finally, as can be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions of the Disclosure

As described in greater detail below, the invention provides, inter alia, compositions of recombinant cells expressing iCARs, alone or in combination with aCARs to form logic gatings (e.g., NOT-gates), and combinations of CARs that can be used for logic gating to target and eliminate specific cells (e.g., cancer cells) but not other cells (e.g., non-cancer cells). In some embodiments, the iCARs have an immunoreceptor tyrosine-based inhibitory motif (ITIM) in their intracellular signaling domains, capable of inhibiting activation of aCARs having an immune receptor tyrosine based activation motif (ITAM) in their intracellular signaling domains. Also provided are combinations of these iCARs, alone or in combination with aCARs to form logic gatings (e.g., NOT-gates), for their inhibitory functions. Also provided are recombinant nucleic acids encoding such iCARs or iCAR+aCAR combinations (NOT-gated systems).

In some embodiments, the invention provides a composition of a recombinant cell containing the iCAR polypeptide and/or the nucleic acid encoding such iCAR polypeptide described herein. Such recombinant cell may be capable of expressing such iCAR polypeptide. In some embodiments, the invention provides a composition of a recombinant cell containing a “NOT-gating” system, containing, e.g., at least one of the iCAR polypeptides and at least one of the aCAR polypeptides, described herein, and/or the nucleic acids encoding such system. Such recombinant cell may be capable of expressing both the CAR polypeptides.

### Chimeric Antigen Receptors (Cars)

As described above, the iCAR polypeptides of the present disclosure include (i) an extracellular ligand-binding domain (a.k.a., extracellular antigen-binding domain, or ECD) having a binding affinity for a ligand (or an antigen); (ii) a transmembrane domain (TMD); and (iii) an intracellular signaling domain (a.k.a., cytosolic signaling domain, or ICD), when binding of the ligand/antigen to the extracellular ligand-binding domain activates the intracellular signaling domain. In some embodiments, the iCAR polypeptides described herein have an immunoreceptor tyrosine-based inhibitory motif (ITIM) in their intracellular signaling domains. When activated, the iCAR polypeptides may inhibit activate of an activating CAR (aCAR) polypeptide, which contains (i) an extracellular ligand-binding domain (a.k.a., extracellular antigen-binding domain, or ECD) having a binding affinity for a ligand (or an antigen); (ii) a transmembrane domain (TMD); and (iii) an intracellular signaling domain (a.k.a., cytosolic signaling domain, or ICD), when binding of the ligand/antigen to the extracellular ligand-binding domain activates the intracellular signaling domain. In some embodiments, the aCAR polypeptides described herein have an immune receptor tyrosine based activation motif (ITAM) in their intracellular signaling domains. In some embodiments, a cancer cell (or other target cells related to a disease or condition) expresses only the target antigen recognizable by the aCAR polypeptide, but not the target antigen recognizable by the iCAR polypeptide. In some embodiments, a non-cancer cell (e.g., a normal or healthy cell) expresses both the target antigen recognizable by the aCAR polypeptide and the target antigen recognizable by the iCAR polypeptide. In some embodiments, a cancer cell (or other target cells related to a disease or condition) expresses only the target antigen recognizable by the aCAR polypeptide but not the target antigen recognizable by the iCAR polypeptide, and a non-cancer cell (e.g., a normal or healthy cell) expresses both the target antigen recognizable by the aCAR polypeptide and the target antigen recognizable by the iCAR polypeptide, thereby the cancer cell may be recognized by the aCAR and, as a result, inhibited or killed by a recombinant cell (e.g., T cells) expressing both the aCAR and the iCAR, when the non-cancer cell may be recognized by both the aCAR and the iCAR and, due to the inhibition by the iCAR, not inhibited or killed by the recombinant cell. In some embodiments, the term “expressing a target antigen” (e.g., the target antigen recognizable by the aCAR polypeptide and/or the target antigen recognizable by the iCAR polypeptide) means expressing such target antigen in an amount and/or a ratio (e.g., the ratio between the amount of the expressed target antigen recognizable by the aCAR polypeptide to the amount of the expressed target antigen recognizable by the iCAR polypeptide) equal to or higher than a threshold amount and/or ratio, thereby rendering the aCAR and/or the iCAR polypeptide being capable of being activated by binding to the specific target antigen, thus promoting or reducing the activation of the recombinant cell expressing such CAR polypeptide(s). In some embodiments, the term “not expressing a target antigen” (e.g., the target antigen recognizable by the aCAR polypeptide and/or the target antigen recognizable by the iCAR polypeptide) means expressing such target antigen in an amount and/or a ratio (e.g., the ratio between the amount of the expressed target antigen recognizable by the iCAR polypeptide to the amount of the expressed target antigen recognizable by the aCAR polypeptide) lower than a threshold amount and/or ratio, thereby preventing the aCAR and/or the iCAR polypeptide(s) from being activated by binding to the specific target antigen, or preventing the activation of the recombinant cell expressing such CAR polypeptide(s) through the activation of the aCAR and/or the iCAR polypeptide(s). Such threshold amount and/or ratio may be determined by a skilled artisan from experimentation using positive and/or negative CAR polypeptides.

In some embodiments, the disclosed CARs (e.g., either aCARs or iCARs) have the above listed domains in (i)-(iii) in a N-terminal to C-terminal direction. In some embodiments, the disclosed CARs (e.g., either aCARs or iCARs) are described in a format of X-Y-Z, wherein X represents the ligand/antigen recognizable by the extracellular ligand-binding domain, Y represents the transmembrane domain, and Z represents the intracellular signaling domain. For example, “CD93-CD28TM-CD3ζ” refers to a CAR molecule having an extracellular ligand-binding domain which specifically binds to CD93, a CD28 hinge/transmembrane domain, and a CD3ζ intracellular signaling domain. Combinations of CAR molecules follow the same format. For example, “CD93-CD28TM-CD3ζ+CD19-CD8TM-PD-1” refers to a CAR combination (a NOT gate) having two CAR molecules, including one aCAR as described above, and another iCAR having an extracellular ligand-binding domain which specifically binds to CD19, a CD8 hinge/transmembrane domain, and a PD-1 intracellular signaling domain. Exemplary aCAR molecules may have a structure of CD93-CD28TM-CD3ζ, CD93-CD28TM-CD28-CD3ζ, HER2-TM-CD3ζ, or HER2-TM-CD28-CD3ζ, when the intracellular signaling domain may be substituted with signaling domains derived from other molecules. Exemplary iCAR molecules may have a structure of CD19-8TM-ITIM, CD19-IgG4hinge-TM-ITIM, when the ITIM-containing intracellular signaling domain may contain any signaling domains derived from PD-1, CTLA-4, LAIR1 (LIR-1), TIGIT, Sig 2 (CD22), Sig 5, Sig 6, Sig 10, BTLA, LAG3, CD300a, or SIRPα. Possible ITIM-containing intracellular signaling domain may also contain or be derived from, e.g., other Sig (sialic acid-binding immunoglobulin-type lectins, or Siglec) family members, such as Sig 1 (Sialoadhesin), Sig 3 (CD33), Sig 4 (Mag), Sig 7, Sig 8, Sig 9, Sig 11, Sig 12, Sig 13, Sig 14, Sig 15, Sig 16, Sig 17, etc.

The idea of using inhibitory CAR (iCAR) molecules to inhibit T-cell-mediated immune responses was previously reported, such as in Fedorov et al., Sci Trans Med 2013; 5(215): 215ra172; Hwang et al. PNAS 2021; 118(12): e2022410118; Hamburger et al. Mol Immunol 2020; 128: 298-310, as well as in WO 2015/142314, WO 2016/075612, and WO 2020/065406, all incorporated herein by references to their entirety. In the instant invention, an iCAR molecule is designed to, upon recognizing a target antigen expressed by a non-cancer cell, antagonize and reduce activation of an aCAR molecule, and the resulting T cell activation and cytotoxicity against the non-cancer cell expressing both antigens for the aCAR and the iCAR, while maintaining or increasing the capacity of the T cell for activation and cytotoxicity against a cancer cell expressing only a target antigen recognizable by the aCAR but not (or not sufficiently) the target antigen for iCAR. Thus an aCAR+ iCAR combination is named as a NOT-gated system, which is capable of mitigating the “on-target, off-tumor” side effect of traditional CAR T therapies, without dramatically losing cytokine production and cytotoxicity capacity of the CAR T cell against target cancers. In some embodiments, the iCAR molecule described herein, when activated by binding to its target antigen, is capable of interacting with the aCAR molecule described herein and reducing or inhibiting activation of the aCAR molecule, when the aCAR molecule specifically binds to the target antigen for the aCAR molecule. For example, most Siglecs (Sigs), such as CD22 (Sig 2) and the CD33 (Sig 3)-related family, contain ITIMs in their cytosolic region, which act to down-regulate signaling pathways involving phosphorylation, such as those induced by ITAMs in the cytosolic region of the aCAR molecules described herein. In some embodiments, the tyrosine contained within the ITIM is phosphorylated after ligand binding and acts as a docking site for SH2 domain-containing proteins like SHP phosphatases. This leads to de-phosphorylation of cellular proteins, down-regulating activating signaling pathways. In some embodiments, the iCAR molecules described herein may directly reduce or inhibit activation of the aCAR molecule described herein. For example, when activated, the iCAR molecules described herein may recruit a SH2-containing phosphatase to inhibit phosphorylation of the ITAM region of the aCAR and to prevent or reduce activation of the aCAR molecule. In some embodiments, the iCAR molecules described herein may directly reduce or inhibit activation of the recombinant cell expressing the iCAR and the aCAR molecules described herein. For example, when activated, the iCAR molecules described herein may prevent or reduce activation of an endogenous or exogenous protein crucial for activating the recombinant cell, e.g., by recruiting a SH2-containing phosphatase to inhibit phosphorylation of the crucial endogenous or exogenous protein, and to prevent or reduce activation of the recombinant cell. In some embodiments, the iCAR molecule described herein prevents or inhibits activation of the recombinant cell, even the aCAR molecule described herein is activated by specifically binding to the target antigen. In some embodiments, the iCAR molecule is introduced together with the aCAR molecule (as a NOT-gated system) into the recombinant cell. In some embodiments, the iCAR molecule is introduced alone into the recombinant cell. Under this scenario, the iCAR molecule may modulate the activation of the recombinant cell alone or in combination with an aCAR molecule or an aCAR-like molecule (e.g., an endogenous receptor) pre-existing in the recombinant cell (e.g., either exogenously or endogenously).

In some embodiments, the disclosed CARs further include one or more hinge domains and/or costimulatory domains. Description of these corresponding CAR constructs follows the same format as above, adding the optional hinge and/or costimulatory domain in the correspond place (in N-terminal to C-terminal direction). For example, “CD93-CD28TM-CD28-CD3ζ” refers to an aCAR molecule having an extracellular ligand-binding domain which specifically binds to CD93, a CD28 hinge/transmembrane domain, a CD28 costimulatory domain, and a CD3ζ intracellular signaling domain.

### Extracellular Ligand (Antigen)-Binding Domains (ECD)

In some embodiments, the ECD of the CAR polypeptides (e.g., both the aCARs and the iCARs) disclosed herein has a binding affinity for one or more target ligands (or antigens, which are used interchangeably in the instant application). In some embodiments, the target ligand is expressed on a cell surface, or is otherwise anchored, immobilized, or restrained on a cell surface. Non-limiting examples of suitable ligand types include cell surface receptors, adhesion proteins, carbohydrates, lipids, glycolipids, lipoproteins, and lipopolysaccharides that are surface-bound, integrins, mucins, and lectins. In some embodiments, the ligand is a protein. In some embodiments, the ligand is a carbohydrate. In some embodiments, the ligand for the aCAR is expressed by a target cell (e.g., a cancer/tumor cell). In some embodiments, the ligand for the aCAR is an adaptor molecule specifically recognizing a target cell (e.g., a cancer/tumor cell). In some embodiments, the ligand for the aCAR is a biomarker for a specific disease, disorder, or condition (e.g., a cancer/tumor). In some embodiments, the ligand for the aCAR is expressed both by a target cell (e.g., a cancer/tumor cell) and another cell (e.g., a non-cancer/tumor, normal, or healthy cell). In some embodiments, the ligand for the iCAR is expressed by a target cell (e.g., a non-cancer/tumor, normal, or healthy cell). In some embodiments, the ligand for the iCAR is an adaptor molecule specifically recognizing a target cell (e.g., a non-cancer/tumor, normal, or healthy cell). In some embodiments, the ligand for the iCAR is expressed only by a target cell (e.g., a non-cancer/tumor, normal, or healthy cell) but not another cell (e.g., a cancer/tumor cell). Non-limiting examples of suitable ligand include CD93, CD123, CD38, CD33, HER2, or CD44v6, as well as those described in the below section titled “antigens”. Some exemplary antigens are expressed on endothelial cells, such as CD123, LeY, CD33, TCRs (e.g., TCR recognizing PR1 or WT-1), CD7, CD38, FRO, CD44v6, CLL1, FLT3, etc.

In some embodiments, the ECD of the CAR polypeptides (e.g., both the aCARs and the iCARs) disclosed herein includes an antigen-binding moiety that binds to one or more target antigens. In some embodiments, the antigen-binding moiety includes one or more antigen-binding determinants of an antibody or a functional antigen-binding fragment thereof, including, at least, a ligand-binding domain of an antibody, an antigen-binding fragment, an antibody mimetic, a receptor, or a ligand for a targeted receptor. One skilled in the art upon reading the present disclosure can readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a V_{H} domain, a V_{L} domain, an Fv fragment, a VNAR domain, and a V_{HH} domain, or a functional fragment thereof. In some embodiments, the antigen-binding moiety includes a heavy chain variable region and a light chain variable region. In some embodiments, the antigen-binding moiety includes a scFv. In some embodiments, the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof. In some embodiments, the receptor is NKG2D or a biologically active fragment thereof. In some embodiments, the ligand for a targeted receptor is an IL-13 polypeptide, an IL-13 mutein, cholorotoxin, or a biologically active fragment thereof.

The antigen-binding moiety can include naturally-occurring amino acid sequences or can be engineered, designed, or modified so as to provide desired and/or improved properties, e.g., binding affinity. Generally, the binding affinity of an antibody or an antigen-binding moiety for a target antigen (e.g., CD93, CD123, CD19, HER2, etc.) can be calculated by the Scatchard method described by Frankel et al., Mol. Immunol, 16: 101-106, 1979. In some embodiments, binding affinity can be measured by an antigen/antibody dissociation rate. In some embodiments, a high binding affinity can be measured by a competition radioimmunoassay. In some embodiments, binding affinity can be measured by ELISA. In some embodiments, antibody affinity can be measured by flow cytometry. An antibody that “selectively binds” a target antigen (e.g., CD93, CD123, CD19, HER2, etc.) is an antibody that binds the target antigen with high affinity and does not significantly bind other unrelated antigens but binds the antigen with high affinity, e.g., with an equilibrium constant (K_{D}) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a CAR polypeptide of the present disclosure. For example, a CAR polypeptide with an ECD including an antibody specific for a CD93 antigen can target cells to CD93-expressing cancer cells. In some embodiments, the ECD of the CAR polypeptides disclosed herein is capable of binding a tumor-associated antigen (TAA) or a tumor-specific antigen (TSA). A skilled artisan can understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

In some embodiments, the ECD of the CAR polypeptides described herein (either aCARs or iCARs) contains an antibody or antigen-binding fragment thereof, which specifically binds to the target antigen. In some embodiments, the ECD of the CAR polypeptides described herein (either aCARs or iCARs) contains an scFv specifically binds to the target antigen. In some embodiments, the scFv contains a heavy chain variable domain and a light chain variable domain, optionally conjugated by a linker in either direction (e.g., H-L or L-H). There is no particular limitation on the linkers that can be used in the chimeric polypeptides described herein. In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The linker can also be a linker peptide sequence, as those described in Chen et al., Fusion Protein Linkers: Property, Design and Functionality. Adv Drug Deliv Rev. 2013; 65(10): 1357-1369 and those known in the art. For example, the linker may have stretches of Gly and Ser residues (“GS” linkers). An example of the most widely used flexible linker has the sequence of (Gly-Gly-Gly-Gly-Ser)ₙ or (G₄S)n. By adjusting the copy number “n”, the length of this GS linker can be optimized to achieve appropriate separation of the functional domains, or to maintain necessary inter-domain interactions. In principle, there are no particular limitations to the length and/or amino acid composition of the linker peptide sequence. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the heavy chain variable domain and the light chain variable domain of the scFv in the ECD of the CAR polypeptides described herein are conjugated by a GS linker, such as a (G₄S)₃ linker (SEQ ID NO: 11).

In some embodiments, the ECD of the aCAR polypeptide described herein has at least one of CDR H1, CDR H2, and CDR H3 heavy chain CDR domains having an amino acid sequence having at least 50%, at least 60%, at least 70%, at least 75%, at least 80%, at least 85%, at least 90%, at least 95%, at least 99% or 100% sequence identity to any one of SEQ ID NOs: 3-5. In some embodiments, the ECD of the aCAR polypeptide described herein has at least one of CDR L1, CDR L2, and CDR L3 light chain CDR domains having an amino acid sequence having at least 50%, at least 60%, at least 70%, at least 75%, at least 80%, at least 85%, at least 90%, at least 95%, at least 99% or 100% sequence identity to any one of SEQ ID NOs: 8-10. In some embodiments, the ECD of the aCAR polypeptide has a heavy chain variable domain having an amino acid sequence having at least 50%, at least 60%, at least 70%, at least 75%, at least 80%, at least 85%, at least 90%, at least 95%, at least 99%, or 100% sequence identity to SEQ ID NO: 1 or 2. In some embodiments, the ECD of the aCAR polypeptide has a light chain variable domain having an amino acid sequence having at least 50%, at least 60%, at least 70%, at least 75%, at least 80%, at least 85%, at least 90%, at least 95%, at least 99%, or 100% sequence identity to SEQ ID NO: 6 or 7. In some embodiments, the ECD of the aCAR polypeptide contains an amino acid sequence having at least 50%, at least 60%, at least 70%, at least 75%, at least 80%, at least 85%, at least 90%, at least 95%, at least 99%, or 100% sequence identity to any one of SEQ ID NOs: 12-15.

### Antigens

In the instant application, the terms “ligand(s)” and “antigen(s)” are used interchangeably to mean the target molecule(s) specifically recognized by the extracellular antigen-binding domain of the CAR molecule or CAR combinations (e.g., either iCARs, aCARs, or iCAR+aCAR combinations) described herein. In principle, there are no particular limitations with regard to suitable target antigens. In some embodiments of the disclosure, the antigen-binding moiety of the ECD is specific for an epitope present in an antigen that is expressed by a target cell. For example, the antigen for an aCAR may be a tumor-associated antigen (which may optionally also be expressed by a non-tumor cell), when the antigen for an iCAR may be an antigen expressed by only a non-tumor cell (e.g., a normal, healthy cell) but not a tumor cell. The tumor-associated antigen can be an antigen associated with a cancer or a tumor, e.g., a leukemia, such as acute myeloid leukemia (AML), myelodysplastic syndrome, myeloproliferative neoplasms, or acute myeloid leukemia with MLL rearrangements. In some embodiments, the antigen-binding moiety is specific for an epitope present in a tissue-specific antigen. In some embodiments, the antigen-binding moiety is specific for an epitope present in a disease-associated antigen. Tumors often refers to a subgroup of cancers when an uncontrolled growth of cells occurs in solid tissue such as an organ, muscle, or bone. In the instant application, the terms “tumors” and “cancers” are generally used interchangeably to mean cells having an uncontrolled growth, unless specified otherwise.

In some embodiments, the antigen is selected from the group consisting of CD93, CD123, CD38, CD33, HER2, CD19, CD44v6, and a human leukocyte antigen (HLA). In some embodiments, the aCAR polypeptides disclosed herein include an ECD including an antigen-binding moiety that binds CD93. In some embodiments, the CAR polypeptides disclosed herein include an ECD including an antigen-binding moiety having at least one of CDR H1, CDR H2, CDR H3, CDR L1, CDR L2, and CDR L3 domains having an amino acid sequence exhibiting at least 50%, at least 60%, at least 70%, at least 75% at least 80%, at least 85%, at least 90%, at least 95%, at least 96%, at least 97%, at least 98%, at least 99%, or 100% sequence identity to any one of SEQ ID NOs: 3-5 and 8-10. In some embodiments, the antigen-binding moiety has a variable heavy chain and/or a variable light chain domain having an amino acid sequence exhibiting at least 50%, at least 60%, at least 70%, at least 75% at least 80%, at least 85%, at least 90%, at least 95%, at least 96%, at least 97%, at least 98%, at least 99%, or 100% sequence identity to any one of SEQ ID NOs: 1, 2, 6, and 7. The percent identity as used herein refers to two or more sequences or subsequences that are the same or have a specified percentage of nucleotides or amino acids that are the same over a specified region. The two or more sequences or subsequences may be compared and aligned for maximum correspondence over a comparison window or designated region, as measured by, e.g., a BLAST or BLAST 2.0 sequence comparison algorithms, or by manual alignment and visual inspection. See e.g., the NCBI web site at ncbi.nlm.nih.gov/BLAST. This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. The amino acid substitution(s) may be a conservative amino acid substitution, for example at a non-essential amino acid residue in the CDR sequence(s). A “conservative amino acid substitution” is understood to be one in which the original amino acid residue is substituted with an amino acid residue having a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

One exemplary target antigen for the aCAR molecules described herein is CD93. CD93 is a cell surface lectin that is highly expressed at diagnosis and relapse in a sizable fraction of AML cases (Coustan-Smith et al. Universal monitoring of minimal residual disease in acute myeloid leukemia. JCI Insight. 2018; 3(9)). In addition, CD93 has been implicated in leukemogenesis and maintenance of a cycling, non-quiescent population of LSCs in MLL-rearranged (MLLr) AML (Iwasaki et al., CD93 Marks a Non-Quiescent Human Leukemia Stem Cell Population and Is Required for Development of MLL-Rearranged Acute Myeloid Leukemia. Cell Stem Cell. 2015; 17(4):412-421). The instant application teaches an exemplary second-generation aCAR incorporating a novel CD93-specific scFv, with demonstrated leukemic clearance in preclinical xenograft models and mitigated hematopoietic toxicity. CD93 is largely absent from non-hematopoietic tissues. However, immunohistochemistry and single cell RNA-sequencing (scRNA-seq) revealed expression in endothelial cells, with further proofs that the CD93-specific aCAR targeted endothelial cell lines. In the same scRNA-seq datasets, CD123 expression mirrored CD93 on endothelial cell subsets, and endothelial cells exposed to inflammatory cytokines expressed both CD123 and CD38, indicating that a risk for endothelial cell toxicity may be significant for AML targets beyond CD93. The disclosed iCARs and NOT-gated systems (e.g., having iCARs and aCARs) were demonstrated to mitigate endothelial cell toxicity that is caused by shared antigen expression between AML and endothelial cells. In addition, bulk RNA-seq was carried out to more broadly characterize the AML and endothelial surfaceome in order to provide an inventory of antigens with suitable expression profiles for single or combinatorial targeted AML immunotherapies, using the iCARs or the NOT-gated systems.

CD93 has been alternatively named as complement component 1 Q subcomponent receptor 1 [C1qR(P), or C1QR1], C1q Receptor 1, CDw93, DJ737E23.1, C1qRP, ECSM3, or matrix-remodeling-associated protein 4 (MXRA4), C1q/MBL/SPA receptor, etc. Human CD93 has 652 amino acids and a molecular weight of 68560 Da. The complete amino acid sequence for human CD93 may be found under GeneBank Reference number NP_036204.2 and Uniprot database entry number Q9NPY3.

Target antigens for the aCAR and the iCAR in a NOT-gated system are different. The target antigen for the aCAR molecule is usually a cancer or tumor-associated antigen, which is expressed in a sufficient amount by the cancer cell to be inhibited or killed. In some embodiments, the target antigen is not expressed by the non-cancer cell to be protected from inhibiting or killing by the activated recombinant cell (e.g., a T cell) expressing the NOT-gated system (e.g., aCAR and iCAR polypeptides). In some embodiments, the target antigen is also expressed by the non-cancer cell. In some embodiments, the target antigen is expressed by the non-cancer cell in an insufficient amount to activate the recombinant cell through binding of the target antigen to the aCAR molecule. For example, it is generally believed that the threshold amount required for aCAR-induced cytolytic activity is more than about 2000 antigen molecules per cell (Majzner et al, Cancer Discovery 2020; 10(5):702-723; Fry et al, Nat Med. 2018; 24(1):20-28; Watanabe et al, Journal of Immunol 2015). In some embodiments, the non-cancer cell expresses a less amount of the target antigen for the aCAR molecule than the cancer cell, thereby resulting in a competitive advantage for the cancer cell against the non-cancer cell to bind to, and thus activate, the aCAR molecule. In some embodiments, the non-cancer cell expresses a comparable amount, or even more, of the target antigen for the aCAR molecule than the cancer cell. In some embodiments, the ratio of the amount of the expressed target antigens for the aCAR molecule on the cancer cell vs. the amount on the non-cancer cell is about less than 1:100, about 1:100, about 1:50, about 1:25, about 1:10, about 1:5, about 1:1, about 5:1, about 10:1, about 25:1, about 50:1, about 100:1, more than 100:1, more than 500:1, more than 1000:1, or even more.

The target antigen for the iCAR molecule is usually an antigen for normal or healthy non-cancer cells. In some embodiments, such antigen is a specific marker for endothelial cells. By binding to the target antigen, the iCAR may be activated to prevent or reduce T cell activation and cytotoxicity to the non-cancer cells. In some embodiments, the target antigen for the iCAR molecule is not expressed by the cancer cells. In some embodiments, the target antigen is expressed by the cancer cells in an insufficient amount to activate the iCAR molecule, thus maintaining the activation and cytotoxicity capacity of T cells against the cancer cells. In some embodiments, the cancer cells express a less amount of the target antigen for the iCAR than the non-cancer cells, thereby resulting in a competitive advantage for the non-cancer cells against the cancer cells to bind to, and thus activate, the iCAR molecule. In some embodiments, the ratio of the amount of the expressed target antigens for the iCAR molecule on the non-cancer cell vs. the amount on the cancer cell is about 5:1, about 10:1, about 25:1, about 50:1, about 100:1, more than 100:1, more than 500:1, more than 1000:1, or even more.

In some embodiments, any antigen for the aCAR or iCAR molecules is expressed by the cancer cell or the non-cancer cell in an amount of less than 100, about 100, about 500, about 1000, about 2000, about 5000, about 10000, about 20000, about 50000, about 100000, or more.

### Hinge Domains

As described above, the CAR polypeptides described herein (either aCARs or iCARs) may have an optional hinge domain. Within a chimeric antigen receptor, the term “hinge domain” generally refers to a flexible polypeptide connector region disposed between the targeting moiety (ECD) and the TMD. These sequences are generally derived from IgG subclasses (such as IgG1 and IgG4), IgD and CD8 (e.g., CD8alpha) domains, of which IgG1 has been most extensively used. “Derived from” refers to the origin or source of a molecule, and may include naturally occurring, recombinant, unpurified, or purified molecules. Nucleic acid or polypeptide molecules are considered “derived from” when they include portions or elements assembled in such a way that they produce a functional unit. The portions or elements can be assembled from multiple sources provided that they retain evolutionarily conserved function. In some embodiments, the derivative nucleic acid or polypeptide molecules include substantially the same sequence as the source nucleic acid or polypeptide molecule. For example, the derivative nucleic acid or polypeptide molecules of the present disclosure may have at least 80%, 85%, 90%, 95%, 98%, 99%, or 100% sequence identity to the source nucleic acid or polypeptide molecule. The term “recombinant” nucleic acid molecule, polypeptide, and/or cell, as used in the instant application, refers to a nucleic acid molecule, polypeptide, and/or cell that has been altered through human intervention. As non-limiting examples, a recombinant nucleic acid molecule can be one which: 1) has been synthesized or modified in vitro, for example, using chemical or enzymatic techniques, or recombination of nucleic acid molecules; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. A non-limiting example of a recombinant protein is a chimeric antigen receptor as provided herein.

In some embodiments, the hinge domain provides structural flexibility to flanking polypeptide regions. The hinge domain may consist of natural or synthetic polypeptides. In recent years, several studies of the hinge domain mainly focused on the following aspects: (1) reducing binding affinity to the Fcγ receptor, thereby eliminating certain types of off-target activation; (2) enhancing the single-chain variable fragment (scFv) flexibility, thereby relieving the spatial constraints between particular epitopes targeted on tumor antigens and the CAR's antigen-targeting moiety; (3) reducing the distance between an scFv and the target epitope(s); and (4) facilitating the detection of CAR expression using anti-Fc reagents. Nevertheless, the influences of the hinge domain on CAR T cell physiology are not well understood.

It can be appreciated by those skilled in the art that hinge domains may improve the function of the CAR polypeptides described herein by promoting optimal positioning of the antigen-binding moiety in relationship to the portion of the antigen recognized by the same. It can be appreciated that, in some embodiments, the hinge domain may not be required for optimal CAR activity. In some embodiments, a beneficial hinge domain having a short sequence of amino acids promotes CAR activity by facilitating antigen binding by, e.g., relieving any steric constraints that may otherwise alter antibody binding kinetics. The sequence encoding the hinge domain may be positioned between the antigen recognition moiety and the TMD. In some embodiments, the hinge domain is operably linked downstream of the antigen-binding moiety and upstream of the TMD. In case a CAR polypeptide described herein has an optional hinge domain, the format to describe the hinge domain and the TMD domain may be “H/TM” or “H-TM”. For example, CD19-CD28H/TM-PD-1 or CD19-CD28H-TM-PD-1 represents the same CAR molecule having a CD28 hinge domain and a CD28 transmembrane domain (TMD or TM), when IgG4H/CD4TM represents a CAR molecule having a hinge domain derived from IgG4 or including an IgG4 hinge domain, plus a CD4 transmembrane domain or a transmembrane domain derived from the CD4 transmembrane domain.

The hinge sequence can generally be any moiety or sequence derived or obtained from any suitable molecule. For example, in some embodiments, the hinge sequence can be derived from the human CD8 molecule or a CD28 molecule and any other receptors that provide a similar function in providing flexibility to flanking regions. The hinge domain can have a length of from about 4 amino acid (aa) to about 50 aa, e.g., from about 4 aa to about 10 aa, from about 10 aa to about 15 aa, from about aa to about 20 aa, from about 20 aa to about 25 aa, from about 25 aa to about 30 aa, from about 30 aa to about 40 aa, or from about 40 aa to about 50 aa. Suitable hinge domains can be readily selected and can be of any of a number of suitable lengths, such as from 1 amino acid (e.g., Gly) to 20 aa, from 2 aa to 15 aa, from 3 aa to 12 aa, including 4 aa to 10 aa, 5 aa to 9 aa, 6 aa to 8 aa, or 7 aa to 8 aa, and can be 1, 2, 3, 4, 5, 6, or 7 aa. Non-limiting examples of suitable hinge domains include a CD8 hinge domain, a CD28 hinge domain, a CD4 hinge domain, a PD-1 hinge domain, a CD2 hinge domain, a CTLA4 hinge domain, or an IgG4 hinge domain. In some embodiments, the hinge domain can include regions derived from a human CD8a (a.k.a. CD8a) molecule or a CD28 molecule and any other receptors that provide a similar function in providing flexibility to flanking regions. In some embodiments, the CAR disclosed herein includes a hinge domain derived from a CD8 hinge domain. In some embodiments, the hinge domain can include one or more copies of the CD8 hinge domain. In some embodiments, the CAR disclosed herein includes a hinge domain derived from a CD28 hinge domain. In some embodiments, the hinge domain can include one or more copies of the CD28 hinge domain. In some embodiments, the CAR disclosed herein includes a hinge domain derived from a CD4 hinge domain. In some embodiments, the hinge domain includes one or more copies of the CD4 hinge domain. In some embodiments, the CAR disclosed herein includes a hinge domain derived from an IgG4 hinge domain. In some embodiments, the hinge domain can include one or more copies of the IgG4 hinge domain.

### Costimulatory Domains

As described above, the CAR polypeptides described herein (either aCARs or iCARs) may have an optional costimulatory domain. Generally, the costimulatory domain suitable for the CAR polypeptides disclosed herein can be any one of the costimulatory domains known in the art. Examples of suitable costimulatory domains that can enhance cytokine production and include, but are not limited to, costimulatory polypeptide sequences derived from 4-1BB (CD137), CD27, CD28, OX40 (CD134), and costimulatory inducible T-cell co-stimulator (ICOS) polypeptide sequences. Accordingly, in some embodiments, the costimulatory domain of the CARs disclosed herein is selected from the group consisting of a costimulatory 4-1BB (CD137) polypeptide sequence, a costimulatory CD27 polypeptide sequence, a costimulatory CD28 polypeptide sequence, a costimulatory OX40 (CD134) polypeptide sequence, and a costimulatory inducible T-cell co-stimulator (ICOS) polypeptide sequence. In some embodiments, the CARs disclosed herein include a costimulatory domain derived from a costimulatory 4-1BB (CD137) polypeptide sequence. In some embodiments, the CARs disclosed herein include a costimulatory 4-1BB (CD137) polypeptide sequence. In some embodiments, the CARs disclosed herein include a costimulatory domain derived from a costimulatory CD28 polypeptide sequence. In some embodiments, the CARs disclosed herein include a costimulatory CD28 polypeptide sequence.

### Transmembrane Domains (TMD)

Generally, the transmembrane domain (also referred to as transmembrane region) suitable for the CAR polypeptides disclosed herein (either aCARs or iCARs) can be any one of the TMDs known in the art. Without being bound to theory, it is believed that the TMD traverses the cell membrane, anchors the CAR to the cell surface, and connects the ECD to the ICD, thus impacting expression of the CAR on the cell surface. Examples of suitable TMDs include, but are not limited to, a CD28 TMD, a CD8 TMD, a CD4 TMD, a CD3 TMD, a CTLA-4 TMD, an OX40 TMD, a 4-1BB TMD, a CD2 TMD, and a PD-1 TMD. Accordingly, in some embodiments, the TMD is derived from a CD28 TMD, a CD8 TMD, a CD4 TMD, a CD3 TMD, a CTLA4 TMD, an OX40 TMD, a 4-1BB TMD, a CD2 TMD, and a PD-1 TMD. In some embodiments, the TMD includes a CD28 TMD, a CD4 TMD, a CD8 TMD, a CD3 TMD, a CTLA4 TMD, an OX40 TMD, a 4-1BB TMD, a CD2 TMD, and a PD-1 TMD. In some embodiments, the CAR disclosed herein include a TMD derived from a CD8 (e.g., CD8alpha). In some embodiments, the CAR polypeptides disclosed herein include a CD8 (e.g., CD8alpha) TMD. In some embodiments, the CAR disclosed herein include a TMD derived from a CD28. In some embodiments, the CAR disclosed herein include a CD28 TMD. In some embodiments, the CAR disclosed herein include a TMD derived from a CD4. In some embodiments, the CAR disclosed herein include a CD4 TMD.

### Extracellular Spacer

In some embodiments, the CARs disclosed herein (either aCARs or iCARs) further include an optional extracellular spacer domain including one or more intervening amino acid residues that are positioned between the ECD and an optional hinge domain. In some embodiments, the extracellular spacer domain is operably linked downstream to the ECD and upstream to an optional hinge domain. In principle, there are no particular limitations to the length and/or amino acid composition of the extracellular spacer. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as an extracellular spacer. In some embodiments, the extracellular spacer includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the extracellular spacer includes about 1 to 10, about 5 to 15, about 10 to 20, about 15 to 25, about 20 to 40, about 30 to 50, about 40 to 60, about 50 to 70 amino acid residues. In some embodiments, the extracellular spacer includes about 40 to 70, about 50 to 80, about 60 to 80, about 70 to 90, or about 80 to 100 amino acid residues. In some embodiments, the extracellular spacer includes about 1 to 10, about 5 to 15, about 10 to 20, about 15 to 25 amino acid residues. In some embodiments, the length and amino acid composition of the extracellular spacer can be optimized to vary the orientation and/or proximity of the ECD and an optional hinge domain to one another to achieve a desired activity of the CARs. In some embodiments, the orientation and/or proximity of the ECD and an optional hinge domain to one another can be varied and/or optimized as a “tuning” tool or effect that would enhance or reduce the efficacy of the CARs. In some embodiments, the orientation and/or proximity of the ECD and an optional hinge domain to one another can be varied and/or optimized to create fully functional or partially functional versions of the CARs. In some embodiments, the extracellular spacer domain includes an amino acid sequence corresponding to an IgG4 hinge domain and an IgG4 CH2-CH3 domain.

### Intracellular Signaling Domain

In some embodiments, the CARs disclosed herein (either aCARs or iCARs) include an intracellular signaling domain or internal signaling region capable of being activated upon binding of the ECD domain to the target antigen.

In some embodiments, the intracellular signaling domains of the aCAR molecules descried herein have an immune receptor tyrosine based activation motif (ITAM), e.g., CD3ζ. In some embodiments, the intracellular signaling domains of the aCAR molecules descried herein do not have an immune receptor tyrosine based activation motif (ITAM). By “CD3ζ polypeptide” is meant a protein having at least 75, 80, 85, 90, 95, 96, 97, 98, 99 or 100% identity to NCBI Reference No: NP_932170 or a fragment thereof that has activating or stimulatory activity.

In some embodiments, the intracellular signaling domains of the iCAR molecules descried herein have an immunoreceptor tyrosine-based inhibitory motif (ITIM). In some embodiments, the activated iCAR may inhibit activation of the aCAR and thus the T cells expressing the aCAR and the iCAR molecules. ITIM is a conserved sequence of amino acids that is found intracellularly in the cytoplasmic domains of many inhibitory receptors of the non-catalytic tyrosine-phosphorylated receptor family found on immune cells (Dushek et al., Non-catalytic tyrosine-phosphorylated receptors. Immunological Reviews. 2012; 250 (1): 258-276; Veillette et al., Negative regulation of immunoreceptor signaling. Annual Review of Immunology 2002; 20 (1): 669-707). These immune cells may include T cells, B cells, NK cells, dendritic cells, macrophages and mast cells. ITIMs have similar structures of S/I/V/LxYxxI/V/L, where x is any amino acid, Y is a tyrosine residue that can be phosphorylated, S is the amino acid Serine, I is the amino acid Isoleucine, and V is the amino acid Valine (Coxon et al., ITIM receptors: more than just inhibitors of platelet activation. Blood. 2017; 129 (26):3407-3418). ITIMs recruit SH2 domain-containing phosphatases, which inhibit cellular activation. ITIM-containing receptors often serve to target ITAM-containing receptors, resulting in an innate inhibition mechanism within cells. Id. ITIM bearing receptors have important role in regulation of immune system allowing negative regulation at different levels of immune responses. A list of more than 135 ITIM-containing proteins was reported (Staub et al., Systematic identification of immunoreceptor tyrosine-based inhibitory motifs in the human proteome. Cellular Signalling. 2004; 16 (4): 435-56), and further expanded by studying rare human SNPs. Exemplary ITIM-containing domains for the iCAR molecules described herein include intracellular signaling domain of or derived from PD-1, CTLA-4, LAIR1 (LIR-1), TIGIT, Sig 2, Sig 5, Sig 6, Sig 10, BTLA, LAG3, CD300a, or SIRPα. Possible ITIM-containing domains for the iCAR molecules may also include any one of the intracellular signaling domains of or derived from other Sig or Siglec proteins, such as Sig 1 (Sialoadhesin), Sig 3 (CD33), Sig 4 (Mag), Sig 7, Sig 8, Sig 9, Sig 11, Sig 12, Sig 13, Sig 14, Sig 15, Sig 16, Sig 17, etc. For a summary of the Siglec family members, see the review of Crocker et al., Siglecs and their roles in the immune system. Nature Reviews Immunology 2007; 7:255-266.

One skilled in the art can appreciate that the complete amino acid sequence of a CAR polypeptide of the disclosure can be used to construct a back-translated gene. For example, a DNA oligomer containing a nucleotide sequence coding for a given CAR can be synthesized. For example, several small oligonucleotides coding for portions of the desired CAR or antibody can be synthesized and then ligated. The individual oligonucleotides typically contain 5′ or 3′ overhangs for complementary assembly.

In addition to generating desired CARs via expression of nucleic acid molecules that have been altered by recombinant molecular biological techniques, a subject CAR in accordance with the present disclosure can be chemically synthesized. Chemically synthesized polypeptides are routinely generated by those of skill in the art.

Once assembled (by synthesis, recombinant methodologies, site-directed mutagenesis or other suitable techniques), the DNA sequences encoding a CAR as disclosed herein can be inserted into an expression vector and operably linked to an expression control sequence appropriate for expression of the CAR in the desired transformed host. Proper assembly can be confirmed by nucleotide sequencing, restriction mapping, and expression of a biologically active polypeptide in a suitable host. As is known in the art, in order to obtain high expression levels of a transfected gene in a host, take should be taken to ensure that the gene is operably linked to transcriptional and translational expression control sequences that are functional in the chosen expression host.

### Combinations of Chimeric Antigen Receptors (Cars) (Logic “Not” Gates)

As described above, the CAR polypeptides of the present disclosure can be combined to form Boolean logic gates (e.g. NOT) to response to a combination of extracellular ligands/antigens. In one aspect, provide herein is a composition or a system having at least two CAR polypeptides of the CAR polypeptides described herein, including, e.g., an activating CAR (aCAR) and an inhibitory CAR (iCAR). The CAR combinations described herein, e.g., forming logic gatings, are capable of activating cells (e.g., T cells) expressing the CAR combinations to, e.g., inhibit growth and/or kill a target cancer cell (which expresses the target antigen recognizable by the aCAR) but not a target non-cancer cell (which expresses both the target antigen recognizable by the aCAR and the target antigen recognizable by the iCAR). In some embodiments, the aCAR molecule has an intracellular signaling domain with or without an ITAM. In some embodiments, the iCAR molecule has an intracellular signaling domain having an immunoreceptor tyrosine-based inhibitory motif (ITIM).

To form an “NOT” gate CAR molecule combination, the iCAR molecule recognizes its target antigen expressed by a non-cancer cell (e.g., on the cell membrane) and inhibits the activation of the aCAR and the T cell to mitigate the inhibition or killing of the non-cancer cell, while maintaining or increasing the capacity of the T cell against the cancer cell which does not express, or does not express a sufficient number of, the target antigen for the iCAR. The amount of the target antigen for the iCAR expressed by the non-cancer cells vs. the amount expressed by the cancer cells, or the ratio of these amounts, may determine the effectiveness of the protection of the non-cancer cells or the degree of missing of the cancer cells for inhibition or elimination.

In some embodiments, a NOT-gating system is formed by at least one of the iCAR molecules and at least one of the aCAR molecules, as described herein, which are both introduced into a cell to produce the recombinant cell expressed herein. In some embodiments, a NOT-gating system is formed by an iCAR molecule introduced into a cell and an aCAR molecule or an aCAR-like molecule (e.g., an endogenous or exogenous receptor) pre-existing in the cell (e.g., either endogenously or exogenously).

In some embodiments, CARs in the “NOT”-gated combination/system have a same TMD, optional hinge domain, and/or optional costimulatory domain. In some embodiments, CARs in the “NOT”-gated combination/system have different TMDs, optional hinge domains, and/or optional costimulatory domains. For example, different domains, as described herein, may be used for the CARs in the “NOT”-gated combination/system to reduce aggregation of the CARs, and/or to modulate the potency and/or the specificity (i.e., the “leakiness”) of the CAR combination.

### Mutations to Chimeric Antigen Receptors (Cars)

Multiple mutations can be introduced to the CAR polypeptides of the present disclosure, either used alone or in NOT-gated combinations/systems. Mutations include, at least, substitution, deletion, insertion, or other methods known in the art. The purpose of mutations may include, for example, to enhance the potency of the CAR polypeptide (or the CAR polypeptide combination), to enhance the stability (e.g., half-life) of the CAR polypeptide, to enhance the expression of the CAR polypeptide, to enhance the solubility and/or to reduce aggregation of the CAR polypeptide, to manipulate modifications of the CAR polypeptide during expression, to manipulate binding of the CAR polypeptide to its binding partner(s), to enhance the purification of the CAR polypeptide, to reduce ubiquitination and/or degradation of the CAR polypeptide, to reduces the background activation levels of the cell (i.e., the “leakiness”) when some but not all CAR polypeptides in a NOT-gated combination/system are activated, to reduces aggregation of the multiple CAR polypeptides in a NOT-gated combination/system in absence of the ligand, etc.

### Cells Expressing the Car Polypeptides

In some embodiments, the iCAR polypeptides (alone or in NOT-gated combinations) are expressed in a cell and are capable of inhibiting the activation of the cell by an aCAR polypeptide, upon binding to the corresponding ligand(s)/antigen(s). In principle, there are no particular limitations with regard to suitable cells for expressing and activation by the CAR polypeptides. In some embodiments, the cell is an immune cell. For example, the immune cell may include, at least, a T cell, a natural killer (NK) cell, a macrophage, a natural killer T (NKT) cell, or an iPSC-derived T cell. In some embodiments, the cell is a non-immune cell. The cells may be any type of natural or artificial cells and/or of any origins. By expressing the CAR polypeptides (alone or in NOT-gated combinations/systems) described herein in these cells, an exemplary manipulating mechanism is introduced so that the cells may be activated and such activation may be detected. Thus, any type of cells that people want to study the activation may be used for expressing the CAR polypeptide described herein.

### Nucleic Acid Molecules

In one aspect, provided herein are various nucleic acid molecules including nucleotide sequences encoding an iCAR polypeptide of the disclosure, including expression cassettes, and expression vectors containing these nucleic acid molecules operably linked to heterologous nucleic acid sequences such as, for example, regulator sequences which allow in vivo expression of the iCAR polypeptide in a host cell or ex-vivo cell-free expression system.

Nucleic acid molecules of the present disclosure can be nucleic acid molecules of any length, including nucleic acid molecules that are generally between about 0.5 Kb and about 50 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb. In some embodiments, the nucleic acid molecules of the disclosure are between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the recombinant nucleic acid includes a nucleic acid sequence encoding an iCAR that includes an extracellular ligand-binding domain having a binding affinity for a ligand; a transmembrane domain; and an intracellular signaling domain. In some embodiments, the iCAR encoded by the nucleic acid sequence further includes an optional hinge domain and/or costimulatory domain.

In some embodiments, a composition has at least two recombinant nucleic acids, each including a nucleic acid sequence encoding an iCAR polypeptide or an aCAR polypeptide described herein to form an NOT-gated CAR combination/system. In some embodiments, these at least two recombinant nucleic acids are conjugated together. In some embodiments, these at least two recombinant nucleic acids are in a single chain of a recombinant nucleic acid.

In some embodiments, the recombinant nucleic acid molecule described herein is operably linked to a heterologous nucleic acid sequence.

In some embodiments, the recombinant nucleic acid molecule is further defined as an expression cassette or a vector. It can be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the CAR polypeptide as disclosed herein, which is operably linked to expression control elements, such as a promoter, and optionally, any other sequences or a combination of other nucleic acid sequences that affect the transcription or translation of the coding sequence.

In some embodiments, the nucleotide sequence is incorporated into an expression vector. It can be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As can be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the vector is a vector derived from a lentivirus, an adeno virus, an adeno-associated virus, a baculovirus, or a retrovirus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 50%, 60%, 70%, 80%, 90%, 95%, 96%, 97, 98%, 99%, or 100% sequence identity to a CAR polypeptide disclosed herein (e.g., an iCAR).

The nucleic acid sequences encoding the CAR polypeptides can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

The nucleic acid molecules provided can contain naturally occurring sequences, or sequences that differ from those that occur naturally, but, due to the degeneracy of the genetic code, encode the same polypeptide, e.g., antibody for the ECD. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies for the ECD); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid molecules of the present disclosure can be introduced into a cell (i.e., a host cell), such as a human T cell, to produce a recombinant cell containing the nucleic acid molecule. Accordingly, some embodiments of the disclosure relate to methods for making a recombinant cell, including (a) providing a host cell capable of protein expression; and transducing the provided host cell with a recombinant nucleic acid of the disclosure to produce a recombinant cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be introduced into a host cell by viral or non-viral delivery vehicles known in the art to produce an engineered cell. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using zinc-finger proteins (ZNF), guide RNA directed CRISPR/Cas9, DNA-guided endonuclease genome editing NgAgo (Natronobacterium gregoryi Argonaute), or TALEN genome editing (transcription activator-like effector nucleases).

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculovirus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the CAR polypeptides of interest.

In some embodiments, the recombinant cell is a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a mouse cell. In some embodiments, the animal cell is a human cell. In some embodiments, the cell is a non-human primate cell. In some embodiments, the recombinant cell is an immune system cell, e.g., a B cell, a monocyte, a NK cell, a natural killer T (NKT) cell, a basophil, an eosinophil, a neutrophil, a dendritic cell, a macrophage, a regulatory T cell, a helper T cell (T_{H}), a cytotoxic T cell (TCTL), a memory T cell, a gamma delta (T6) T cell, another T cell, a stem cell (e.g., a hematopoietic stem cell), a stem cell progenitor (e.g., a hematopoietic stem cell progenitor) an induced pluripotent stem cell (iPSC)-derived NK cell, or an induced pluripotent stem cell (iPSC)-derived T cell.

In some embodiments, the immune system cell is a lymphocyte. In some embodiments, the lymphocyte is a T lymphocyte. In some embodiments, the lymphocyte is a T lymphocyte progenitor. In some embodiments, the T lymphocyte is a CD4+ T cell or a CD8+ T cell. In some embodiments, the T lymphocyte is a CD8+T cytotoxic lymphocyte cell. Non-limiting examples of CD8+T cytotoxic lymphocyte cell suitable for the compositions and methods disclosed herein include naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+T helper lymphocyte cell. Suitable CD4+T helper lymphocyte cells include, but are not limited to, naïve CD4+ T cells, central memory CD4+ T cells, effector memory CD4+ T cells, effector CD4+ T cells, CD4+ stem memory T cells, and bulk CD4+ T cells.

In some embodiments, the host cell described herein is a non-immune system cell. For example, the CAR molecules and/or the CAR molecule combinations described herein provide a method to modulate the activity of a cell expressing such CAR molecules and/or combinations, upon recognizing the corresponding extracellular ligand(s)/antigen(s). Generally, there are no particular limitations with regard to suitable host cell.

As outlined above, some embodiments of the disclosure relate to various methods for making a recombinant cell, including (a) providing a host cell capable of protein expression; and transducing the provided host cell with a recombinant nucleic acid of the disclosure to produce a recombinant cell. Non-limiting exemplary embodiments of the disclosed methods for making a recombinant cell can further include one or more of the following features. In some embodiments, the host cell is obtained by leukapheresis performed on a sample obtained from a subject, and the cell is transduced ex vivo. In some embodiments, the recombinant nucleic acid is encapsulated in a viral capsid or a lipid nanoparticle. In some embodiments, the methods further include isolating and/or purifying the produced cells. Accordingly, the recombinant cells produced by the methods disclosed herein are also within the scope of the disclosure.

Techniques for transforming a wide variety of the above-mentioned host cells and species are known in the art and described in the technical and scientific literature. For example, DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection. In some embodiments, the nucleic acid molecule is introduced into a host cell by a transduction procedure, electroporation procedure, or a biolistic procedure. Accordingly, cell cultures including at least one recombinant cell as disclosed herein are also within the scope of this application. Methods and systems suitable for generating and maintaining cell cultures are known in the art.

In a related aspect, some embodiments of the disclosure relate to cell cultures including at least one recombinant cell as disclosed herein, and a culture medium. Generally, the culture medium can be any one of suitable culture media for the cell cultures described herein. In some embodiments, the recombinant cell expresses a CAR described herein. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the CAR polypeptides, nucleic acids, recombinant cells, and/or cell cultures of the disclosure can be incorporated into compositions, including pharmaceutical compositions. Such compositions generally include the CARs, nucleic acids, recombinant cells, and/or cell cultures as described herein and a pharmaceutically acceptable carrier. Accordingly, in one aspect, some embodiments of the disclosure relate to pharmaceutical compositions for treating, preventing, ameliorating, reducing or delaying the onset of a health condition, for example a proliferative disease (e.g., cancer).

Accordingly, one aspect of the present disclosure relates to pharmaceutical compositions that include a pharmaceutically acceptable carrier and one or more of the following: (a) a CAR polypeptide of the disclosure; (b) a nucleic acid molecule of the disclosure; and/or (c) a recombinant cell of the disclosure. In some embodiments, the composition includes (a) a recombinant nucleic acid of the disclosure and (b) a pharmaceutically acceptable carrier. In some embodiments, the recombinant nucleic acid is encapsulated in a viral capsid or a lipid nanoparticle. In some embodiments, the composition includes (a) a recombinant cell of the disclosure and (b) a pharmaceutically acceptable carrier.

In certain embodiments, the pharmaceutical compositions in accordance with some embodiments disclosed herein include cell cultures that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art can appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

### Use of the Compositions

The compositions described herein, e.g., CARs (alone or in combinations), nucleic acids, recombinant cells, cell cultures, and/or pharmaceutical compositions, can be used for various conditions, as a system in response to extracellular signals (e.g., ligands/antigens).

For example, the compositions described herein may be used as a detection system for detecting certain ligands/antigens. As described herein, in principle, there are no particular limitations with regard to suitable target antigens and suitable host cells. In some embodiments, targeting a specific ligand/antigen or a specific profile of ligands/antigens, the corresponding CAR molecule(s) is prepared to having an extracellular antigen-binding domain (ECD) to specifically binding to the ligand(s)/antigen(s). The corresponding intracellular signaling domain(s) can then activate, after the ligand/antigen binding, the host cell to produce a signal to be detected, such as expressing a gene for detection (e.g., by detecting the expressed DNA/RNA/protein or any luminescence/fluorescence from the expressed protein). In some embodiments, such detection system may be used for detecting certain biomarkers in a biological sample, such as used for diagnosis of any disease or disorder.

The compositions described herein may also be used as a system to manipulate cell functions in response to certain ligands/antigens. In some embodiments, by sensing a specific ligand/antigen or a specific profile of ligands/antigens, expressed by a non-cancer cell, with the ECD(s) of the iCAR molecules in the NOT-gated systems, the activation and cytotoxicity of the host cell, induced by activation of the aCAR molecules in the NOT-gated systems upon binding to another ligand/antigen on the non-cancer cell, is inhibited. Such inhibition may mitigate the “on-target, off-tumor” side effect of CAR T therapies against non-cancer normal or healthy cells, but maintaining or increasing the capacity of CAR T therapies against the target cancer cells. Thus, a selective recognizing, inhibiting and/or killing of target cancer cells, but not target non-cancer cells may be achieved.

### Methods of Treatment

Administration of any one of the therapeutic compositions described herein, e.g., CARs, nucleic acids, recombinant cells, cell cultures, and/or pharmaceutical compositions, can be used in the diagnosis, prevention, and/or treatment of relevant conditions, such as proliferative diseases (e.g., cancer), with mitigated toxicity to non-cancer, normal or healthy cells. In some embodiments, the CARs, nucleic acids, recombinant cells, cell cultures, and/or pharmaceutical compositions as described herein can be incorporated into therapies and therapeutic agents for use in methods of preventing and/or treating an individual who has, who is suspected of having, or who may be at high risk for developing one or more health conditions, such as proliferative diseases (e.g., cancers). In some embodiments, the individual is a patient under the care of a physician.

Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a leukemia. In some embodiments, the cancer is acute myeloid leukemia (AML), myelodysplastic syndrome, myeloproliferative neoplasms, or acute myeloid leukemia with MLL rearrangements.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject with a mitigated “on-target, off-tumor” side effect.

Accordingly, in one aspect, some embodiments of the disclosure relate to methods for the prevention and/or treatment of a condition in a subject in need thereof, wherein the methods include administering to the subject a composition including one or more of: an iCAR polypeptide of the disclosure, a NOT-gated CAR combination/system, a recombinant nucleic acid of the disclosure, a recombinant cell of the disclosure, and/or a pharmaceutical composition of the disclosure.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more. In some embodiments, the methods include administering to the individual an effective number of the recombinant cells disclosed herein, wherein the recombinant cells inhibit the proliferation of the target cell and/or inhibit tumor growth of a target cancer in the subject compared to the proliferation of the target cell and/or tumor growth of the target cancer in subjects who have not been administered with the recombinant cells. In some embodiments, the administered composition inhibits the “on-target, off-tumor” side effect of the recombinant cell against non-cancer, normal, or healthy cells (e.g., endothelial cells). Inhibition includes a reduction of the measured inhibition or killing of the non-cancer, normal or healthy cells of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

Administration of the compositions described herein, e.g., CARs, nucleic acids, recombinant cells, cell cultures, and/or pharmaceutical compositions, can be used in the stimulation of an immune response. In some embodiments, CARs, nucleic acids, recombinant cells, cell cultures, and/or pharmaceutical compositions as described herein are administered to an individual after induction of remission of cancer with chemotherapy, or after autologous or allogeneic hematopoietic stem cell transplantation. In some embodiments, compositions described herein are administered to an individual in need of increasing the production of interferon gamma (IFNγ), TNF-α, and/or interleukin-2 (IL-2) in the treated subject relative to the production of these molecules in subjects who have not been administered one of the therapeutic compositions disclosed herein.

An effective amount of the compositions described herein, e.g., CARs, nucleic acids, recombinant cells, cell cultures, and/or pharmaceutical compositions, is determined based on the intended goal, for example tumor regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each individual. Frequency of administration could range from 1-2 days, to 2-6 hours, to 6-10 hours, to 1-2 weeks or longer depending on the judgment of the practitioner.

In some embodiments, administration is by bolus injection. In some embodiments, administration is by intravenous infusion. In some embodiments, a composition is administered is administered in a dosage of about 100 ng/kg of body weight per day to about 100 mg/kg of body weight per day. In some embodiments, a composition as disclosed herein is administered in a dosage of about 0.001 mg/kg to 100 mg/kg of body weight per day. In some embodiments, the therapeutic agents are administered in a single administration. In some embodiments, therapeutic agents are administered in multiple administrations, (e.g., once or more per week for one or more weeks).

One of ordinary skill in the art would be familiar with techniques for administering compositions of the disclosure to an individual. Furthermore, one of ordinary skill in the art would be familiar with techniques and pharmaceutical reagents necessary for preparation of these compositions prior to administration to an individual.

In certain embodiments of the present disclosure, the composition of the disclosure contains an aqueous composition that includes one or more of the CARs, nucleic acids, recombinant cells, cell cultures, and/or pharmaceutical compositions as described herein. Aqueous compositions of the present disclosure contain an effective amount of a composition disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the recombinant cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

One of ordinary skill in the art would appreciate that biological materials should be extensively dialyzed to remove undesired small molecular weight molecules and/or lyophilized for more ready formulation into a desired vehicle, where appropriate. The compositions described herein, e.g., CARs, nucleic acids, recombinant cells, cell cultures, and/or pharmaceutical compositions, can then generally be formulated for administration by any known route, such as parenteral administration. Determination of the amount of compositions to be administered can be made by one of skill in the art, and can in part be dependent on the extent and severity of cancer, and whether the recombinant cells are being administered for treatment of existing cancer or prevention of cancer. The preparation of the compositions containing the CARs, nucleic acids, recombinant cells, cell cultures, and/or pharmaceutical compositions of the disclosure can be known to those of skill in the art in light of the present disclosure.

Upon formulation, the compositions of the disclosure can be administered in a manner compatible with the dosage formulation and in such amount as is therapeutically effective. The compositions can be administered in a variety of dosage forms, such as the type of injectable solutions described above.

In some embodiments, CARs, nucleic acids, recombinant cells, cell cultures, and/or pharmaceutical compositions described herein can be used to reduce T cell exhaustion in the corresponding T cells or in the treated subject relative to a subject who has not been administered one of the therapeutic compositions disclosed herein. In some embodiments, CARs, nucleic acids, recombinant cells, cell cultures, and/or pharmaceutical compositions described herein can be used to stimulate proliferation and/or killing capacity of CAR T-cells in the treated subject relative to the production of these molecules in subjects who have not been administered one of the therapeutic compositions disclosed herein. The production of interferon gamma (IFNγ), TNF-α, and/or interleukin-2 (IL-2) can be stimulated to produce up to about 20 fold, such as any of about 2 fold, 3 fold, 4 fold, 5 fold, 6 fold, 7 fold, 8 fold, 9 fold, 10 fold, 11 fold, 12 fold, 13 fold, 14 fold, 15 fold 16 fold, 17 fold, 18 fold, 19 fold, or 20 fold or higher compared to the production of interferon gamma (IFNγ), TNF-α, and/or interleukin-2 (IL-2) in subjects who have not been administered one of the therapeutic compositions disclosed herein.

### Administration of Recombinant Cells to a Subject

In some embodiments, the methods of the disclosure involve administering an effective amount or number of the recombinants cells provided here to a subject in need thereof. This administering step can be accomplished using any method of implantation delivery in the art. For example, the recombinant cells can be infused directly in the subject's bloodstream or otherwise administered to the subject.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, i.e., long-term engraftment.

In some embodiments, the delivery of a recombinant cell composition (e.g., a composition including a plurality of recombinant cells according to any of the cells described herein) into a subject by a method or route results in at least partial localization of the cell composition at a desired site. Modes of administration include, e.g., injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection.

### Kits

Also provided herein are various kits for the practice of a method described herein. In particular, some embodiments of the disclosure provide kits for the diagnosis of a condition in a subject. Some other embodiments relate to kits for the prevention of a condition in a subject in need thereof. Some other embodiments relate to kits for methods of treating a condition in a subject in need thereof. For example, provided herein, in some embodiments, are kits that include one or more of the CAR polypeptides, recombinant nucleic acids, recombinant cells expressing the CAR polypeptides, or pharmaceutical compositions as provided and described herein, as well as written instructions for making and using the same.

In some embodiments, the kits of the disclosure further include one or more means useful for the administration of any one of the provided CAR polypeptides, recombinant nucleic acids, recombinant cells expressing the CAR polypeptides, or pharmaceutical compositions to an individual. For example, in some embodiments, the kits of the disclosure further include one or more syringes (including pre-filled syringes) and/or catheters (including pre-filled syringes) used to administer any one of the provided CAR polypeptides, recombinant nucleic acids, engineered cells, or pharmaceutical compositions to an individual. In some embodiments, a kit can have one or more additional therapeutic agents that can be administered simultaneously or sequentially with the other kit components for a desired purpose, e.g., for diagnosing, preventing, or treating a condition in a subject in need thereof.

In some embodiments, a kit can further include instructions for using the components of the kit to practice the methods disclosed herein.

No admission is made that any reference cited herein constitutes prior art. The discussion of the references states what their authors assert, and the inventors reserve the right to challenge the accuracy and pertinence of the cited documents. It can be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives can be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### Example 1

### Materials and Methods Used in Examples

This Example describes materials and methods used in exemplary experiments presented herein.

Primary human samples. AML samples were obtained according to the Administrative Panel on Human Subjects Research Institutional Review Board (IRB)-approved protocols (Stanford IRB no. 18329 and 6453) after informed consent. Cord blood was purchased from the NY Blood Center.

Cell lines. Packaging cell line 293GP, TIME cells, and human AML cell line HEL-2 were purchased from ATCC. MOLM-13, NOMO-1, and OCI-AML3 cells were purchased from DSMZ. THP-1 cells stably transduced with GFP-luciferase and iHUVEC endothelial cells were from other scientists. iHUVEC-19 cells were generated by transducing iHUVEC cells with a lentivirus containing a truncated version of CD19, including only the extracellular and transmembrane domains. Transduced cells underwent flow cytometry-based sorting on a BD FACSAria™ II to isolate a uniform population of CD19+ iHUVEC cells. All cell lines were verified by short tandem repeat analysis and confirmed to be Mycoplasma negative by PCR.

Generation of retroviral supernatant. F11-derived scFvs were codon-optimized and custom synthesized by GeneArt (Invitrogen) in either light-heavy or heavy-light orientation and connected with a (G₄S)₃ linker sequence. The resulting product was subcloned into an MSGV-1 based retroviral backbone plasmid encoding the transmembrane and intracellular domains of CD28-CD28 or CD8α-4-1BB and the intracellular domain of CD3ζ, as previously described in Long et al. 4-1BB costimulation ameliorates T cell exhaustion induced by tonic signaling of chimeric antigen receptors. Nat Med. 2015; 21(6):581-590. Retroviral particles containing CD93-28z or CD93-BBz CAR were produced by transfecting 293GP packaging cells with the corresponding CAR plasmid and an RD 114 envelope plasmid DNA using Lipofectamine 2000 and harvesting supernatant at 48 and 72 h post-transfection.

Humanized anti-CD93 antibody generation. Monoclonal mouse anti-human CD93 antibodies were generated by immunization of mice using CD93-Fc fusion protein. Hybridomas were generated using standard protocols. Hybridomas were selected and supernatants from the resulting clones were screened by enzyme linked immunosorbent assay (ELISA) and fluorescent activated cell sorting (FACS). A mouse hybridoma clone, F11, was identified to produce a monoclonal antibody with specificity against human CD93. Heavy and light chain variable regions of F11 were cloned from the hybridoma using universal antibody primers. Multiple clones of each V gene product were sequenced to monitor PCR-induced errors. The nucleotide sequences of V_{H} and V_{L} of F11 were determined. In order to select human antibody frameworks (FRs) to be used as templates for CDR-grafting, the mouse F11 V_{L} and V_{H} regions were compared with those of human germline sequences. Human IGKV2D-29 and IGHV1-2 subgroups were used as the bases for F11 humanization. Amino acid positions in the FR regions that differ between F11 and IGKV2D-29/IGHV1-2 sequences and that may have influence in antigen binding were identified through molecular modeling. Exemplary F11 mouse-human chimeric antibody heavy chain variable domain (V_{H}) can be found in SEQ ID NO: 1, containing the CDR H1 (SEQ ID NO: 3), the CDR H2 (SEQ ID NO: 4), and the CDR H3 (SEQ ID NO: 5). Exemplary F11 mouse-human chimeric antibody light chain variable domain (V_{H}) can be found in SEQ ID NO: 6, containing the CDR L1 (SEQ ID NO: 8), the CDR L2 (SEQ ID NO: 9), and the CDR L3 (SEQ ID NO: 10). Exemplary F11 humanized antibody heavy chain variable domain (V_{H}) can be found in SEQ ID NO: 2. Exemplary F11 humanized antibody light chain variable domain (V_{H}) can be found in SEQ ID NO: 7.

CAR T cell transduction. Buffy coats were purchased from Stanford Blood Center under an IRB-exempt protocol, and processed using Lymphoprep density gradient medium and SepMate-50 tubes following manufacturer's instructions. Primary human T cells were positively selected using the RosetteSep Human T cell Enrichment kit (Stem Cell Technologies), and cryopreserved at 1-2×10⁷ cells/vial in CryoStor CS10 cryopreservation medium (Stem Cell Technologies). Cryopreserved cells were thawed and activated the same day with CD3/CD28 Dynabeads (Gibco) at a 3:1 bead:cell ratio in T cell media (RPMI 1640 supplemented with 10% FBS, 10 mM HEPES, 2 mM GlutaMAX, 100 U/ml penicillin, 100 μg/ml streptomycin, and 100 IU/ml IL-2). Activated T cells were retrovirally transduced with CD93-CAR or co-transduced with CD93-CAR and CD19 iCAR on days 3 and 4 on Retronectin (Takara) coated plates, and anti-CD3/CD28 beads were removed on day 5. Media and IL-2 were changed every 2-3 days until day 10 or 11, when T cells were used for assays.

Cytokine production. 0.5-1×10⁵ tumor, CD34+, or endothelial cells and CAR T cells at effector to target (E:T) ratios between 1:4 and 2:1 were incubated at 37° C. in complete RPMI (RPMI 1640 supplemented with 10% FBS, 10 mM HEPES, 2 mM GlutaMAX, 100 U/ml penicillin, and 100 μg/ml streptomycin for 18-24 h in triplicate for each condition). Culture supernatants were collected and analyzed for IFNγ and IL-2 by ELISA (BioLegend) per manufacturer instructions.

IncuCyte™ Lysis Assay. 1×10⁵ GFP positive tumor cells and CAR T cells at E:T ratios of 1:8-1:1 were co-cultured in 200 μl complete RPMI at 37° C. for up to 72 h in triplicate for each condition. Plates were analyzed every 3 h using the IncuCyte ZOOM Live-Cell analysis system (Essen Bioscience). Four images per well at 10× zoom were collected at each time point. Total integrated GFP intensity per well was measured. Values were normalized to the starting measurement and plotted over time.

Mice. Immunocompromised NOD/SCID/IL2Rγ^{-/-} (NSG) mice were purchased from JAX and bred in house. All mice were bred, housed, and treated under Stanford University IACUC (APLAC) approved protocols. Six to eight week old mice were injected via tail vein with 1×10⁶ THP-1 cells stably expressing luciferase or SU555 patient-derived AML cells. Prior to CAR T injection, mice were assigned to groups to equalize pre-treatment leukemic burden, either by luminescence values or percent engraftment. CAR T cells were injected via tail vein at a time and dose provided in the figure legends. In the THP-1 model, leukemia progression was measured by bioluminescence using the IVIS imaging system and analyzed with Living Image software. In the PDX model, leukemia progression was measured by phenotypic analysis of bone marrow (BM) aspirates, performed every 3-5 weeks on alternating femurs. At least 5 mice per group were treated in each experiment and each experiment was repeated 2-3 times as indicated in the figure legend.

Flow cytometry. All samples were analyzed with an LSR Fortessa or FACSAria II (BD Bioscience) and data were analyzed with FlowJo software. Fc block was used preceding staining of all myeloid cells (Clone 3070, BD Biosciences). CD93-CARs were detected with CD93-Fc chimera protein (R&D) and CD19 iCARs were detected with anti-CD19-CAR idiotype (Jena et al., Chimeric antigen receptor (CAR)-specific monoclonal antibody to detect CD19-specific T cells in clinical trials. PLoS One. 2013; 8(3):e57838) after conjugation to DyLight 650 or Dylight 488, respectively, with an Antibody Labeling Kit (ThermoFisher Scientific). T cell phenotype was evaluated with the following antibodies: CD4-BUV395 (Clone SK3, BD Biosciences), CD8-BUV805 (Clone SK1, BD Biosciences), PD-1 (clone eBioJ105, eBioscience), Tim-3-BV510 (clone F38-2E2, BioLegend), LAG-3-PE (clone 3DS223H, eBioscience), CD39-FITC (Clone A1, BioLegend). CD93 was detected on tumor or endothelial cells using either the F11 antibody or CD93-APC (clone AA4.1, Biolegend). Other antibodies used to phenotype AML cells and endothelial cells include: CD45-PerCp/Cy5.5 (Clone HI30, eBioscience), CD33-BV421 (Clone WM53, BD Biosciences), CD123-BV711 (Clone 7G3, BD Biosciences), CD38-PE (Clone HB7, BD Biosciences). Fixable viability stain 780 (BD Biosciences) was used to exclude dead cells from further phenotypic analyses. Antibodies for phenotypic analysis of hematopoietic cells isolated from CD34+ cells include: CD45RA-Qdot605 (BioLegend), CD90-FITC (BD Biosciences), CD123-PE (BD Biosciences), CD38-PE-Cy7 (BD Biosciences), CD34-APC (BD Biosciences), CD10-APC-Cy7 (BioLegend).

Blood analysis. Peripheral blood sampling from the mice was collected on day 14 after CAR T cell injection to measure CAR T expansion. Fifty microliter of blood was labeled with CD45, CD3, CD4, and CD8 followed by lysis with BD FACS Lysing Solution. Results were quantified using CountBright Absolute Counting Beads (ThermoFisher) on a BD Fortessa flow cytometer.

Methylcellulose colony forming assay. Cord blood was obtained from the New York Blood Center with informed consent according to protocols approved by the Stem Cell Research Oversight Panel (SCRO-291). CD34+ cells were enriched from FICOLL-separated mononuclear cells with the CD34 MicroBead kit for Humans (Miltenyi Biotec, 130-046-703) according to manufacturer instructions. After incubation without CAR or with mock-transduced or CD93-CAR T cells for 24 h, CD34+ cells were sorted on a FACS Aria II (BD) and re-suspended in 400 μl IMDM with 4 ml MethoCult H4435 (STEMCELL Technologies), according to the manufacturer's instructions, and plated in triplicate in 6-well SmartDish (STEMCELL Technologies) plates at 1×10³ cells per well. Plates were incubated at 37° C. and 5% CO₂ for 14 d. After 14 days, colonies were scored on an inverted microscope (brand, magnification) for the quantity of erythroid (BFU-E/CFU-E) or granulocyte-macrophage (CFU-G/M/GM) colonies.

Immunohistochemistry (IHC) and scoring. CD93 expression was assessed using the Ventana DISCOVERY Ultra autostainer (Ventana Medical Systems, Tucson, Arizona). Baked and deparaffinized FFPE tissue sections were incubated in Tris-based buffer (CC1, Ventana) at 95° C. for 64 min for antigen retrieval, followed by incubation at room temperature for 1 hour with CD93 primary antibody (Clone AA4.1, 1:50, ThermoFisher) prepared in DISCOVERY Ab diluent (Ventana). Bound antibodies were visualized with the UltraMap DAB Detection Kit (Ventana). Stained samples were digitized with Leica SCN400 scanner (Leica Microsystems; Concord, Ontario, Canada) at magnification equivalent to 20×. The images were subsequently stored in the SlidePath Digital Imaging Hub (DIH; Leica Microsystems) at the Vancouver Prostate Centre. For IHC scoring, staining intensity was assigned via a four-point scale system (descriptively, 0=no staining, 1=low, but detectable degree of staining, 2=clearly positive cases, and 3=strong expression) and percentage of positive cells (0 to 100%) was also determined. IHC score was then calculated per sample as staining intensity multiplied by percentage of positive cells. The average H-score was calculated for each tissue type.

Analysis of single-cell RNA-sequencing data. Processed scRNA-seq data (cell by gene counts table) for human lung were downloaded from Synapse (accession #syn21041850) (Travaglini et al., A molecular cell atlas of the human lung from single cell RNA sequencing. bioRxiv. 2019:742320). These data were analyzed using Scanpy v.1.4.3. First, low-quality cells containing fewer than 250 genes, fewer than 500 counts, or more than 25% mitochondrial reads, were removed. Counts were depth-normalized to a sum of 10,000 per cell, and then log-transformed with a pseudocount of 1. The top 2000 variable genes were identified with sc.pp.highly_variable_genes, and effects due to the number of genes detected per cell were regressed out. Data were scaled with max_value=10, and PCA was performed with default settings. For sc.pp.neighbors, the parameters n_neighbors=10, n_pcs=50, random_state=1 were provided, and for Leiden clustering was performed with resolution=0.1, and random_state=1. Gene expression was then visualized on the UMAP projection as the log depth-normalized counts. The sc.tl.rank_genes_group function was used to identify marker genes for each cluster with method=‘logreg’, which allowed classification of the endothelial sub-types present in this dataset. For violin plots, gene expression was visualized as depth-normalized counts (not log-transformed). Processed scRNA data for human pancreas were downloaded from the Genome Sequence Archive (project #PRJCA001063) (Peng et al., Single-cell RNA-seq highlights intratumoral heterogeneity and malignant progression in pancreatic ductal adenocarcinoma. Cell Res. 2019; 29(9):725-738.). The data were first subset to include only those samples generated from healthy (non-tumor) samples, and each sample was processed using Scrublet to remove any potential doublets, with the settings min_counts=2, min_cells=3, min_gene_variability_pct1=85, n_prin_comps=30, threshold=0.2. Data were then processed in Scanpy using the same workflow as above, with the exception of regressing out batch effects due to both the number of genes detected and the individual sample/patient, and using a resolution of 0.15 for Leiden clustering.

Bulk RNA-seq. Bulk RNA was isolated from three AML cell lines (Kasumi-1, NOMO-1, and THP-1) and two endothelial cell lines (iH UVEC and TIME) after 24 h without treatment or after incubation with IFNγ and/or TNFα at 10 ng/ml. Total RNA was isolated from 2×10⁶ cells using Qiagen RNEasy isolation kit. Bulk RNA-seq was performed by Novogene (Sacramento, CA) using the Illumina Novaseq6000 platform, 150 bp paired-end reads, at 18-34 million reads per sample. Reads were quantified using Salmon (v1.2.0) (Patro et al., Salmon provides fast and bias-aware quantification of transcript expression. Nat Methods. 2017; 14(4):417-419) using the human transcriptome (GRCh38.p 13, Ensembl release 99) as the reference. Transcript annotation was performed with tximeta (v1.6.2) (Love et al., Tximeta: Reference sequence checksums for provenance identification in RNA-seq. PLoS Comput Biol. 2020; 16(2):e1007664) and differentially expressed genes were identified using DESeq2 (v1.28.1) (Love et al., Moderated estimation of fold change and dispersion for RNA-seq data with DESeq2. Genome Biol. 2014; 15(12):550). The variance stabilizing transformation was applied prior to performing principle component analysis with DESeq2. K means clustering and heatmapping were performed with pheatmap (v1.0.12) and Venn Diagrams were created with BioVenn.

Gene Set Enrichment Analysis and PSCAN analysis. Gene Set Enrichment Analysis was performed using GSEA software version 4.0.3 (Broad Institute). The “gene set” permutation type was used and 1000 permutations were performed. Gene collections were imported from the Molecular Signatures Database. PCAN analysis was performed on gene promoters spanning −450 to +50 base pairs flanking the transcriptional start site (Zambelli et al., Pscan: finding overrepresented transcription factor binding site motifs in sequences from co-regulated or co-expressed genes. Nucleic Acids Res. 2009; 37 (Web Server issue): W247-252).

Statistical analysis. Data were analyzed using GraphPad Prism software. Graphs represent individual values or group mean values±SEM for in vitro and in vivo experiments. P values were calculated with the statistical test described in the corresponding figure legend. P<0.05 was considered statistically significant, and P values are denoted with asterisks as follows (P>0.05: not significant, NS; P<0.05: *; P<0.01: **; P<0.001: ***; P<0.0001: ****).

Data Sharing Statement. The sequencing datasets generated in this publication have been deposited in NCBI Gene Expression Omnibus (GEO) (Barrett et al., NCBI GEO: archive for functional genomics data sets—update. Nucleic Acids Res. 2013; 41 (Database issue): D991-995; Edgar and Barrett, NCBI GEO standards and services for microarray data. Nat Biotechnol. 2006; 24(12):1471-1472), and are accessible through GEO with the accession number: GSE159991.

### Example 2

### Identifying CD93 as a Cell Surface Target in AML

This Example describes experiments performed to illustrate the identification of CD93 as a suitable target for CAR-based immunotherapy of human AML.

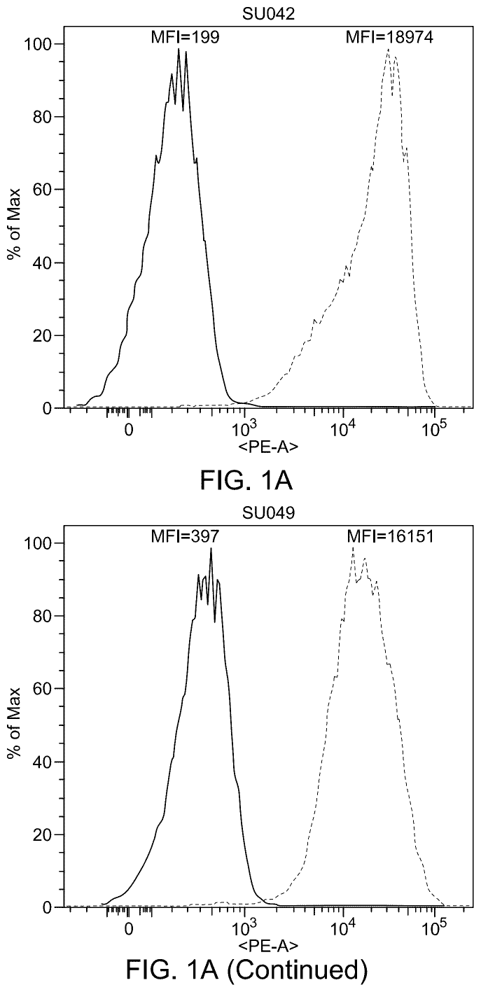

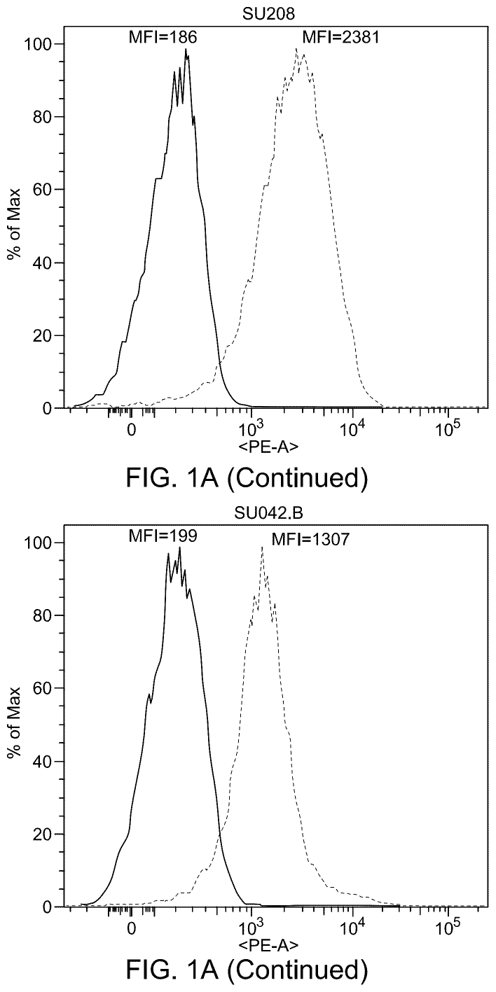

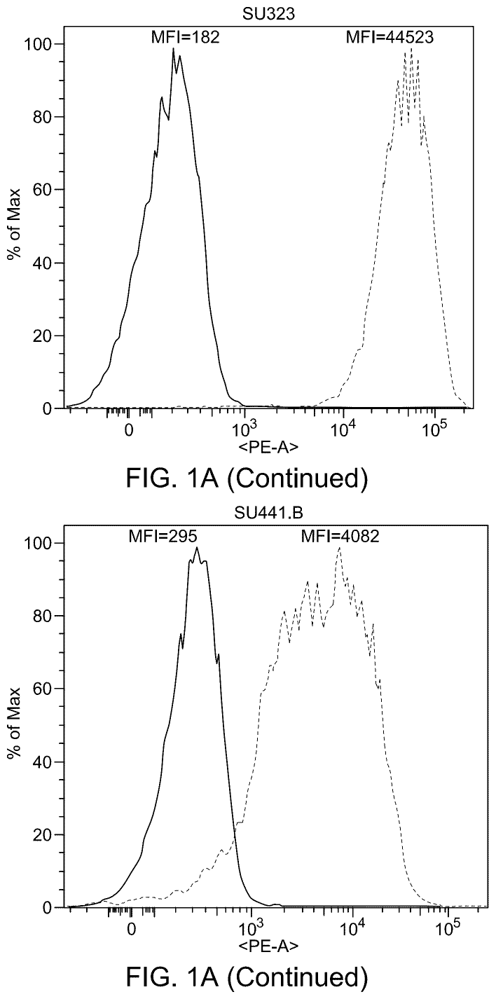

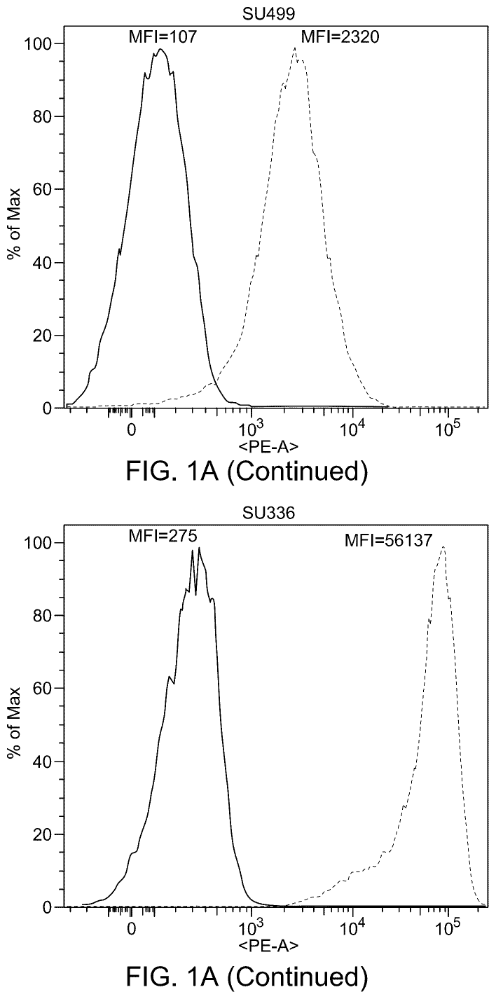

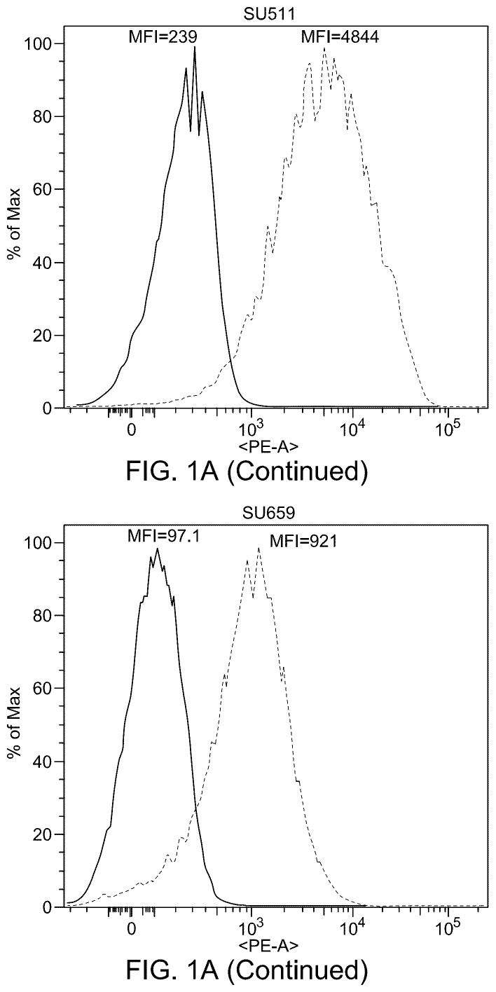

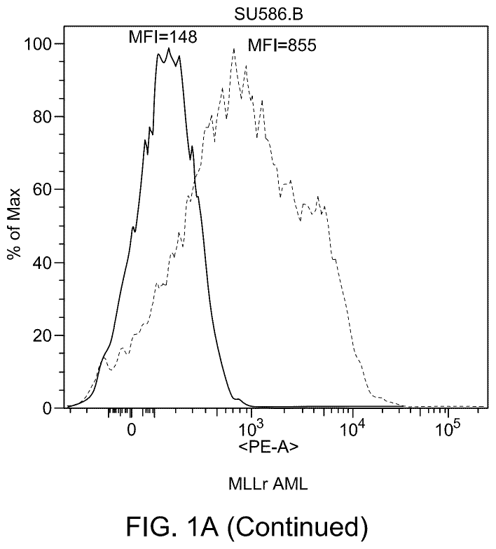

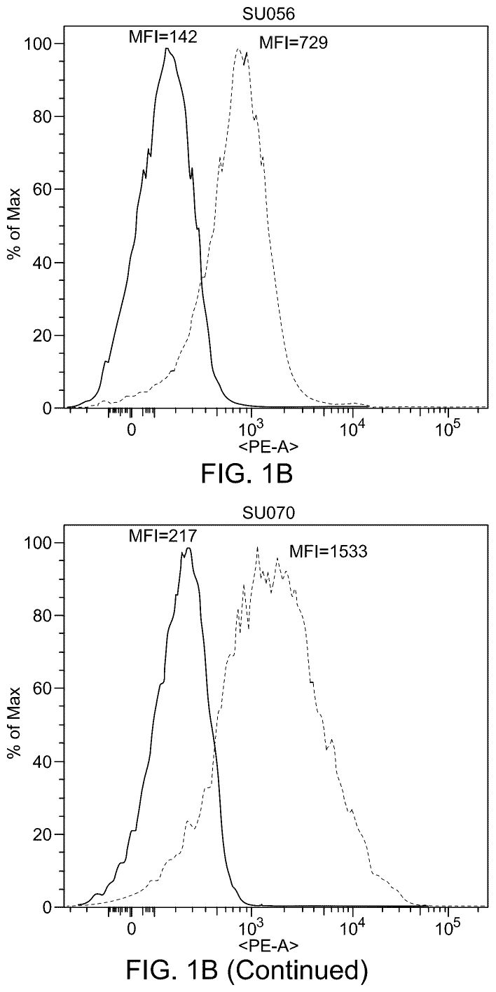

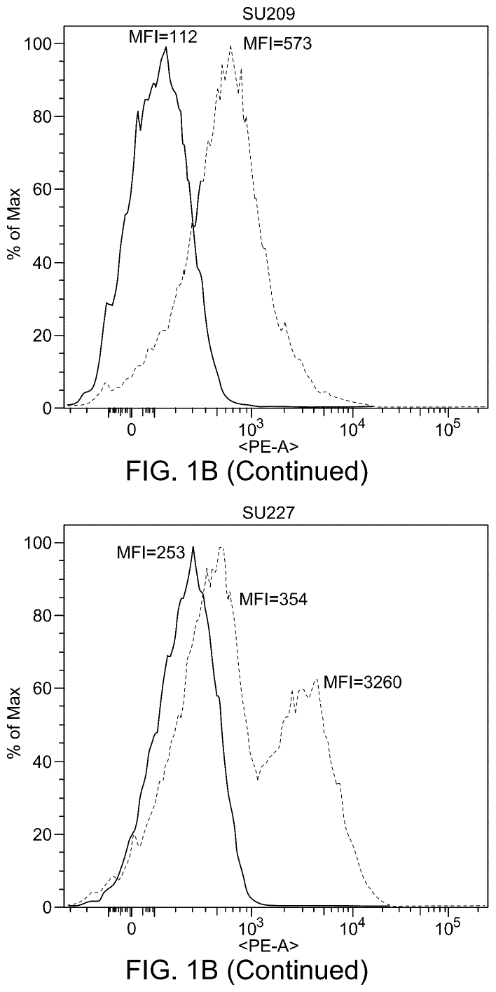

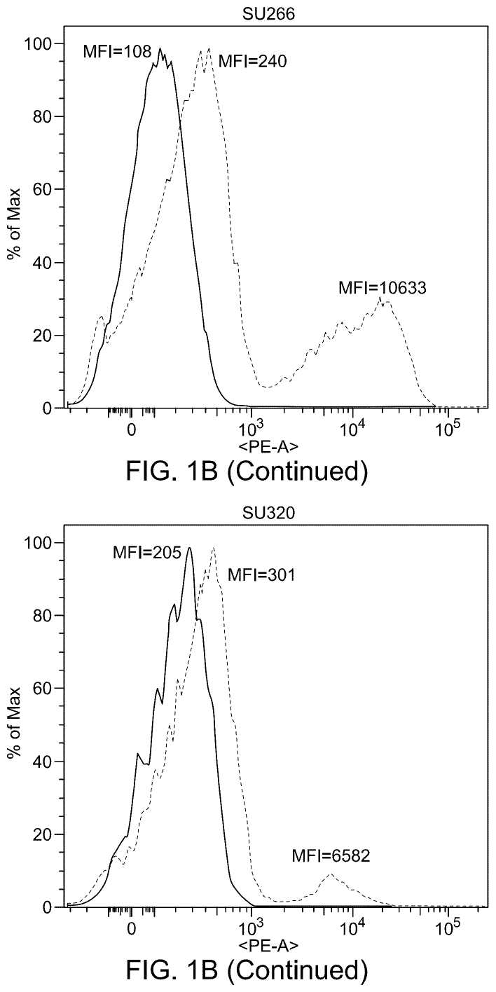

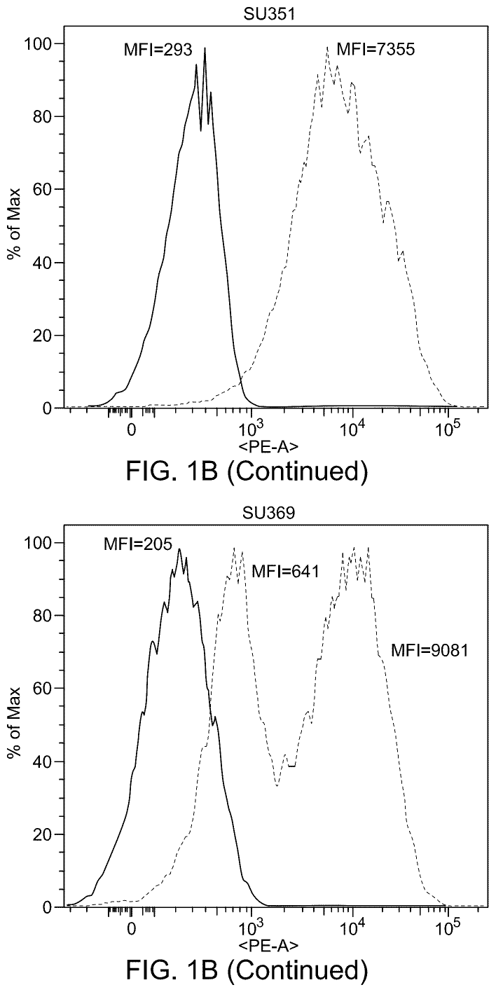

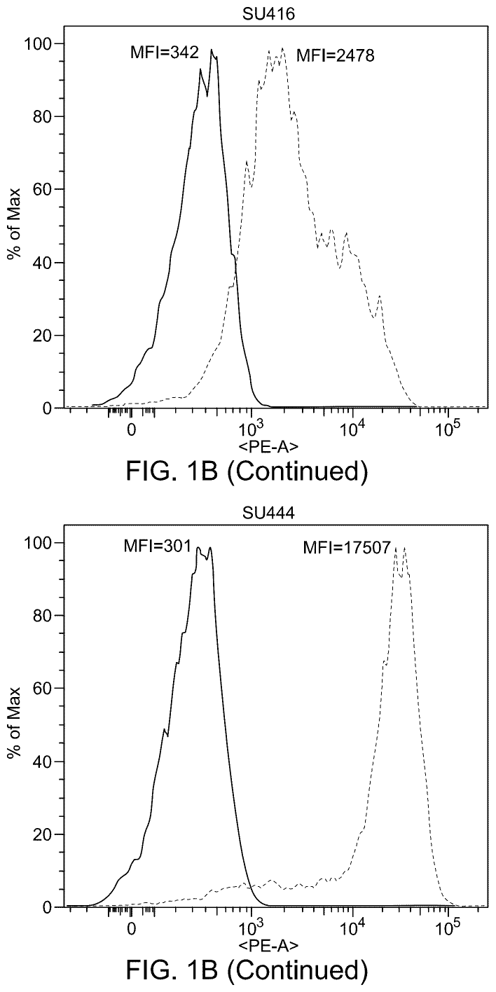

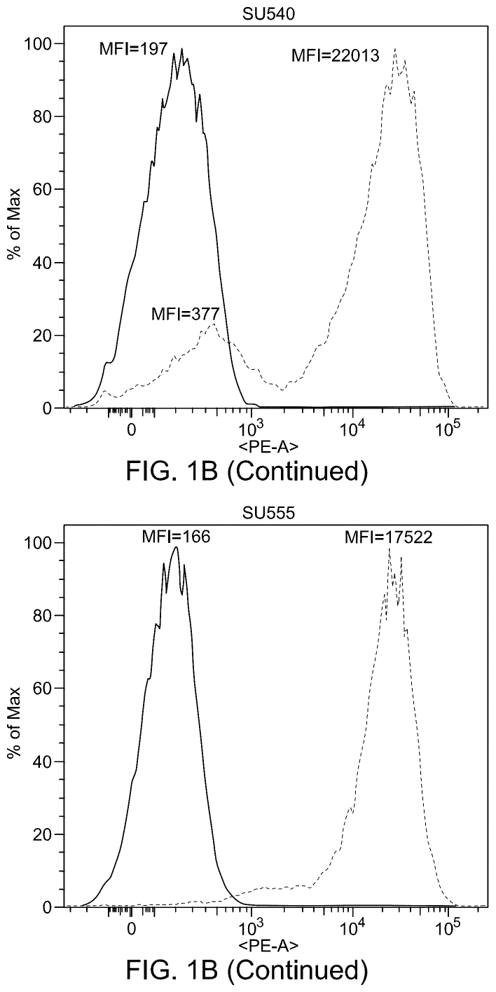

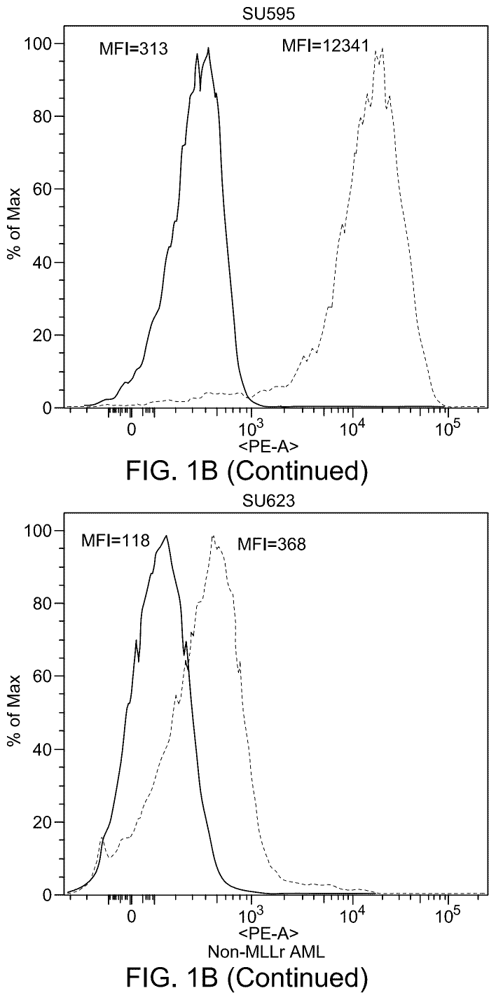

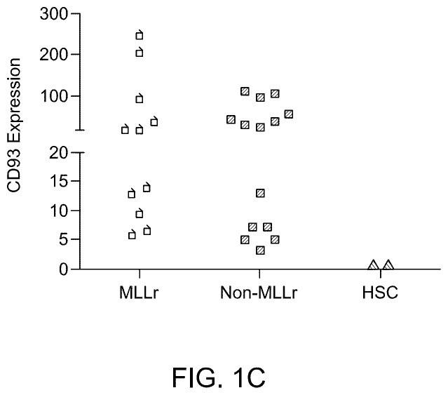

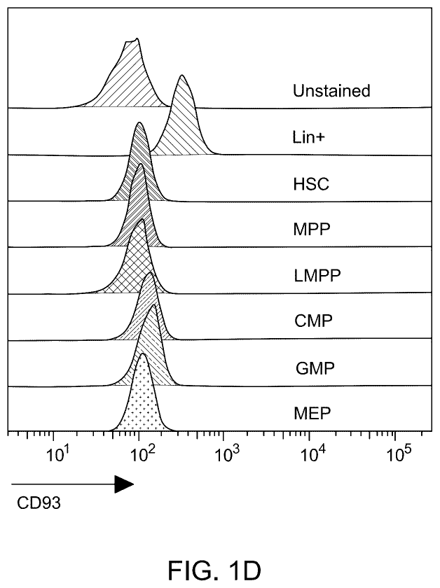

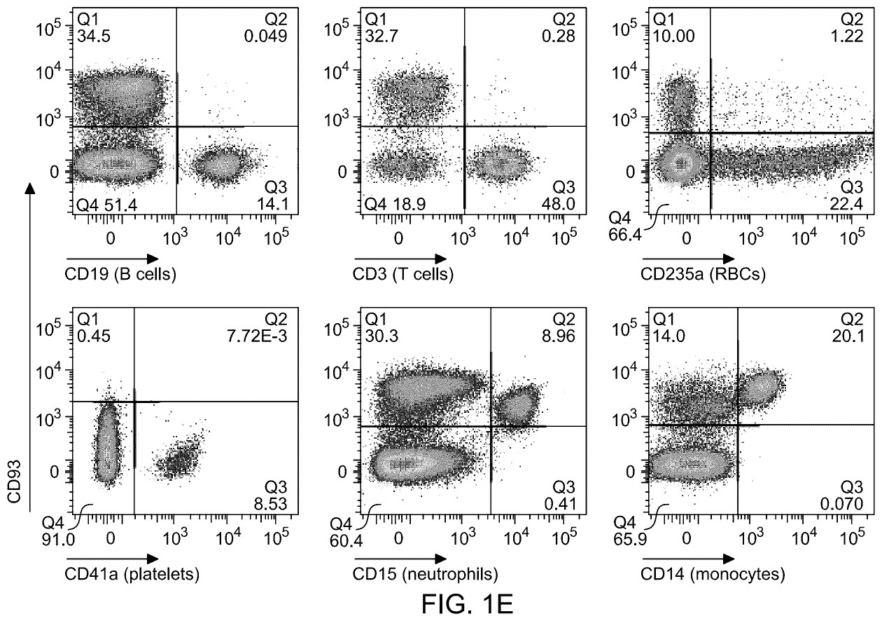

CD93 expression was detected by flow cytometry on the majority of primary AML samples, often uniformly and at high levels, including on both MLLr-AML and non-MLLr-AML (FIGS. 1A-1C). CD93 is not expressed on hematopoietic stem cells (HSCs) or any myeloid progenitor populations (FIG. 1D), but is expressed on mature myeloid cells including neutrophils and monocytes. CD93 is absent on lymphocytes, red blood cells, and platelets (FIG. 1E). The absence of expression on hematopoietic progenitors distinguishes CD93 from many previously studied AML targets including CD33, which is expressed on myeloid progenitors but not on HSCs (Griffin et al., A monoclonal antibody reactive with normal and leukemic human myeloid progenitor cells. Leuk Res. 1984; 8(4):521-534; Andrews et al., The L4F3 antigen is expressed by unipotent and multipotent colony-forming cells but not by their precursors. Blood 1986; 68(5):1030-1035; Robertson et al., Human bone marrow depleted of CD33-positive cells mediates delayed but durable reconstitution of hematopoiesis: clinical trial of MY9 monoclonal antibody-purged autografts for the treatment of acute myeloid leukemia. Blood 1992; 79(9):2229-2236), and CD123, which is expressed broadly on myeloid and lymphoid progenitors and on HSCs (Gill et al. Blood 2014; 123(15):2343-2354; Taussig et al., Hematopoietic stem cells express multiple myeloid markers: implications for the origin and targeted therapy of acute myeloid leukemia. Blood 2005; 106(13):4086-4092; Testa et al., Expression of growth factor receptors in unilineage differentiation culture of purified hematopoietic progenitors. Blood 1996; 88(9):3391-3406).

### Example 3

### CD93-CAR T Cells Mediate Antigen-Specific Effector Function and Cytotoxicity In Vitro Against AML Targets

This Example describes experiments performed to illustrate the capacity of CD93-CAR molecule to activate T cells and promote antigen-specific effector function and cytotoxicity of the T cells against AML target cells.

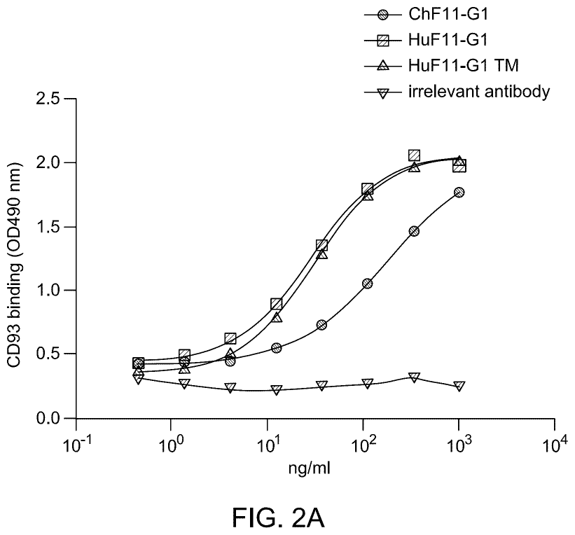

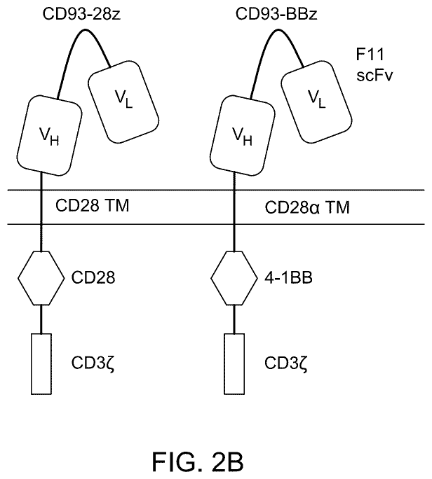

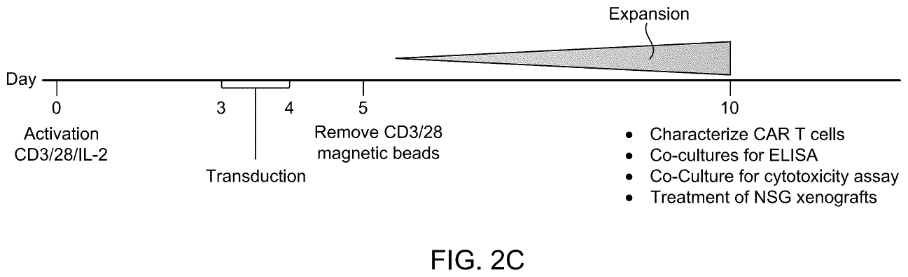

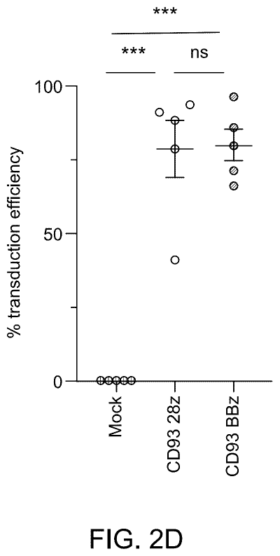

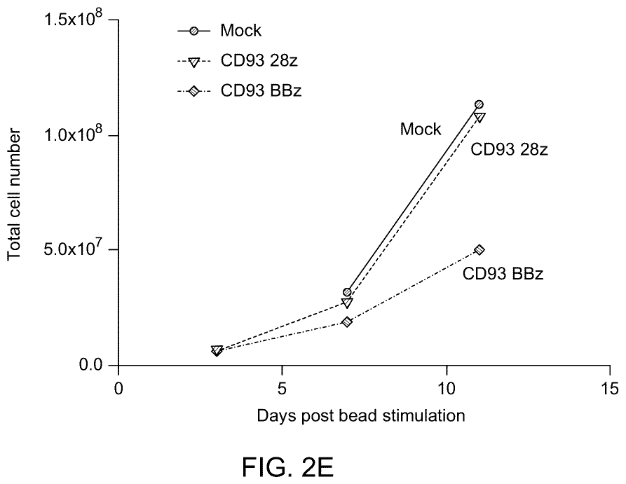

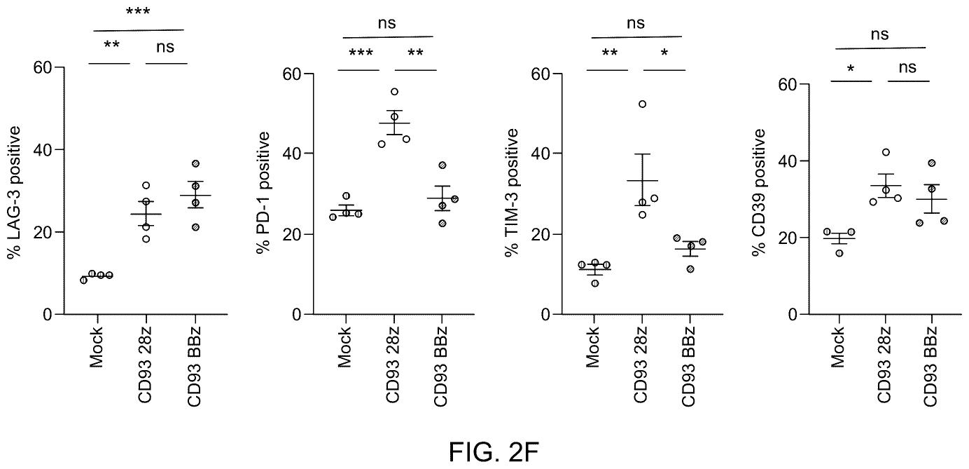

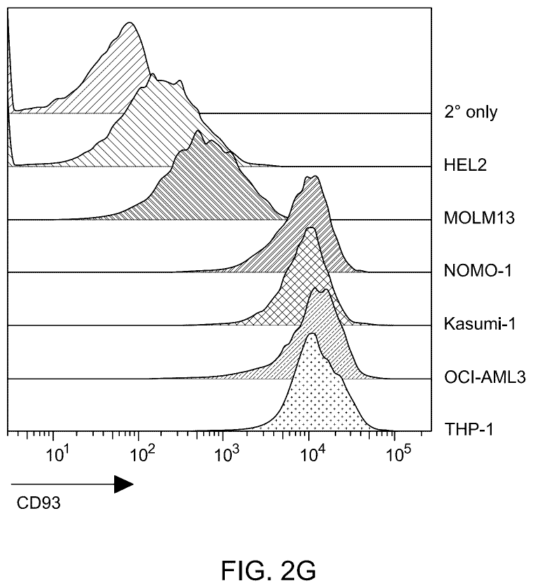

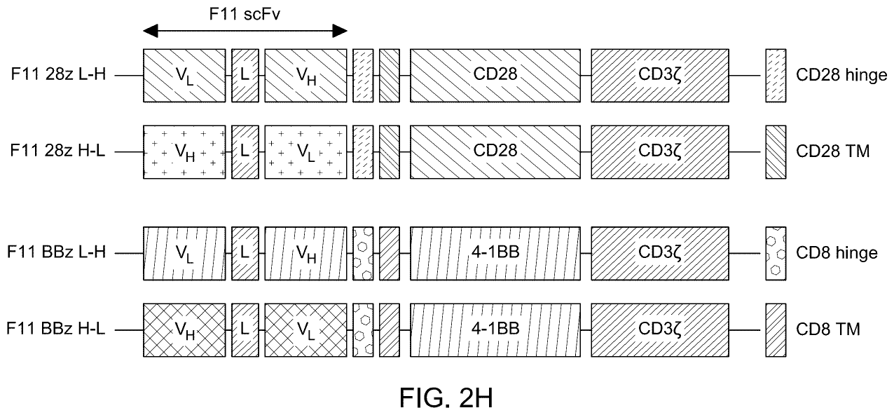

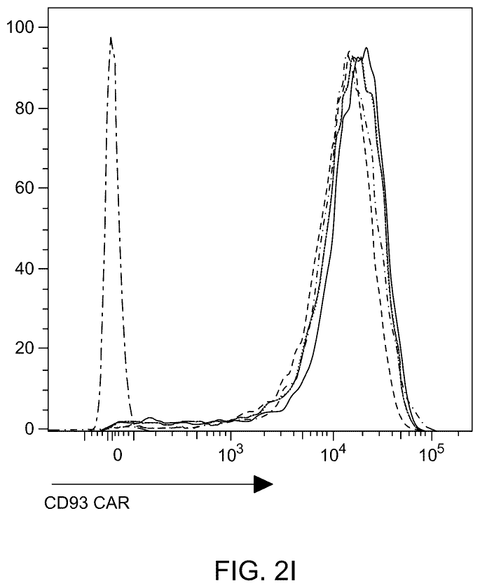

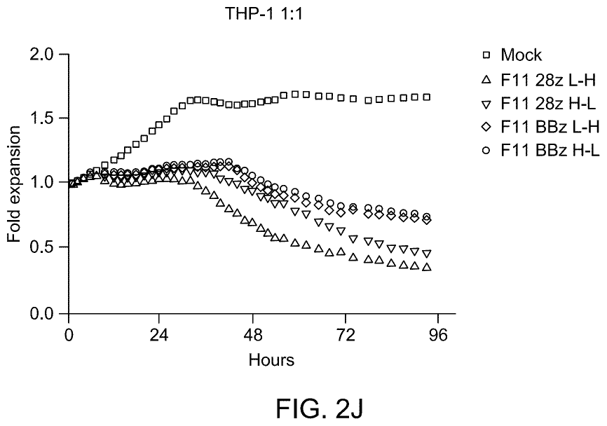

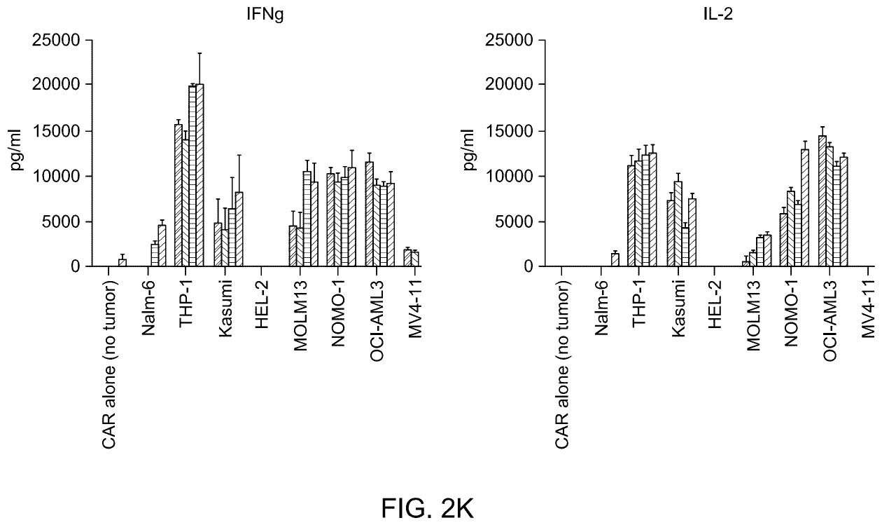

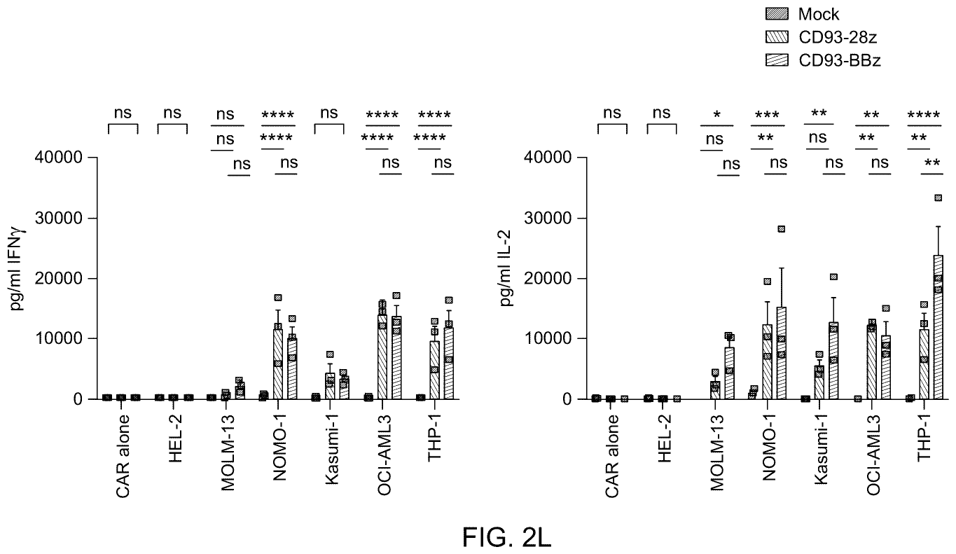

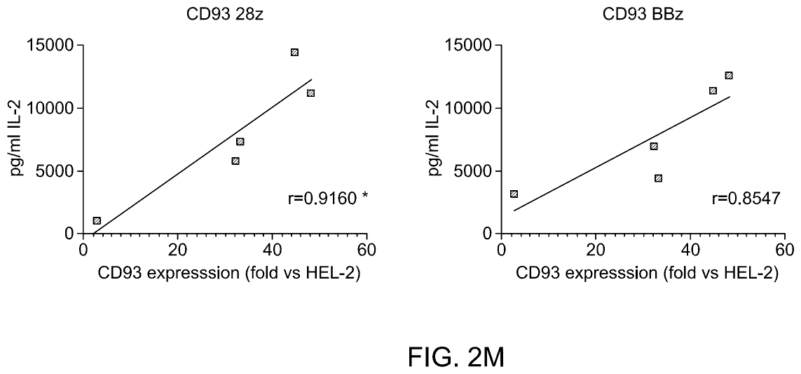

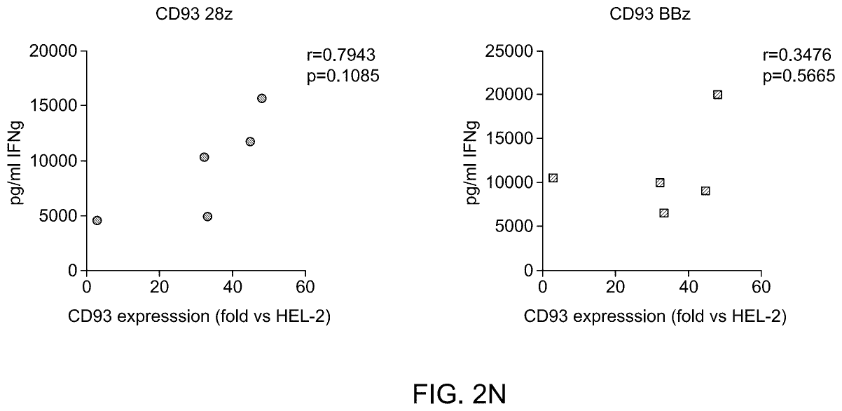

To redirect T cell specificity against CD93-expressing AML cells, retroviral vectors were generated to encode CD93-CAR polypeptides that incorporated a CD93-specific scFv derived from a humanized chimeric monoclonal antibody (F11), previously described in WO2020/180706. The binding of the chimeric F11 antibody or the humanized F11 antibody to human 93 is shown in FIG. 2A. Second generation CARs were constructed using codon-optimized sequences encoding the F11 scFv at the N-terminus with light and heavy chains connected through a (G₄S)₃ linker, and fused to either a CD28 hinge-transmembrane domain, a CD28 costimulatory endodomain and CD3ζ (CD93-28z) or to a CD8a hinge-transmembrane domain, a 4-1BB costimulatory endodomain and CD3ζ (CD93-BBz) (FIG. 2B). Exemplary F11 mouse-human chimeric scFv full-length sequences, with the (G₄S)₃ linker, can be found in SEQ ID NOs: 12 (the H-L format) and 13 (the L-H format). Exemplary F11 humanized scFv full-length sequences, with the (G₄S)₃ linker, can be found in SEQ ID NOs: 14 (the H-L format) and 15 (the L-H format). Primary T cells activated and transduced with CD93-28z or CD93-BBz CAR expanded 30-50-fold in culture with consistent CAR transduction efficiency of >75% and with comparable MFI (FIG. 2C-2E). Similar to previous reports (Long et al., Nat Med. 2015; 21(6):581-590), T cell exhaustion markers PD-1 and TIM-3 were higher in CD93-28z CAR T cells compared to CD93-BBz CAR T cells (FIG. 2F).

To evaluate CD93-CAR T cell functions in vitro, cytokine production and cytotoxicity of these cells were analyzed after co-culturing with AML cell lines with varying expression levels of CD93 (FIG. 2G). Of note, the orientation of the light and heavy chain of the scFv did not impact CAR T cell efficacy in vitro (FIGS. 2H-2K). CD93-CAR T cells produced minimal cytokines at baseline, but secreted IFNγ and IL-2 upon recognition of CD93 expressing AML cells, in contrast to mock-transduced T cells (FIGS. 2K-2L). Previous reports emphasized the importance of target antigen density, see, e.g., Caruso et al., Tuning Sensitivity of CAR to EGFR Density Limits Recognition of Normal Tissue While Maintaining Potent Antitumor Activity. Cancer Res. 2015; 75(17):3505-3518; Fry et al., CD22-targeted CAR T cells induce remission in B-ALL that is naïve or resistant to CD19-targeted CAR immunotherapy. Nat Med. 2018; 24(1):20-28; Liu et al., Affinity-Tuned ErbB2 or EGFR Chimeric Antigen Receptor T Cells Exhibit an Increased Therapeutic Index against Tumors in Mice. Cancer Res. 2015; 75(17):3596-3607; Ramakrishna et al., Modulation of Target Antigen Density Improves CAR T-cell Functionality and Persistence. Clin Cancer Res. 2019; 25(17):5329-5341; Walker et al., Tumor Antigen and Receptor Densities Regulate Efficacy of a Chimeric Antigen Receptor Targeting Anaplastic Lymphoma Kinase. Mol Ther. 2017; 25(9):2189-2201; Watanabe et al., Target antigen density governs the efficacy of anti-CD20-CD28-CD3 zeta chimeric antigen receptor-modified effector CD8+ T cells. J Immunol. 2015; 194(3):911-920; Majzner et al., Tuning the Antigen Density Requirement for CAR T-cell Activity. Cancer Discov. 2020; 10(5):702-723. Similarly, in this experiment cytokine production was directly proportional to the intensity of CD93 staining on the surface of AML cells (FIGS. 2M-2N). CD93-CAR T cells also killed AML cells stably expressing GFP in an Incucyte™ cytotoxicity assay (FIG. 2O), while significant cytotoxicity can be seen as low as 1:8 E:T ratio.

### Example 4

### CD93-CAR T Cells Exert Potent Anti-Leukemic Effect In Vivo in Cell Lines and Patient Derived Xenograft Murine Models

This Example describes experiments performed to evaluate CD93-CAR T cells for their anti-cancer function in cell lines and animal models.

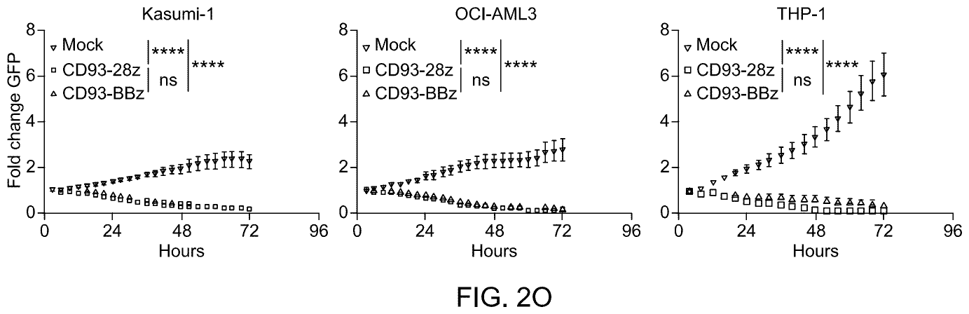

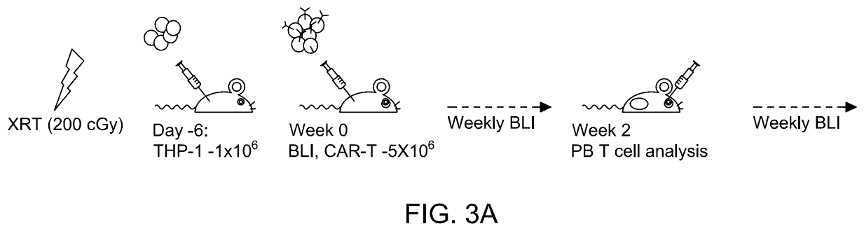

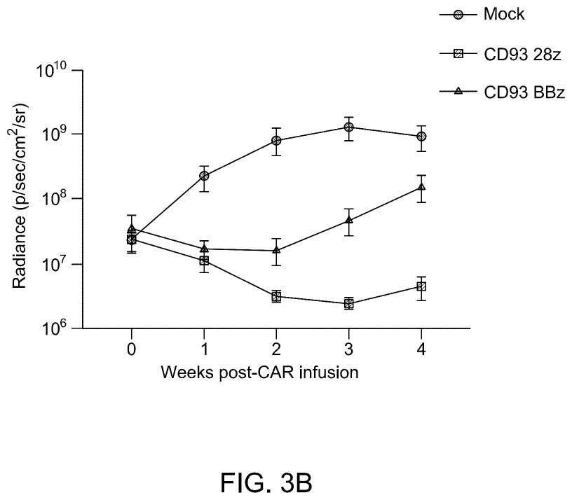

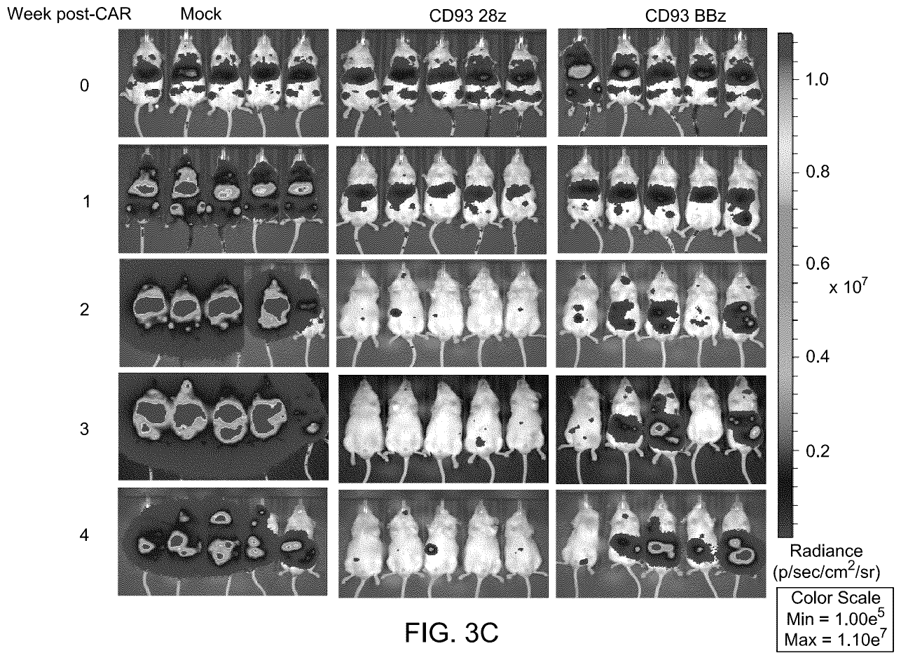

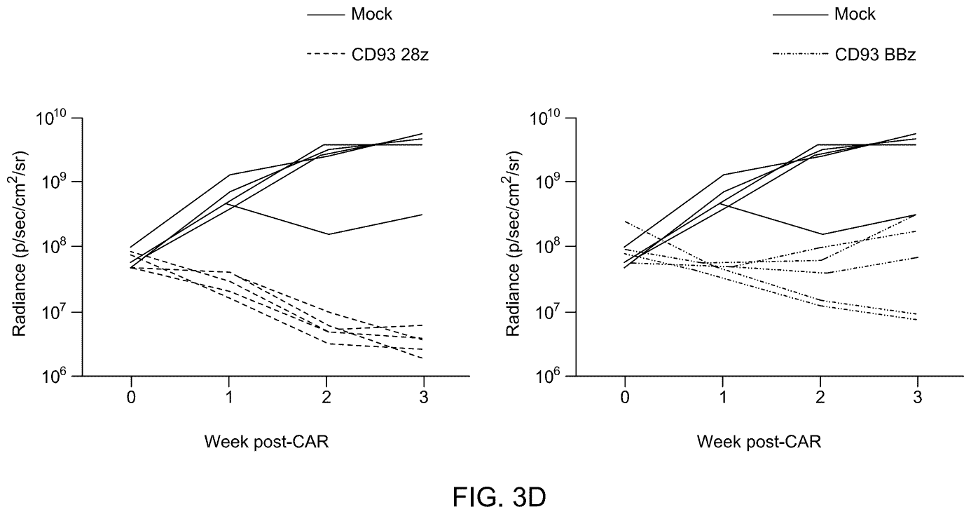

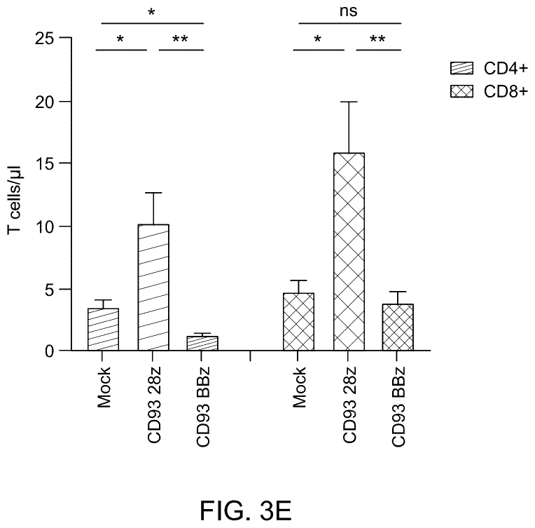

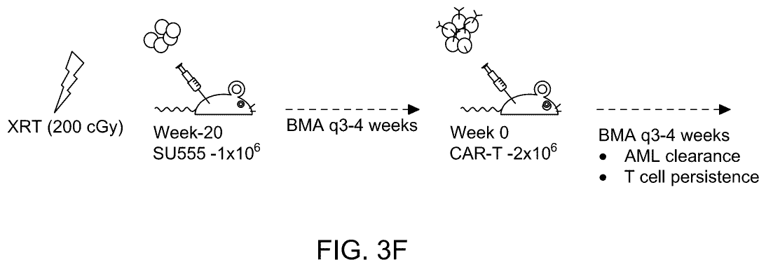

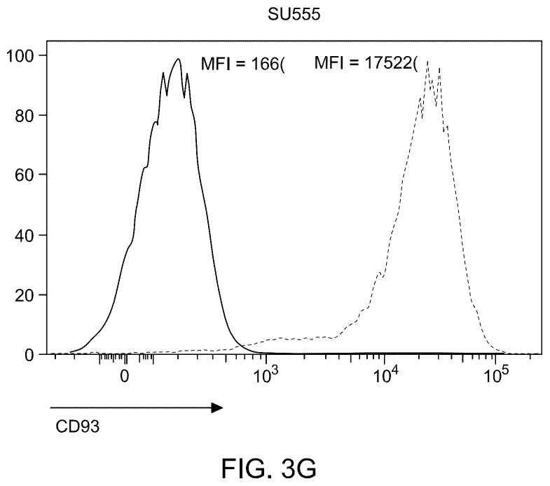

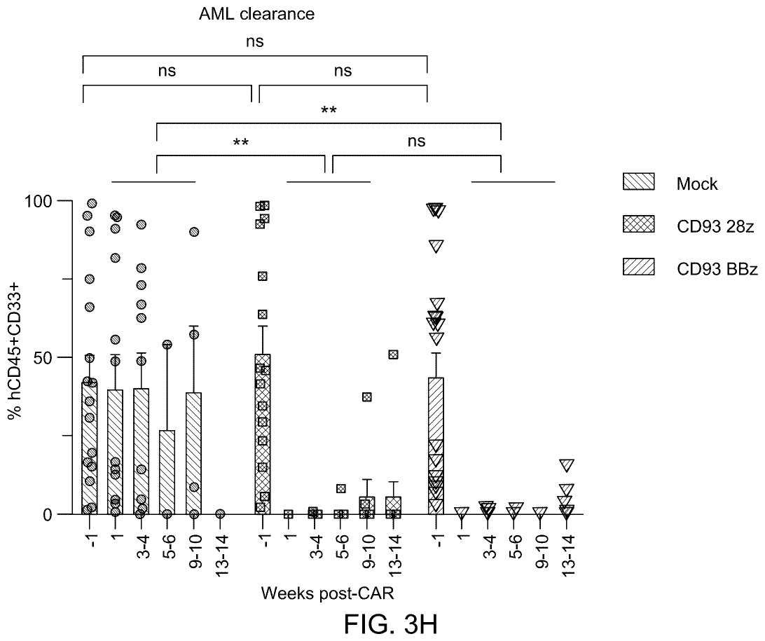

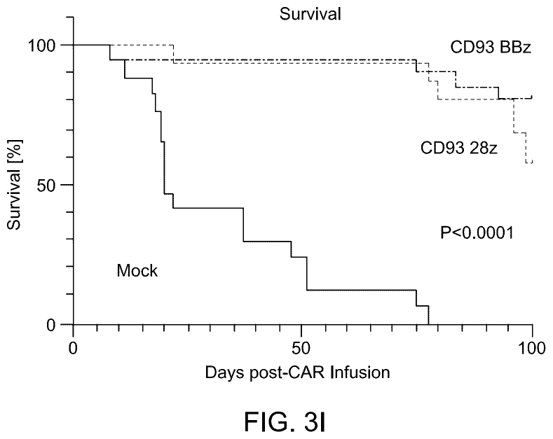

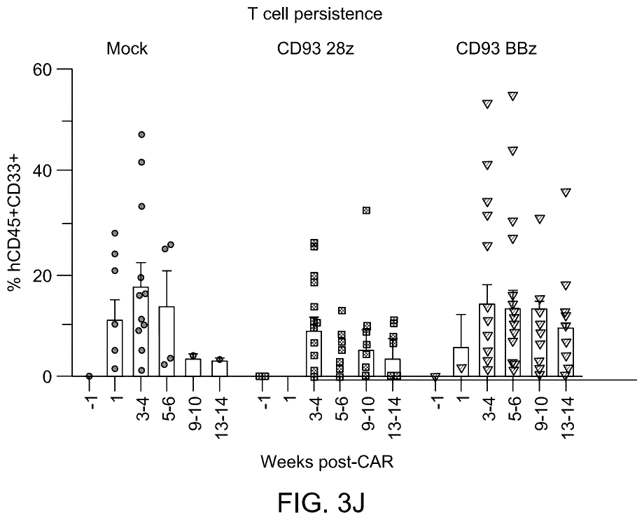

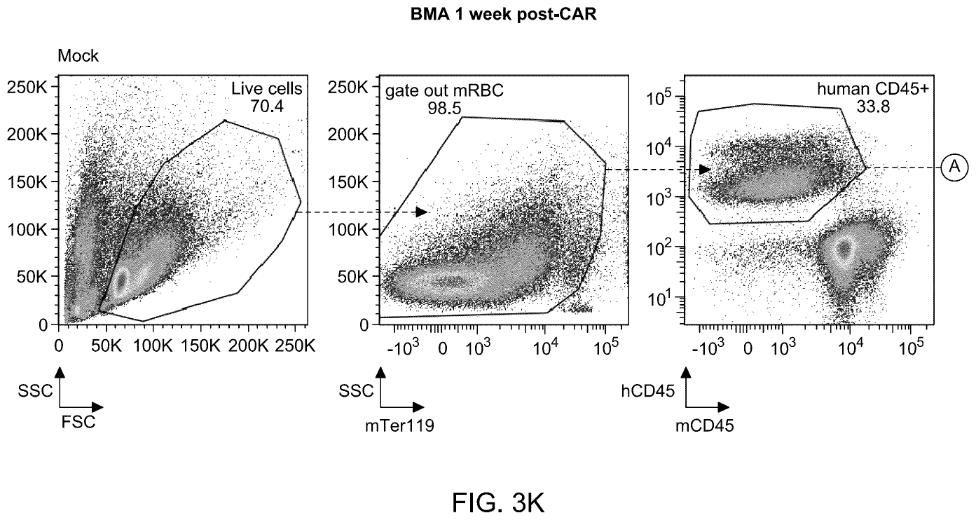

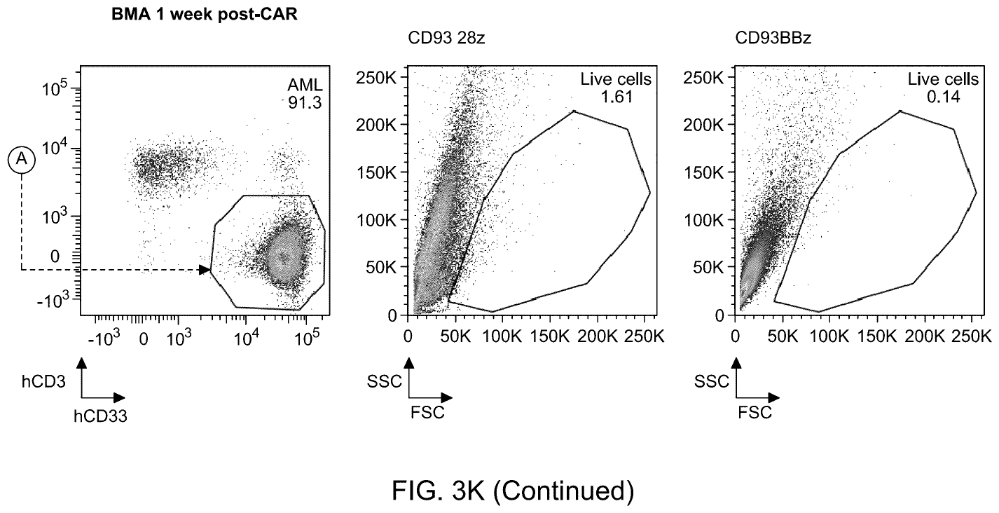

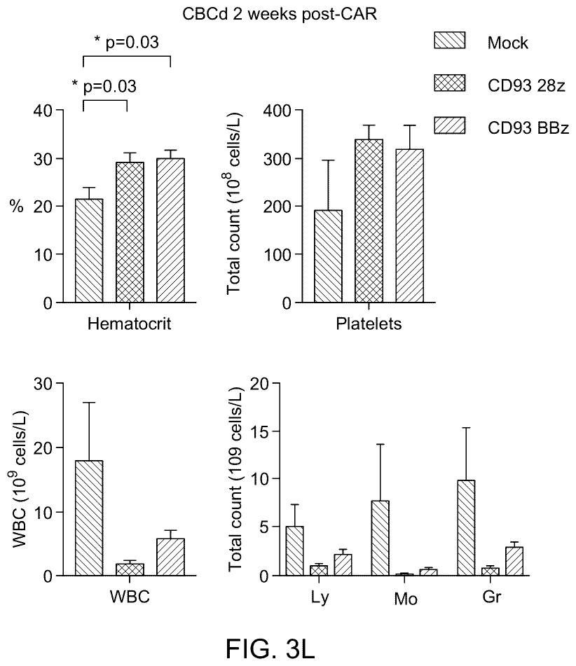

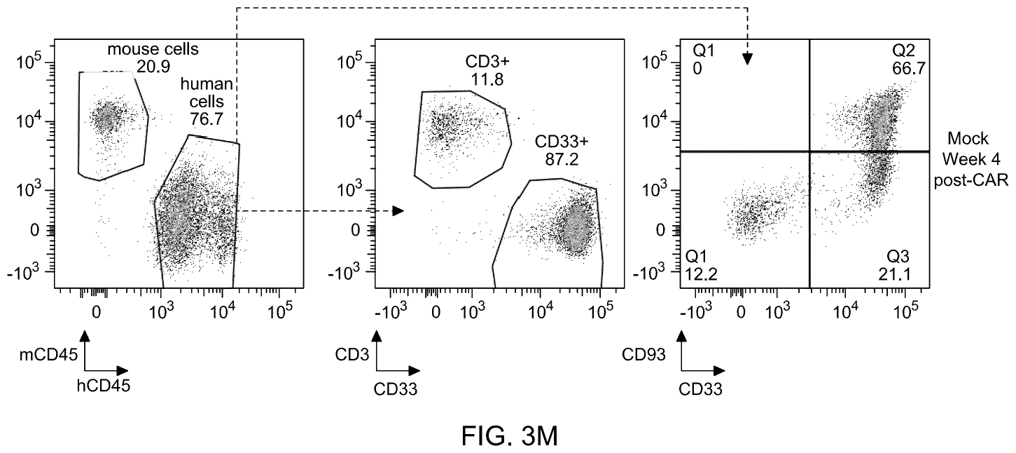

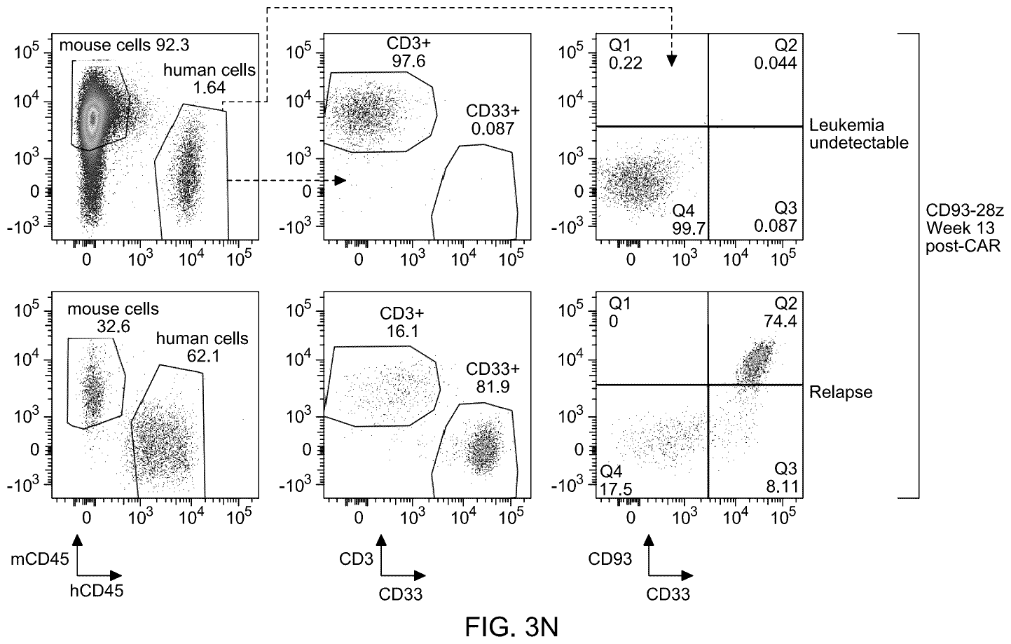

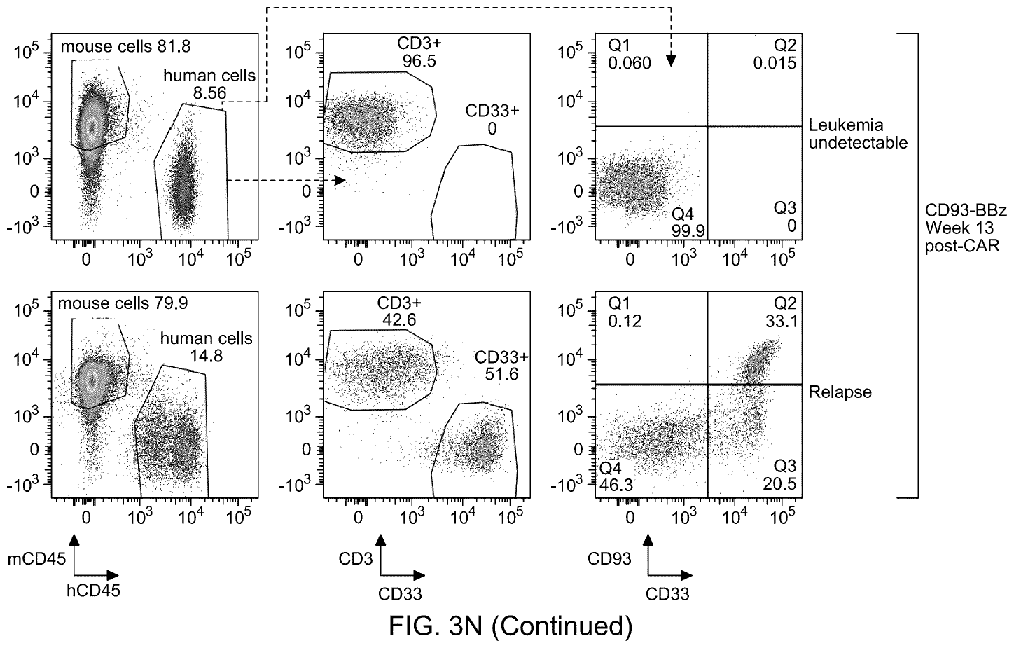

The in vivo efficacy of CD93-CAR T cells was evaluated in two murine xenograft models of human AML. NSG mice were sub-lethally irradiated and engrafted with luciferase-expressing THP-1 cells. Once engraftment was established by bioluminescent imaging (BLI), the mice were treated with a single dose of mock-transduced, CD93-28z or CD93-BBz CAR T cells, then monitored by weekly BLI as a surrogate measurement of AML burden (FIG. 3A). Leukemic burden of mice treated with either CD93-28z or CD93-BBz CAR T cells decreased within one week of treatment compared to mice treated with mock T cells, a difference that persisted for the duration of the experiment (FIGS. 3B-3D). Increased leukemic clearance was observed in CD93-28z CAR treated mice, correlated with greater concentrations of CD4+ and CD8+ T cells isolated from peripheral blood at day 14 post CAR T cell treatment, supporting the importance of in vivo T cell expansion for leukemic clearance (FIG. 3E).

To expand studies to primary human leukemia samples, CD93-CAR T cells were tested in NSG mice sub-lethally irradiated and injected with primary AML cells (FIG. 3F), which expressed uniformly high levels of CD93 (FIG. 3G). The mice were monitored for leukemic engraftment by serial bone marrow aspiration (BMA) and treated with mock-transduced or CD93-CAR-transduced T cells once human leukemia cells comprised on average at least 10% of the cells isolated from the BM. Of note, engraftment levels at time of treatment were variable and some mice had near complete BM invasion by human AML. Regardless of level of engraftment, all mice treated with CD93-CAR T cells experienced full remission measured by flow cytometry from the BM by four weeks and the majority of the mice had no leukemia detectable through 13 weeks post-CAR treatment (FIG. 311). All mice receiving mock transduced T cells succumbed to disease (FIG. 3I). There was no significant difference among the groups in T cell persistence at any time point, and all but one CD93-CAR treated mouse had T cell persistence through 13 weeks (FIG. 3J). BMA at one week post-CAR T cell injection revealed massive cell death in the CD93-CAR treated groups, precluding analysis of leukemic burden at early time points in these groups (FIG. 3K). The mice had minimal systemic toxicity during this time and recovered quickly. A complete blood count two weeks post-CAR T cell treatment demonstrated normal hematologic parameters in the CD93-CAR treated groups compared to leukocytosis, thrombocytopenia, and anemia in the mock-treated group (FIG. 3L). One mouse in the CD93-28z-treated group and two in the CD93-BBz-treated group developed detectable leukemia at later time points, and relapses were driven by CD93 positive cells (FIGS. 3M-3N).

### Example 5

### CD93-CAR T Cells do not Disrupt Hematopoietic Progenitor Viability or Function

This Example describes experiments performed to show CD93-CAR T cells do not disrupt viability or function of non-cancer cells, such as normal hematopoietic progenitors.

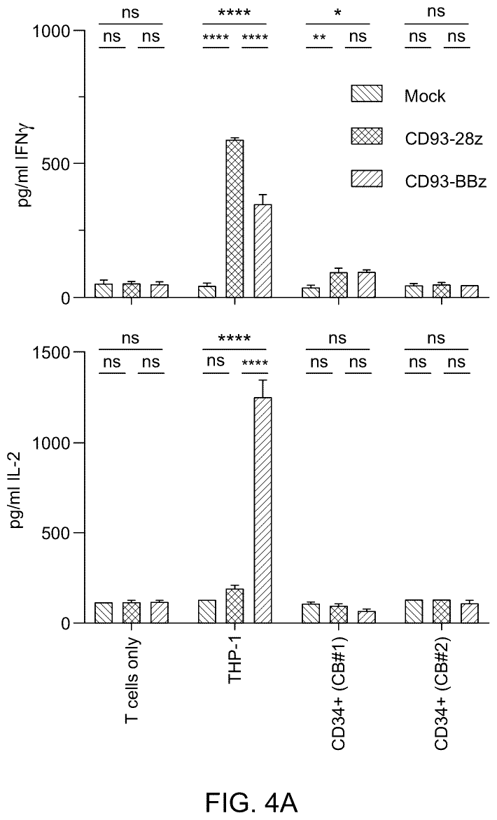

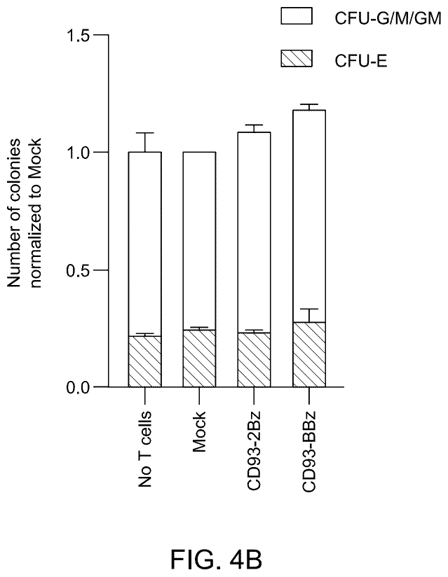

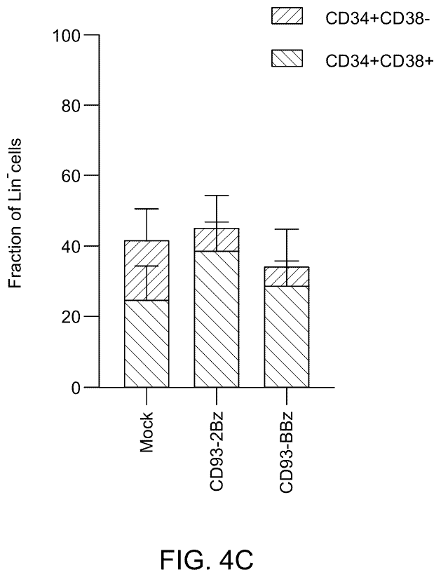

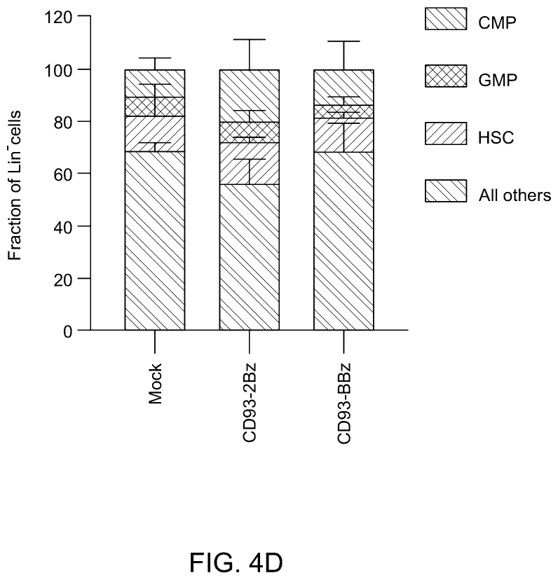

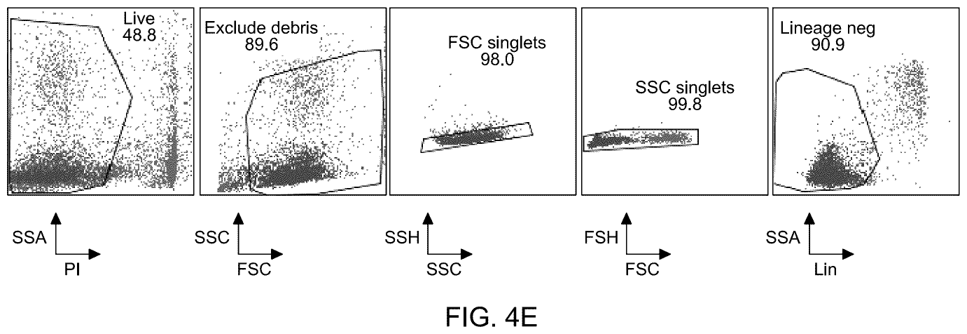

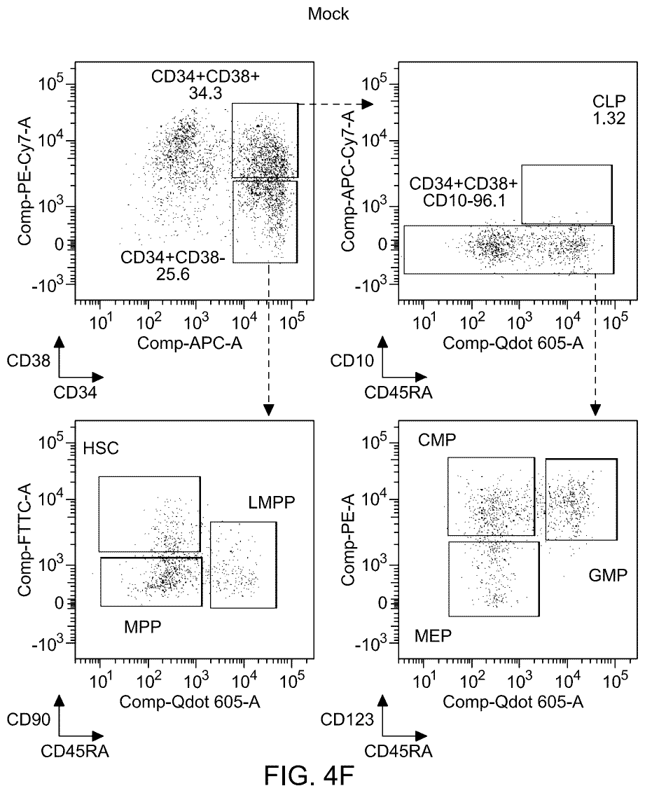

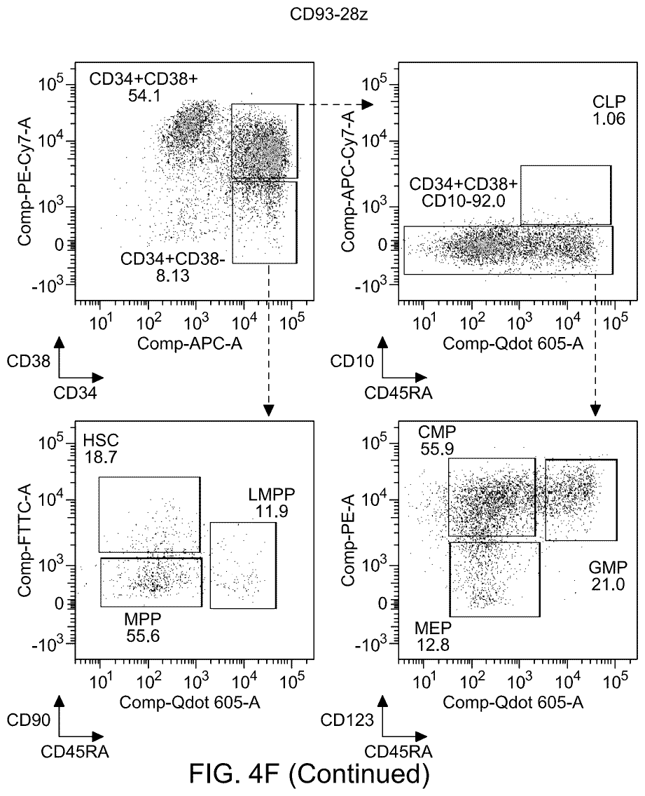

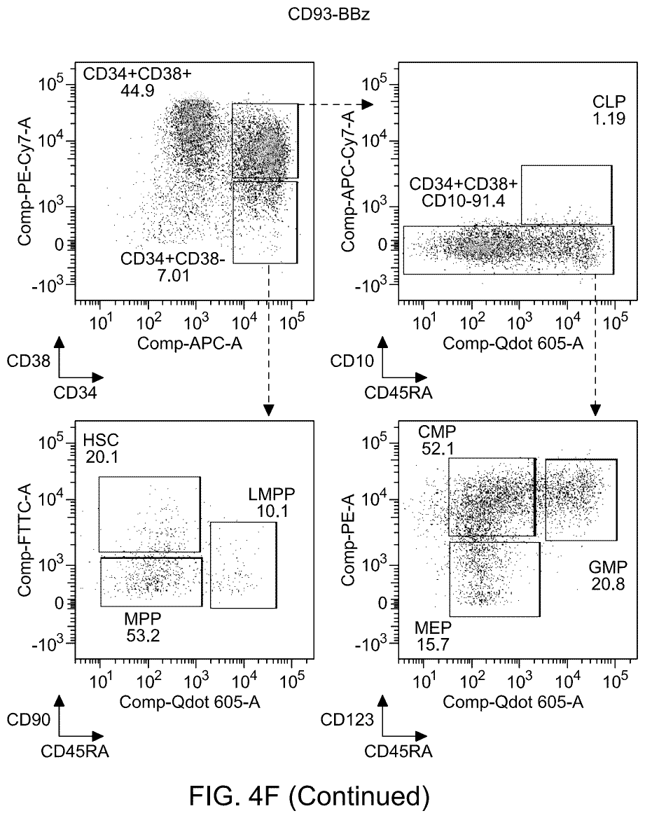

Most AML CAR targets under development are expressed to some extent on normal hematopoietic cells, which is predicted to impact their respective toxicity profiles. Because CD93 expression within the normal hematopoietic compartment is limited to mature myeloid cells (FIGS. 1D-1E), CD93-CAR T cells may have a minimal impact on HSPC viability or function. Indeed, CD93-CAR T cells did not secrete cytokines following exposure to cord blood-derived CD34+ cells, which comprise a variety of HSPC populations (FIG. 4A). To further probe the impact of CD93-CAR T cells on HSPC proliferation and differentiation, cord blood-derived CD34+ cells were untreated or exposed to mock-transduced or CD93-CAR T cells and assayed for colony forming ability. There was no difference among the groups in CFU-E (the bottom shaded block in each bar) or CFU-G/M/GM (the top blank block in each bar) (FIG. 4B). Flow cytometry analysis of HSPC subsets after exposure to CD93-CAR T cells confirmed that the proportion of progenitor cells within each subset, whether segregated by CD34+CD38+/CD34+CD38− cells (FIG. 4C) or more specifically by HSCs, common myeloid progenitors, and granulocyte-macrophage progenitors, was not altered after exposure to CD93-CAR T cells (FIGS. 4D-4F). Together these data support that HSPCs are spared from CD93-CAR T cell toxicity.

### Example 6

CD93 is Expressed on Endothelial Cells with a Similar Expression Pattern to CD123

This Example describes experiments performed to compare expression patterns of CD93 and CD123 on non-cancer normal cells, such as endothelial cells.

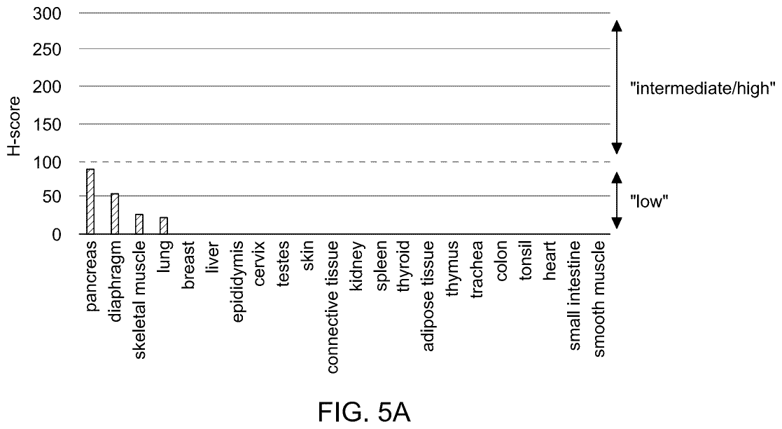

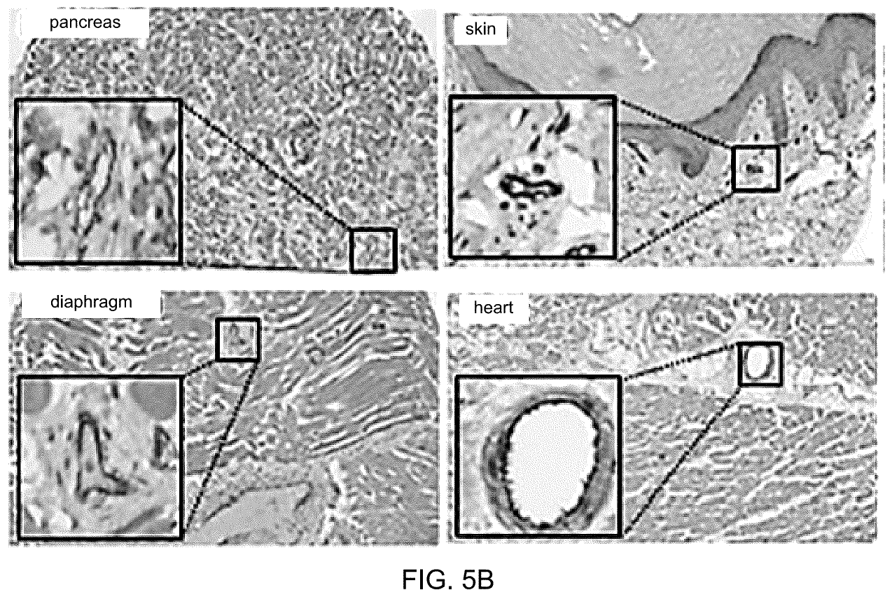

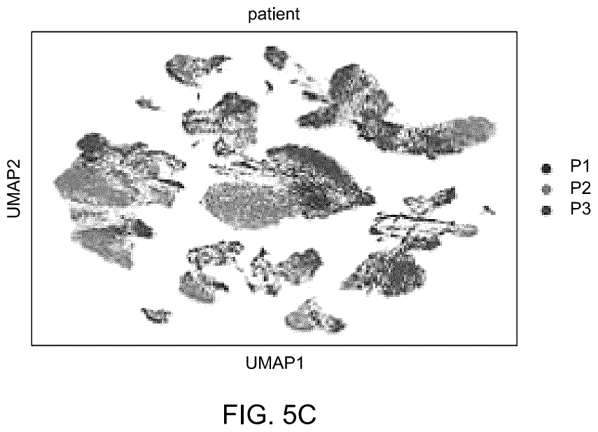

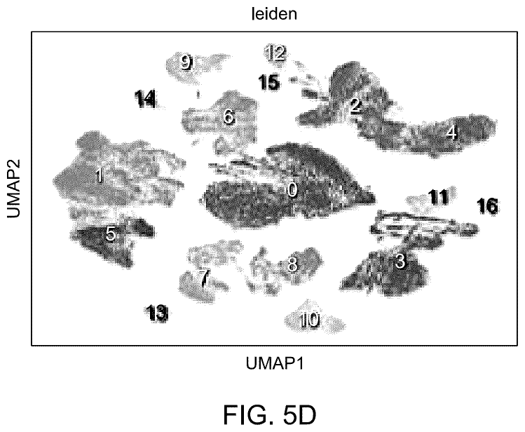

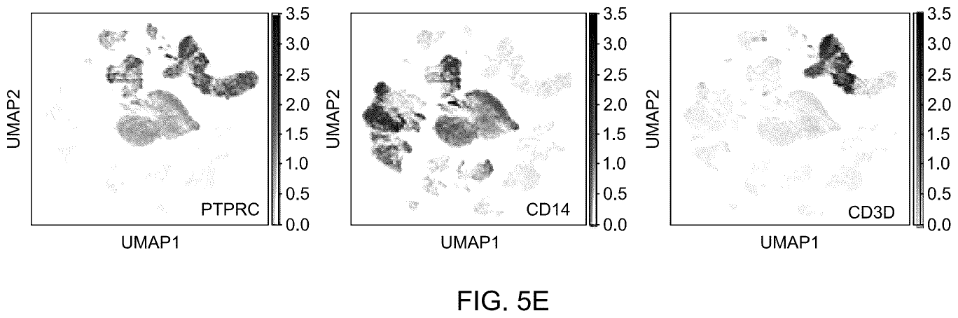

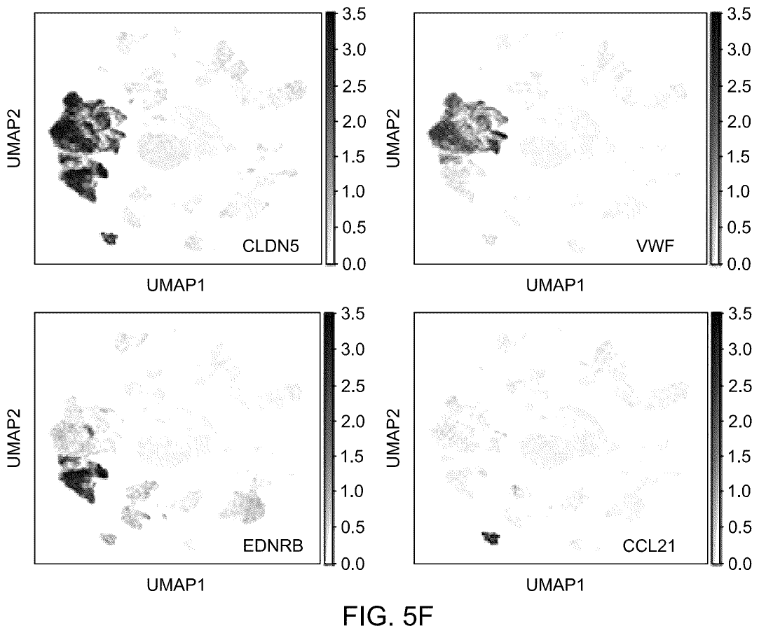

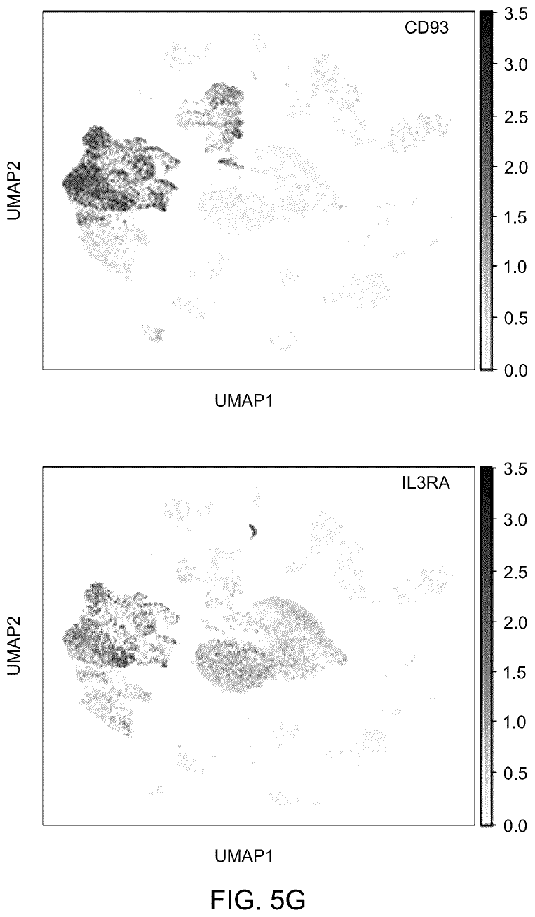

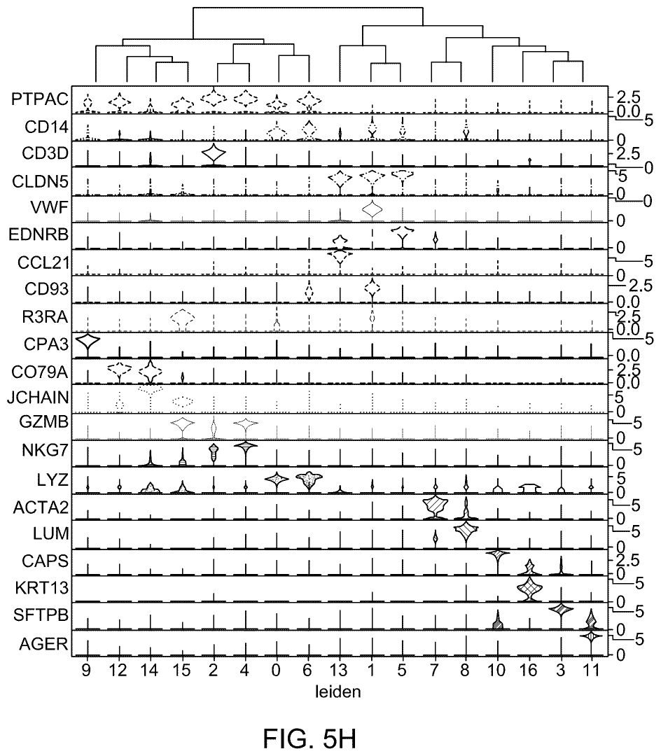

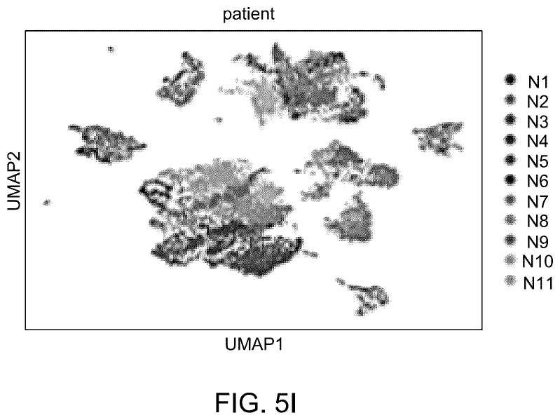

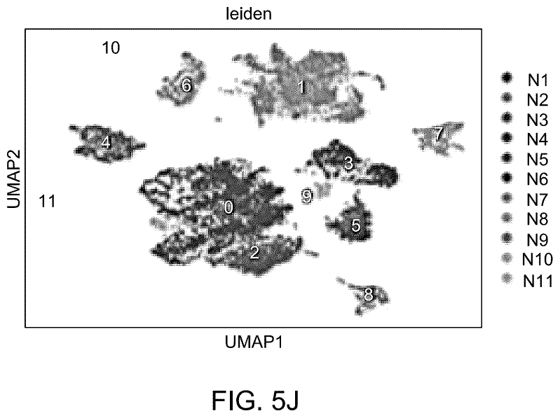

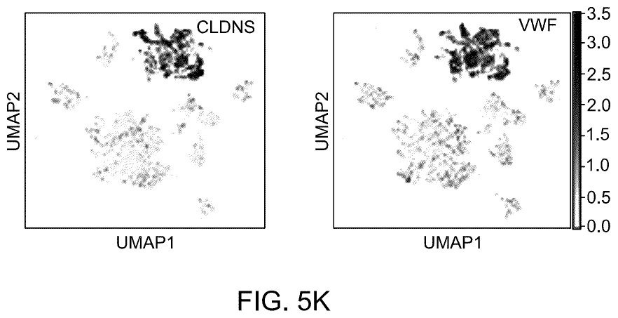

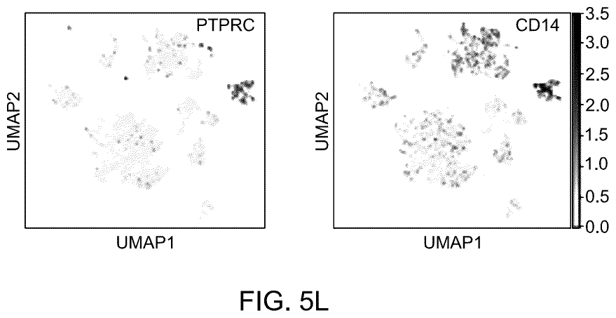

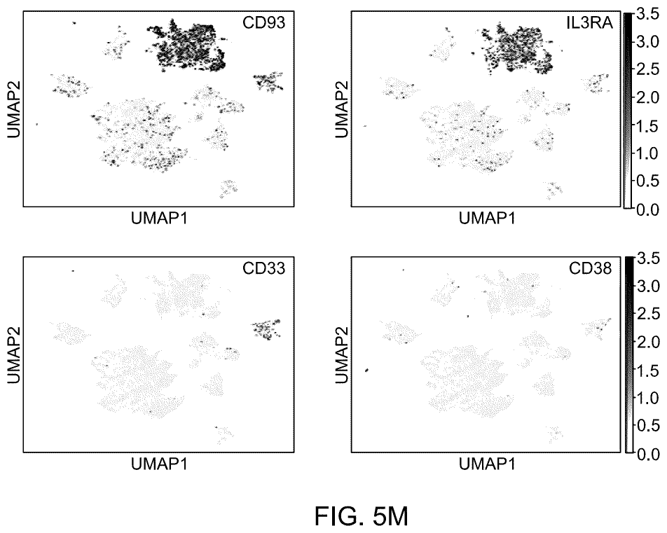

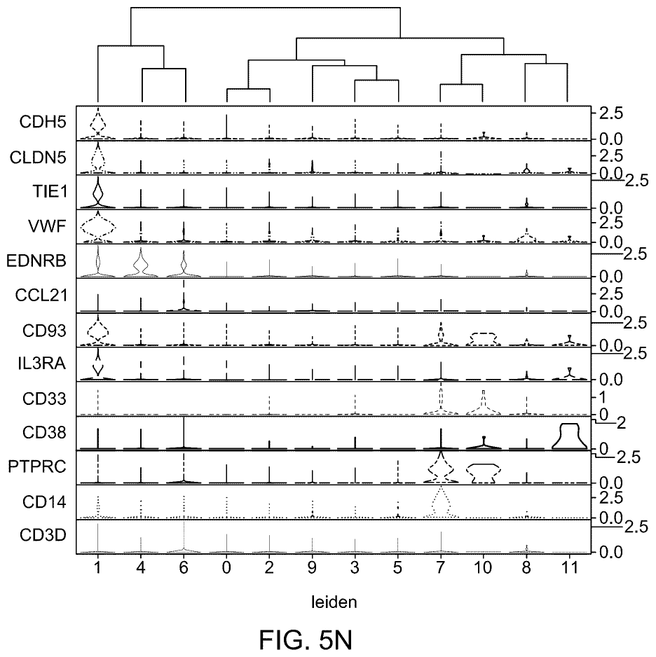

CD93 expression on normal tissue was evaluated by immunohistochemistry of a tissue microarray. H-scores for all tissues analyzed were <100 (FIG. 5A), which is generally accepted as low or no expression (Hirsch et al., Epidermal growth factor receptor in non-small-cell lung carcinomas: correlation between gene copy number and protein expression and impact on prognosis. J Clin Oncol. 2003; 21(20):3798-3807). Despite low overall expression in normal tissues, strong staining was noted in endothelial cells throughout multiple organ systems (FIG. 5B). Analysis of bulk transcriptional data can mask even substantial expression of a target like CD93 on endothelial cells, which comprise only a fraction of cells within any given organ. Endothelial expression and susceptibility to CAR T cell targeting has been considered for other AML targets, most notably CD123 (Cellectis Reports Clinical Hold of UCART123 Studies [press release], 2017; Sun et al., Onco Targets Ther. 2019; 12:4907-4925). Thus, baseline endothelial expressions of CD93 and CD123 were assessed by interrogating a scRNA-seq atlas of healthy lung and paired peripheral blood (FIG. 5C) (Travaglini et al., bioRxiv. 2019:742320). UMAP clustering defined by gene expression profiles resulted in 17 distinct cell populations (FIG. 5D). Hematopoietic cells, defined broadly by PTPRC (CD45), can be found in many clusters, whereas the monocyte marker CD14 and the T lymphocyte marker CD3D have more restricted expression patterns (FIG. 5E). CLDN5, a pan-endothelial marker (Morita et al., Endothelial claudin: claudin-5/TMVCF constitutes tight junction strands in endothelial cells. J Cell Biol. 1999; 147(1):185-194), is expressed highly in clusters 1, 5, and 13, whereas VWF, EDNRB, and CCL21 likely define stalk-like, tip-like, and high endothelial venule endothelial subsets, respectively (FIG. 5F) (Ager and May, Understanding high endothelial venules: Lessons for cancer immunology. Oncoimmunology. 2015; 4(6): e1008791; Chen et al., The endothelial tip-stalk cell selection and shuffling during angiogenesis. J Cell Commun Signal. 2019; 13(3):291-301). CD93 and IL3RA (CD123) are each expressed in a subset of myeloid CD14+ cells as expected but are also clearly expressed in endothelial clusters (FIG. 5G). CD93 and IL3RA expression are compared to the most distinct signature genes for each cell type in a violin plot (FIG. 5H). To ensure the expression patterns are not unique to lung tissue, a similar analysis was performed on scRNA-seq data from non-malignant pancreatic tissue, which confirmed clear expression of CD93 and IL3RA within the endothelial compartment despite minimal expression in other clusters (FIGS. 5I-5N).

### Example 7

On-Target, Off-Tumor Toxicity of CD93 CAR T Cells Against Endothelial Cells is Mitigated with a NOT-Gated CAR T Cell Strategy In Vitro

This Example describes experiments performed to investigate a CAR T cell strategy to mitigate the on-target, off-tumor toxicity of CD93 CAR T cells.

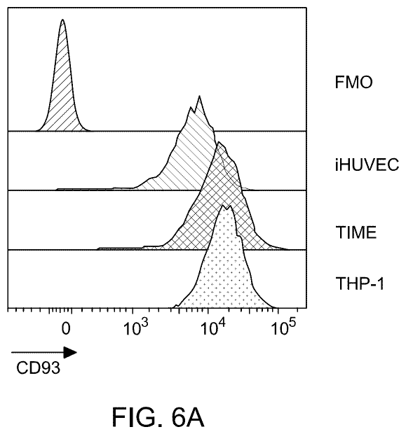

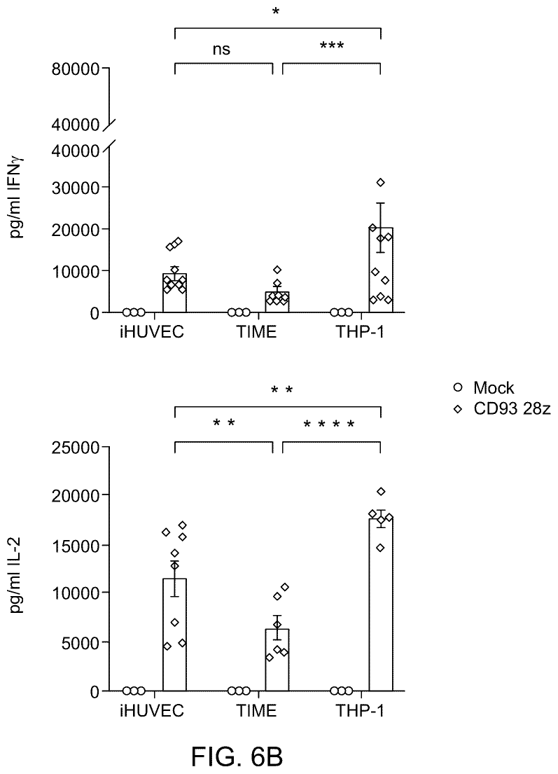

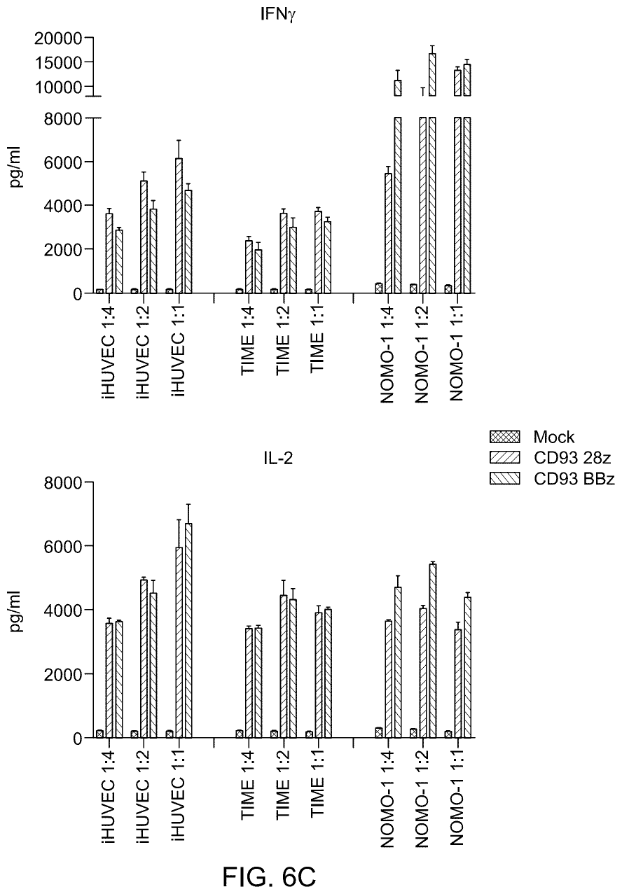

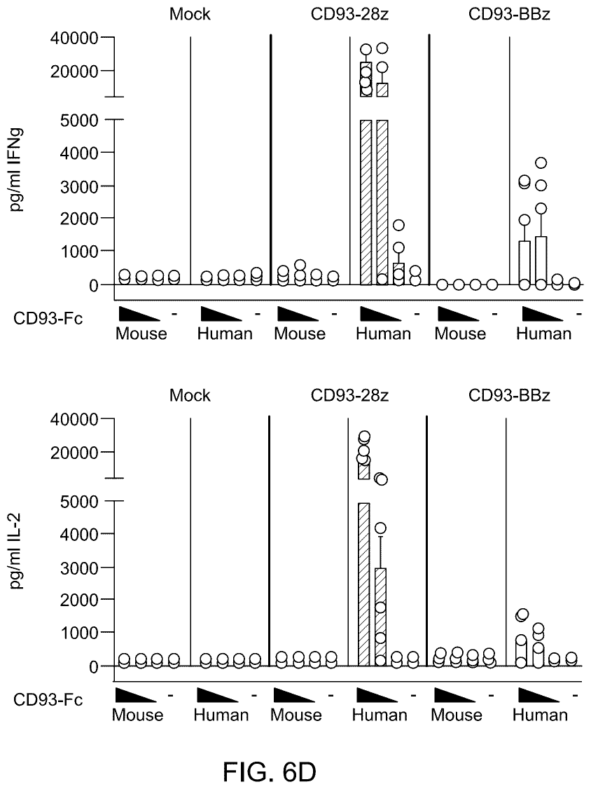

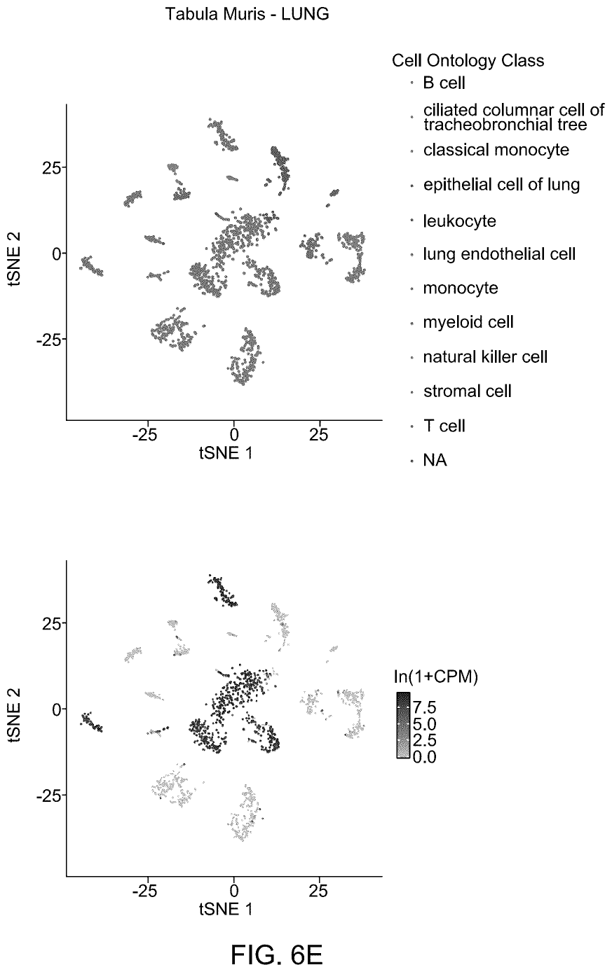

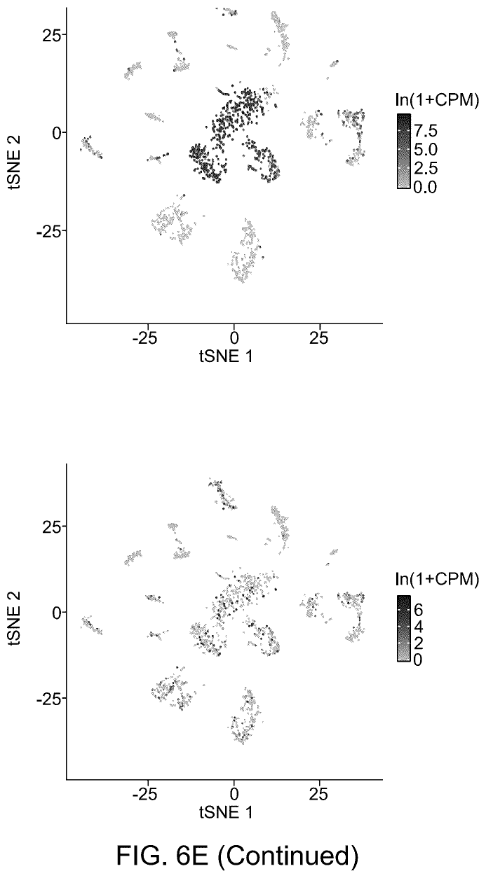

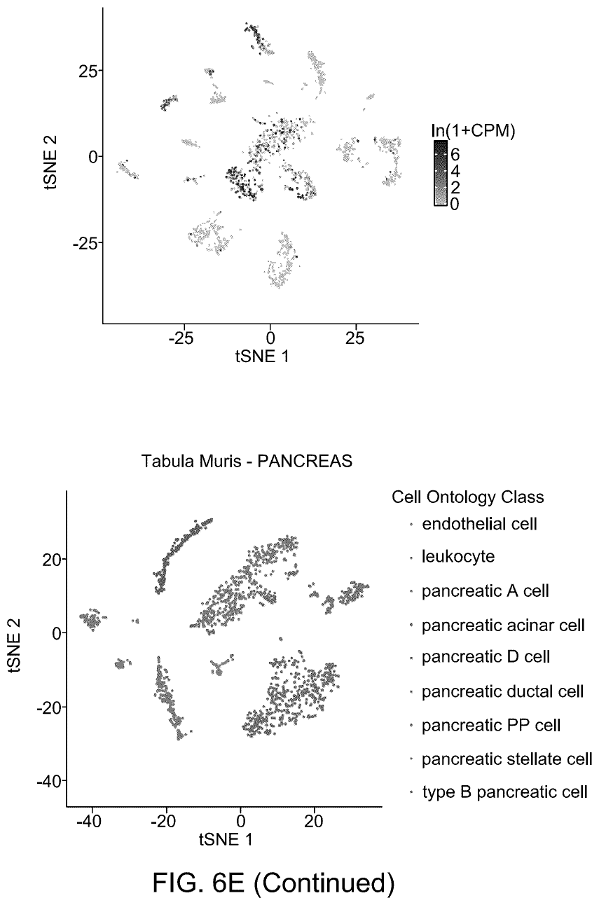

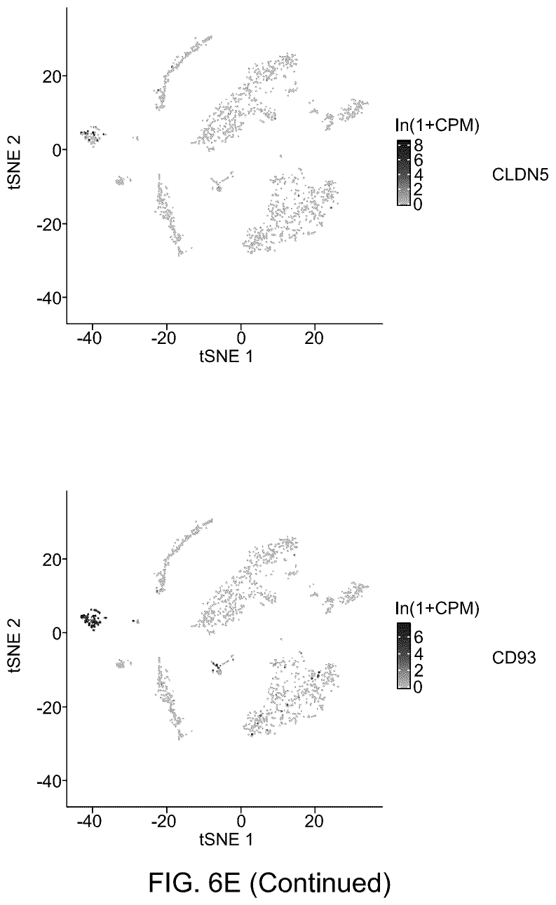

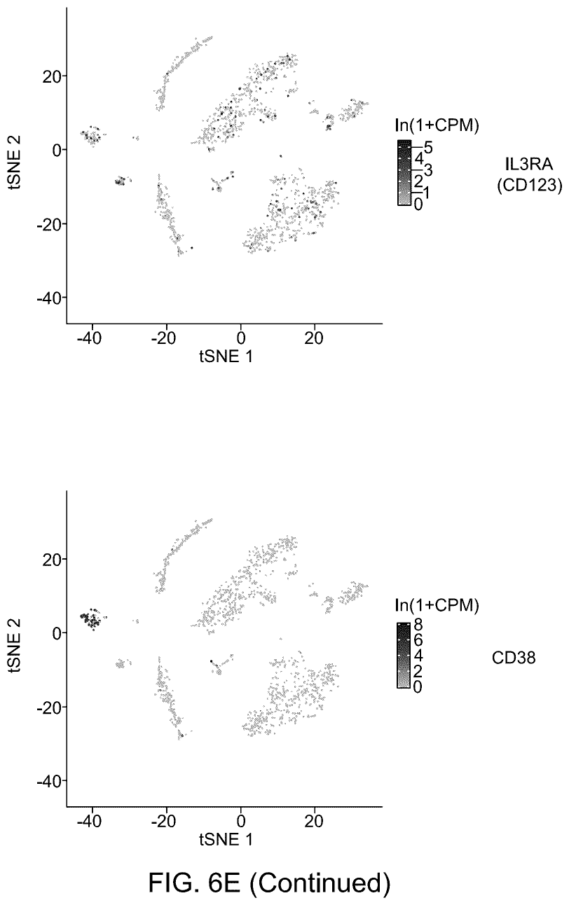

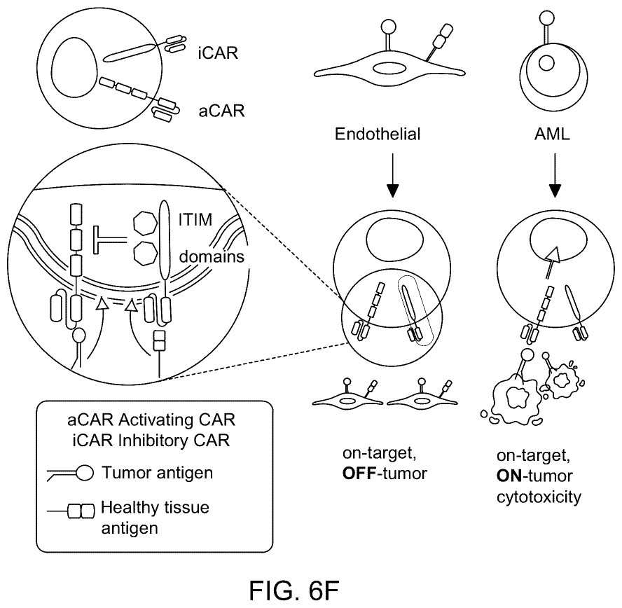

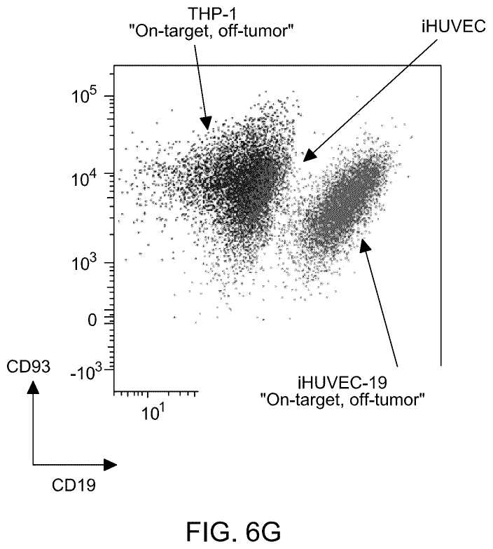

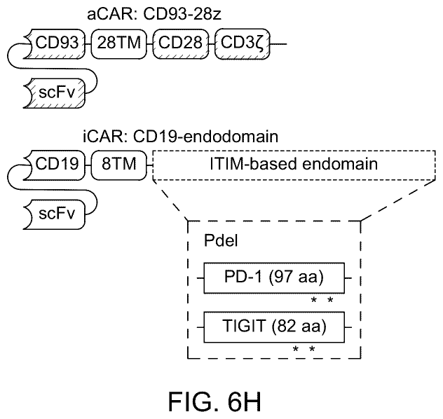

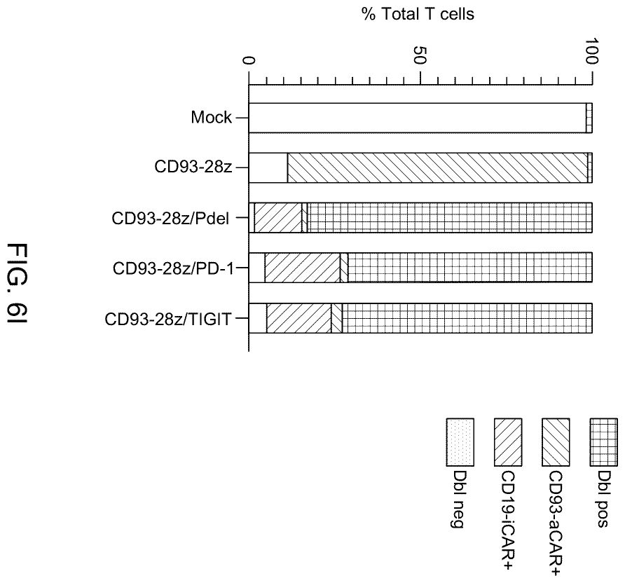

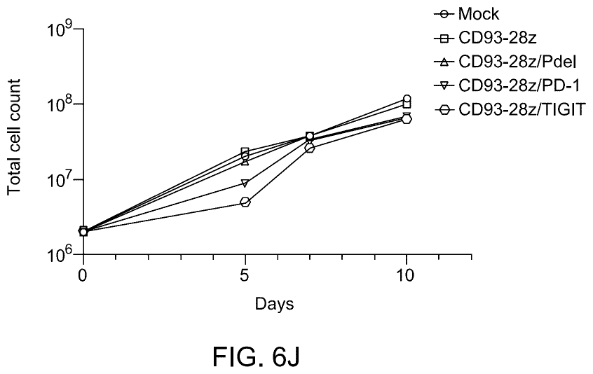

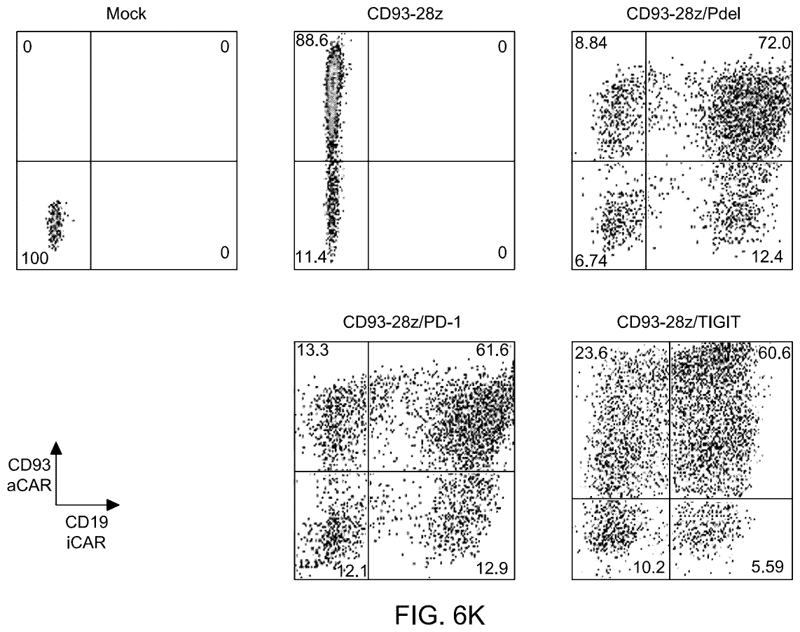

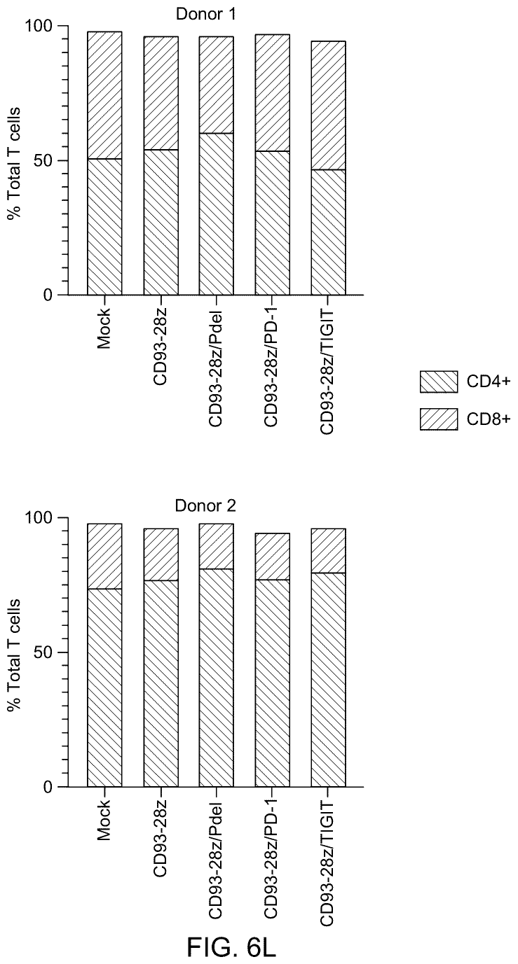

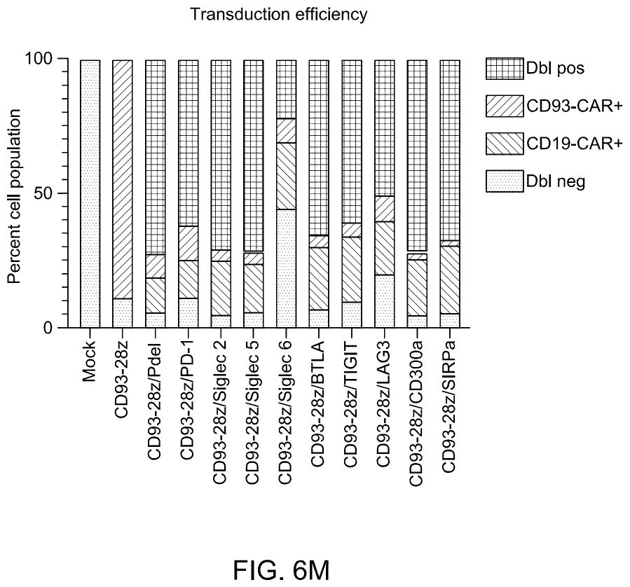

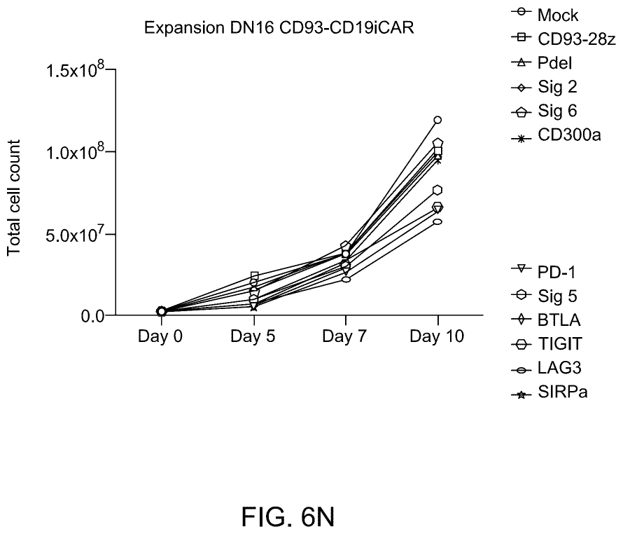

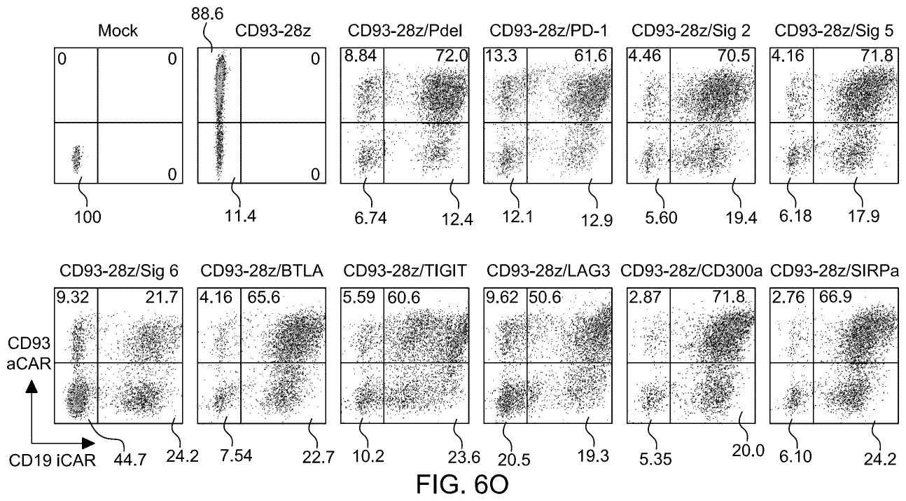

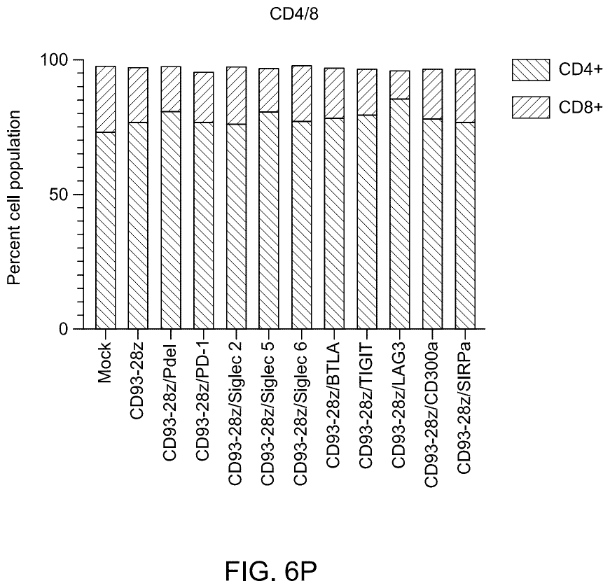

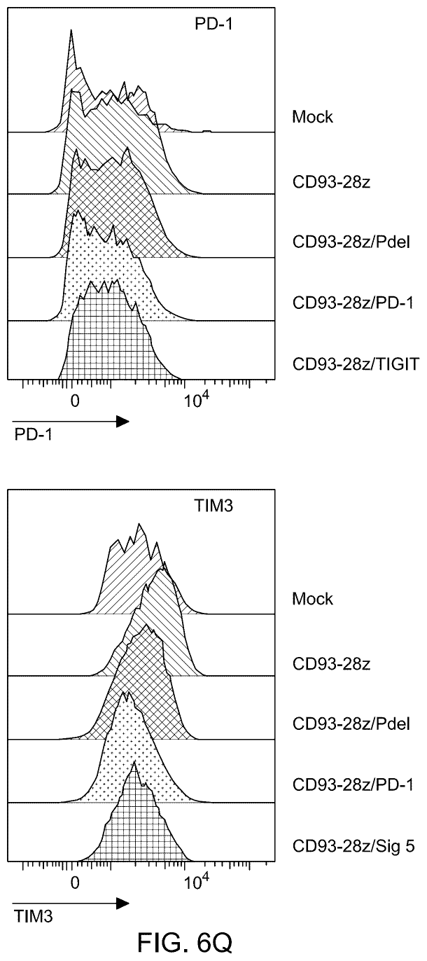

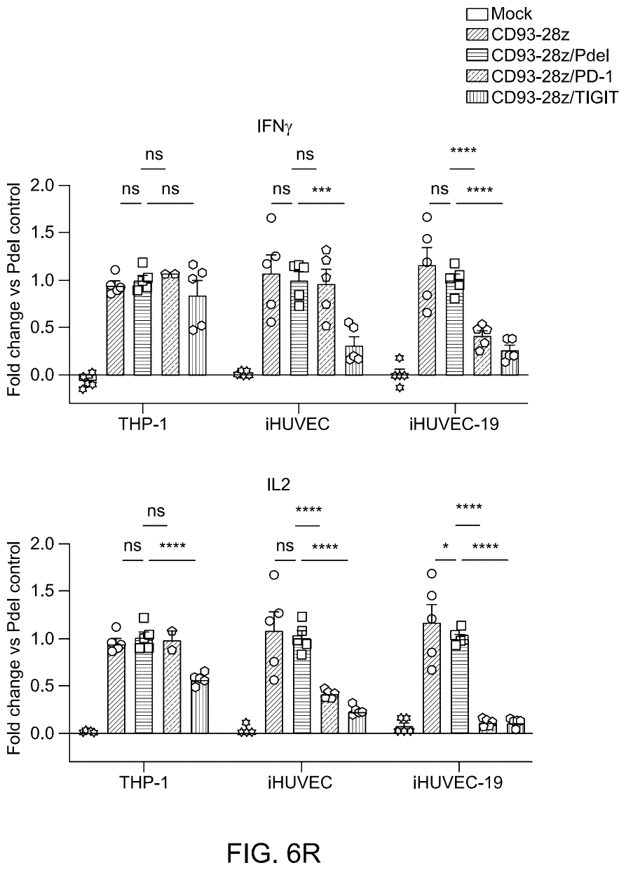

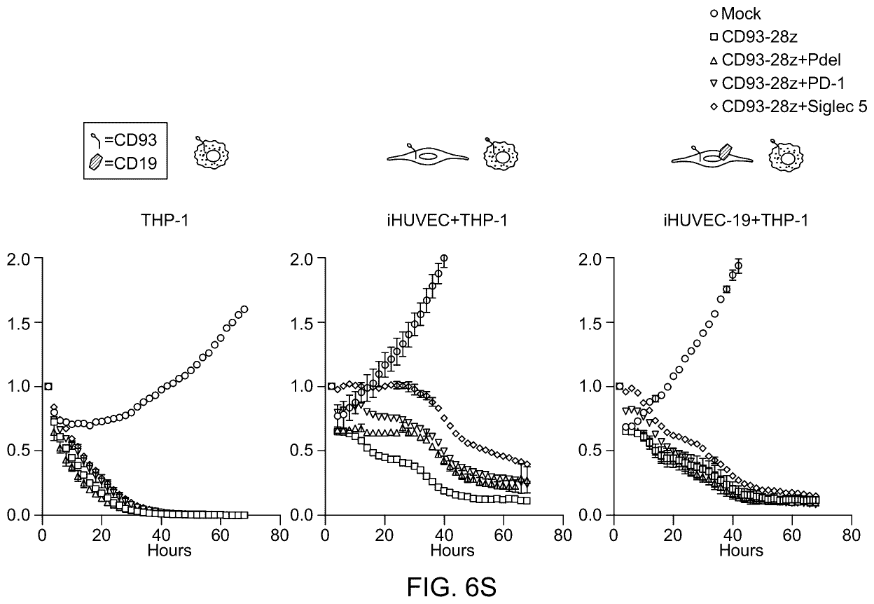

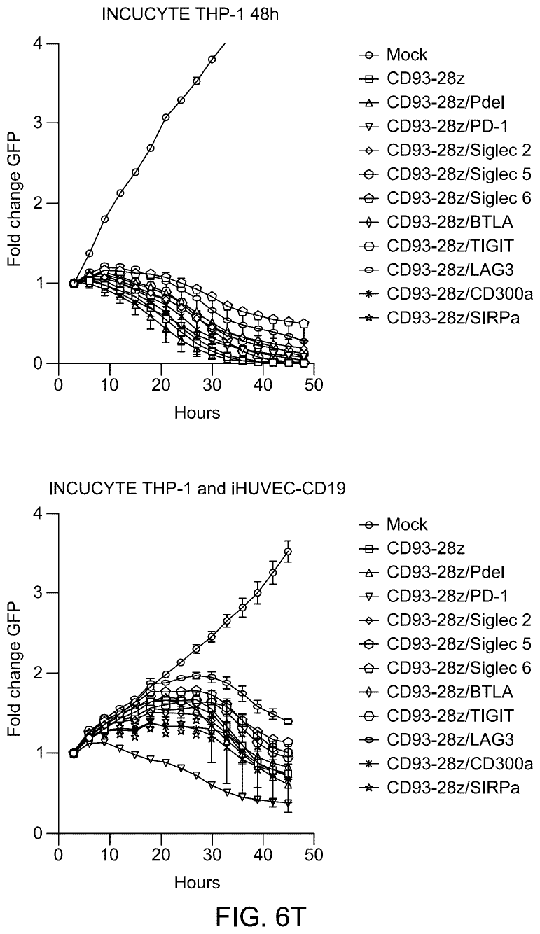

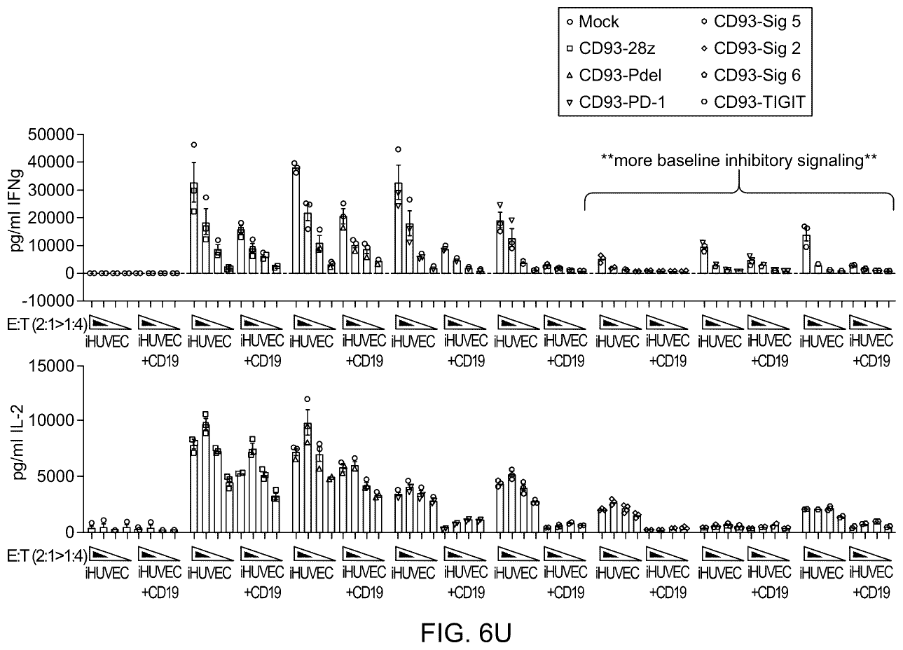

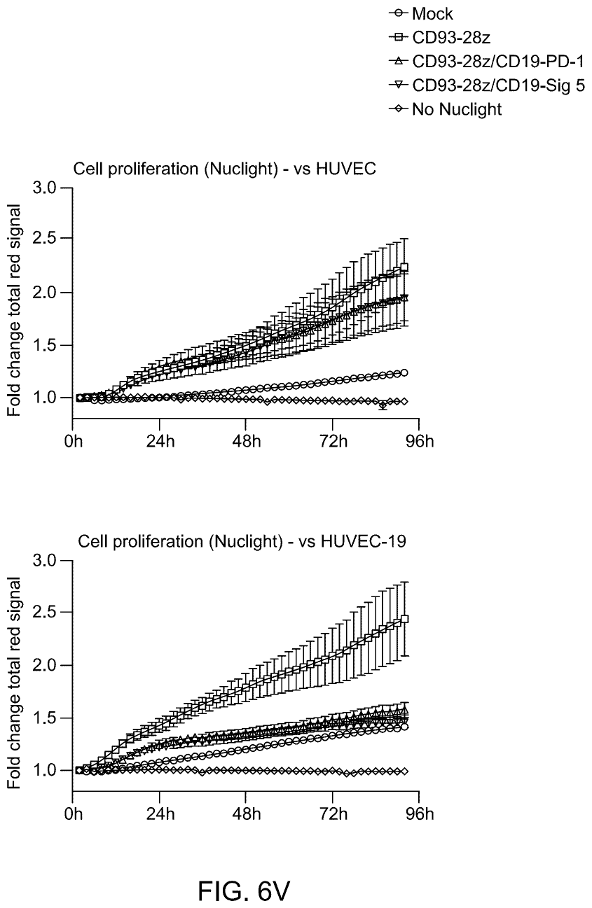

Flow cytometry confirmed that CD93 expression on endothelial iHUVEC and TIME cell lines was nearly as high as on the AML cell line THP-1 (FIG. 6A). Furthermore, CD93-28z CAR T cells secreted cytokines upon exposure to both iHUVEC and TIME endothelial cell lines at different E:T ratios (FIGS. 6B-6C). Although the scFv contained in the CD93-CAR studied here does not cross-react with murine CD93 and thus endothelial toxicity cannot be assessed in murine models (FIG. 6D), single cell transcriptomic analysis of the Tabula Muris database demonstrate a similar expression pattern of CD93 on murine endothelial cells (FIG. 6E).

It has been demonstrated that on-target, off-tumor toxicity can be overcome with NOT-gated CAR T cells that express a second inhibitory CAR (iCAR) specific for an antigen present on healthy cells but absent on tumor cells. Given the propensity for myeloid and endothelial co-expression of target antigens, a NOT-gate strategy targeting molecules expressed on endothelium but not myeloid cells may overcome this toxicity. To test this, an in vitro model of “on-target, off-tumor” endothelial cells was created to assess the specificity of iCAR-based NOT-gated CAR T cells (FIG. 6F), wherein the iHUVEC endothelial cell line was engineered to stably express a truncated CD19 (extracellular and transmembrane domain only) (FIG. 6G). CD19-specific iCARs were designed to include signaling endodomains from immunoreceptor tyrosine-based inhibitory motifs (ITIM)-containing proteins including PD-1 and TIGIT, or no intracellular signaling domain (Pdel) as a control (FIG. 611). The NOT-gated CD93-CAR T cell products exhibited robust expansion, high transduction efficiency, and CD4/8 ratios influenced by donor, not by the presence of the iCAR construct (FIGS. 6I-6L). The similar results were also observed in T cells expressing CD93-CAR molecules containing other ITIM-containing proteins, such as Sig 2, Sig 6, CD300a, Sig 5, BTLA, LAG3 and SIRPa (FIGS. 6M-6P). Cells that co-expressed iCARs having ITIM-containing endodomains demonstrated decreased expression of exhaustion markers PD-1 and TIM3 at baseline compared to CD93-28z (FIG. 6Q). Upon co-culture with iHUVEC-19 “on-target, off-tumor” cells, NOT-gated CD93-CAR T cells secreted significantly less IFNγ and IL-2 compared to CD93-28z/Pdel control. IFNγ production against the “on-target, on-tumor” AML cell line THP-1 was preserved across all constructs. Similar to previous reports (e.g., Fedorov et al., PD-1- and CTLA-4-based inhibitory chimeric antigen receptors (iCARs) divert off-target immunotherapy responses. Sci Transl Med. 2013; 5(215):215ra172), the PD-1-based iCAR provided antigen-specific inhibition of cytokine production. The novel TIGIT-based iCAR inhibited cytokine production equally as well in endothelial cells, upon recognition of both iHUVEC and iHUVEC-19, suggesting more baseline antigen-independent inhibitory signaling compared to the PD-1-based iCAR (FIG. 6R). The cytotoxicity of CD93 CAR-T cells against THP-1 cells expressing CD93 was preserved in presence of various CD19-specific iCARs (FIGS. 6S-6T).

As shown in FIG. 6C, CD93 CAR-T cells secreted cytokines upon exposure to both iHUVEC and TIME endothelial cell lines at different E:T ratios. A similar experiment was performed using T cells expressing various CD93 CAR molecules. As shown in FIG. 6U, under effector to target (E:T) ratios between 1:4 and 2:1, all tested T cells expressing the CD93 CAR (aCAR) and the CD19 CAR (iCAR) molecules produced less cytokines upon exposure to the iHUVEC-CD19 endothelial cell lines, compared to iHUVEC cells, illustrating the mitigation of the on-target, off-tumor toxicity of CD93 CAR T cells by the NOT-gate system.

T cells transduced with mock, CD93 aCAR only, or CD93 aCAR/CD19 iCAR were labeled with Nuclight red to monitor for T cell proliferation, then were co-cultured with HUVEC or HUVEC-19 target cells. T cell proliferation was measured over 96 h by Incucyte assay. As shown in FIG. 6V, proliferation, after co-culturing the T cells with target HUVEC (the top panel) or HUVEC-19 (the bottom panel) cells, was increased for T cells expressing the CD93 aCAR construct alone (i.e., CD93-28z). Such increase of cell proliferation was reduced when co-expressing CD93 aCAR and CD19 iCAR constructs in T cells.

### Example 8

### Defining Endothelial- and AML-Predominant Expression at Baseline and in an Inflammatory Microenvironment can Inform Rational Combinatorial CAR T Cell Strategy

This Example describes experiments performed to identify surface protein expression levels on endothelial or AML cells with or without cytokines.

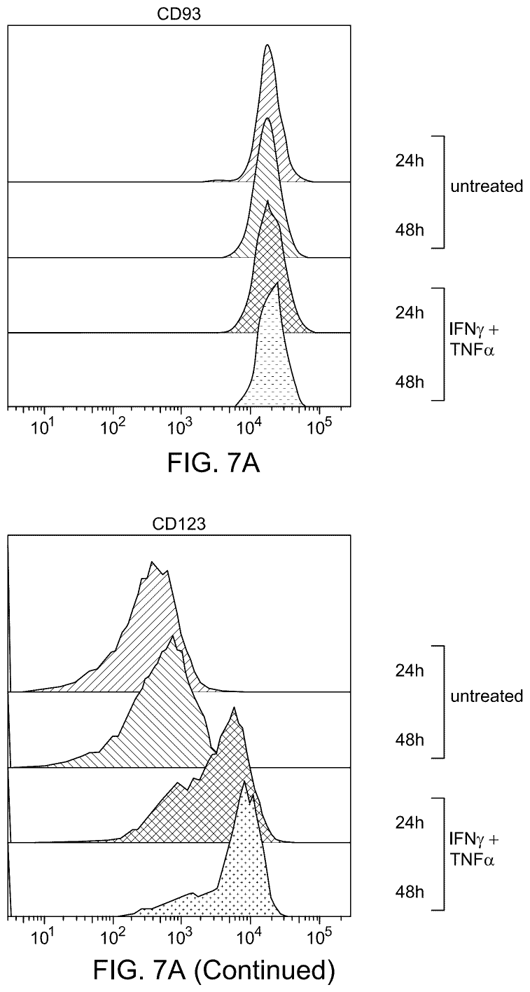

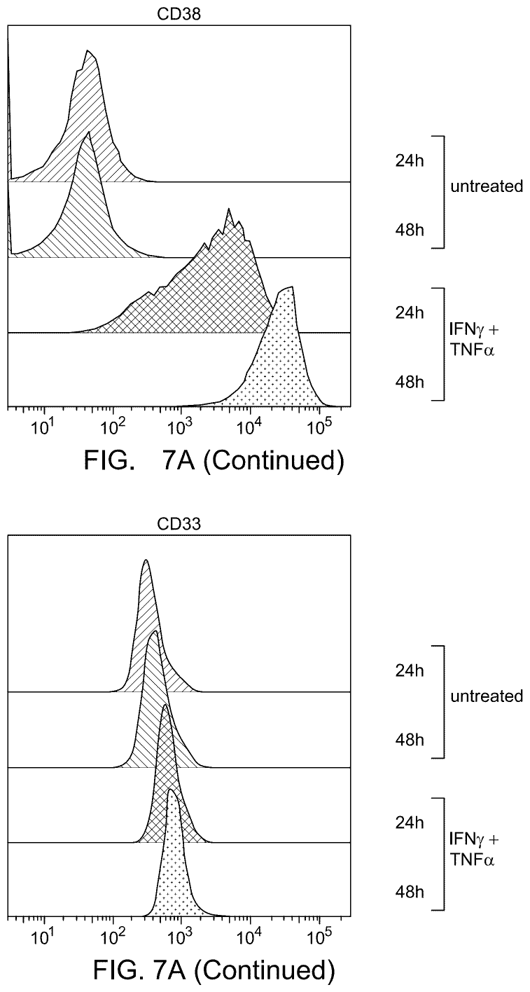

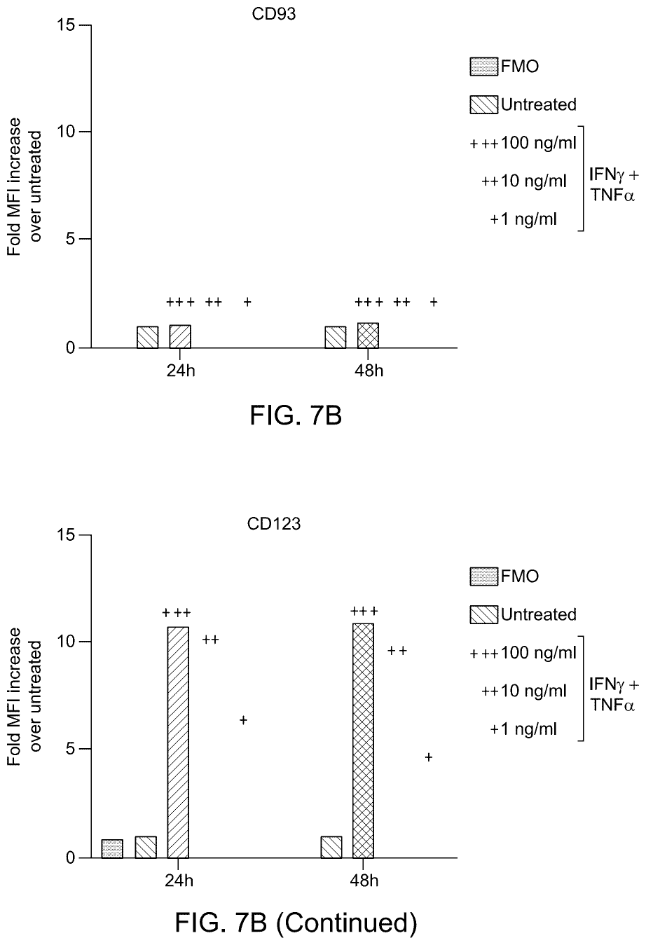

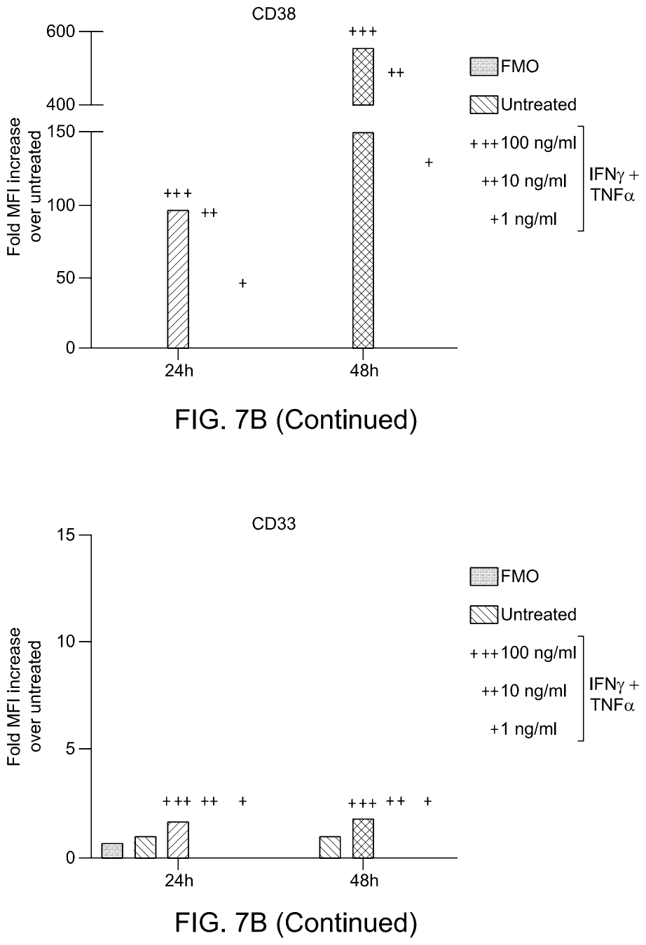

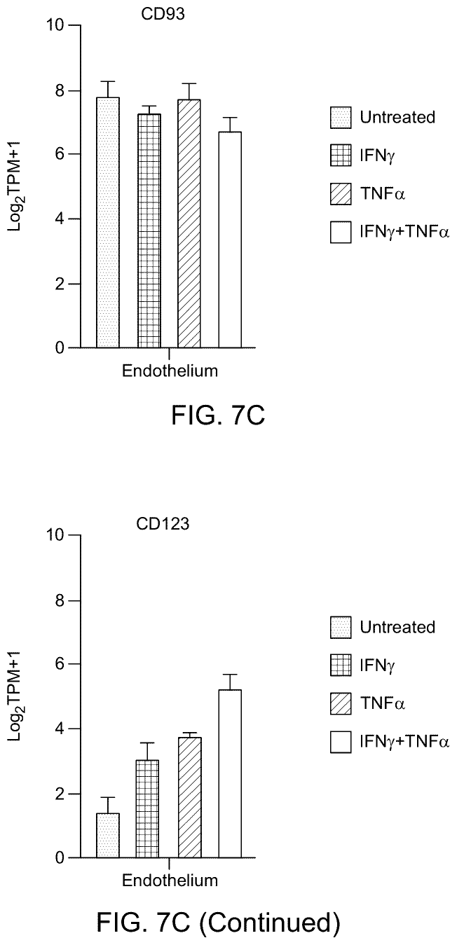

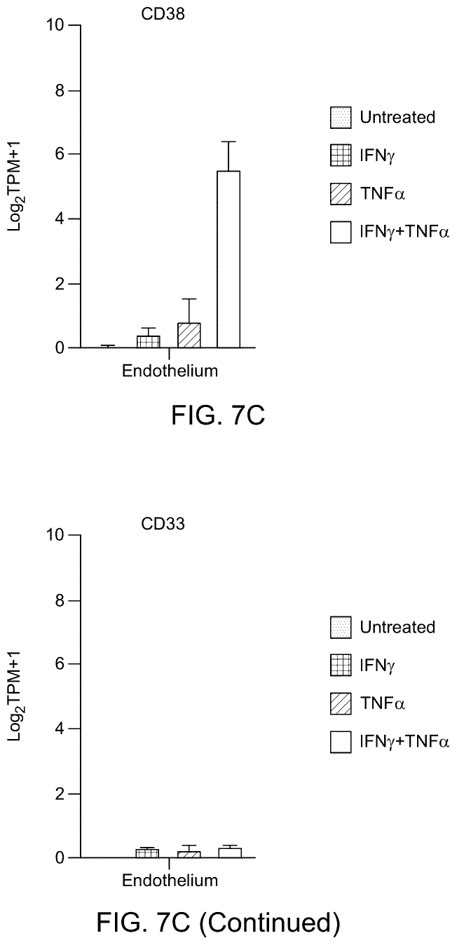

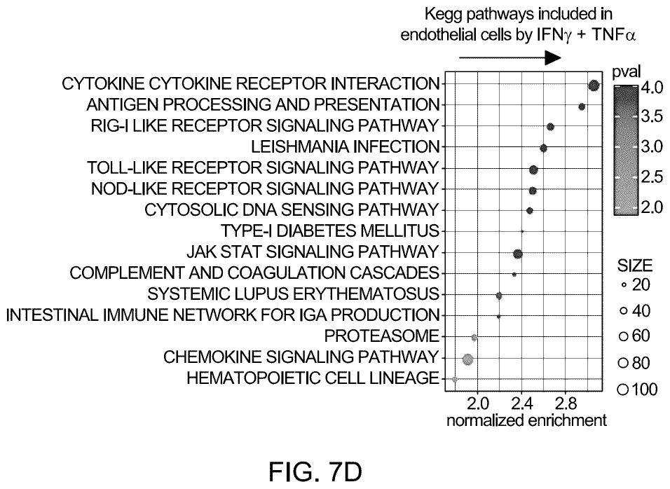

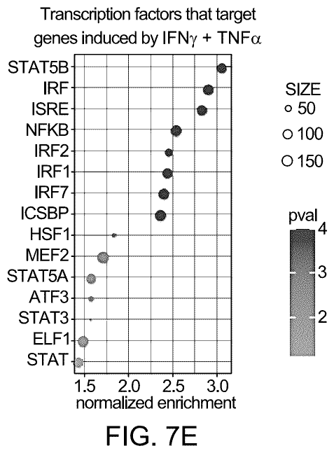

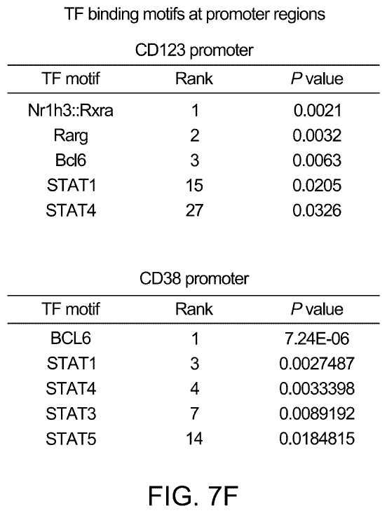

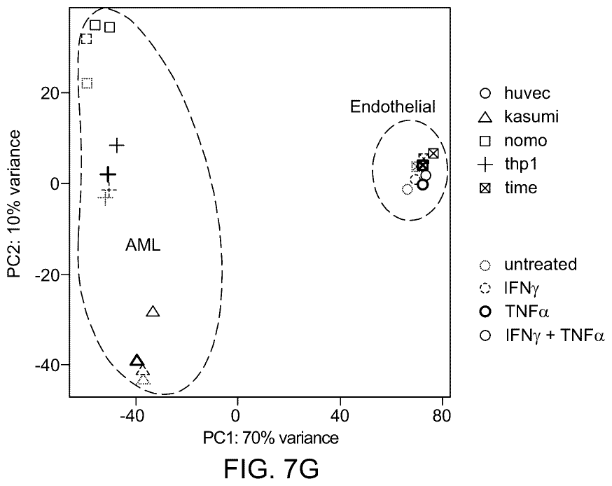

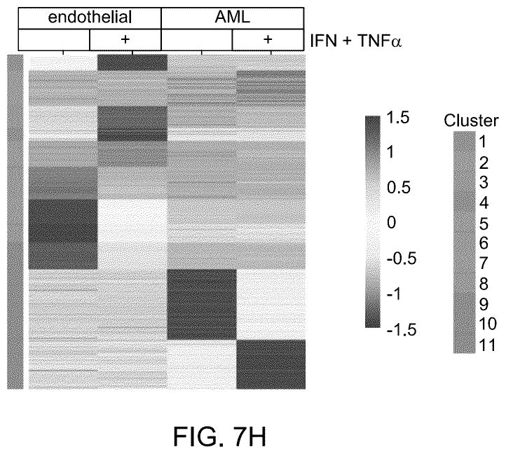

Previous work has demonstrated cytokine inducibility of CD123 expression on endothelial cells that may increase susceptibility to CAR T cell toxicity (Sun et al., Onco Targets Ther. 2019; 12:4907-4925). However, dynamic expressions have not been described for other AML targets. Therefore, a targeted expression analysis was performed for CD93 and other selected AML targets on endothelial cell lines in the absence or presence of pro-inflammatory cytokines IFNγ and TNFα. Cell surface phenotypic analysis revealed that CD123 and CD38 were not expressed highly at baseline on the TIME endothelial cell line, but increased substantially upon exposure to IFNγ and TNFα (FIG. 7A). Cytokine-driven inducible expression was both time- and dose-dependent (FIGS. 7A-7B). In contrast, CD33 was not expressed on endothelial cells under any condition. Targeted RNA transcriptional analysis reinforced these expression patterns (FIG. 7C). Pathway analysis suggests that the changes seen in CD123 and CD38 expression are linked by enhancement of the JAK-STAT signaling pathway (FIGS. 7D-7F).

FIGS. 6A-6V provide proof-of-concept that NOT-gated CAR T cells endowed with an iCAR specific for an endothelial cell-specific antigen could overcome on-target, off-tumor toxicity of an AML CAR directed against a target also expressed on endothelial cells. However, the optimal endothelial-specific iCAR target has not been identified. To advance this objective, an unbiased transcriptional analysis was implemented for differential gene expressions between endothelial and AML cells, both at baseline and in a pro-inflammatory microenvironment. To ensure consistency and to avoid a cell-line specific phenomenon, bulk RNA-seq was performed on two endothelial cell lines and three AML cell lines that were either untreated or incubated with cytokines for 24 h. Principal component analysis (PCA) revealed very little transcriptional variance between the two endothelial cell lines, which formed a grouping distinct from the AML cells. Each AML cell line under all treatment conditions clustered as a group divergent from the others, as would be predicted from their inherent genetic variability (FIG. 7G). When filtering by cell surface designation, genes can be grouped into 11 clusters that emphasize differences between endothelial cells and AML cells, in untreated or in cytokine-treated conditions (FIG. 711). Based on initial filtering parameters, 232 candidate targets were identified for an iCAR-based NOT-gate, which include the well-recognized endothelial cell surface molecules PECAM1 and TIE1 (FIG. 7I). More work will be necessary to validate these as candidate iCAR targets, including by verifying preferential endothelial cell surface protein expression compared to AML, confirming lack of expression across a variety of other normal tissues, and identifying available scFv sequences from which to generate iCARs. Ultimately, this strategy could result in an effective and specific NOT-gate platform that could allow for translation of CAR T cells with strong reactivity toward AML expressed antigens such as CD93 and CD123 while avoiding endothelial cross-reactivity.

### Example 9

### Further Developing of NOT-Gated CAR-T Systems

This Example describes experiments performed to test functions of NOT-gated CAR-T systems with different elements, such as iCARs having various hinge/TM domains and aCARs targeting various antigens.

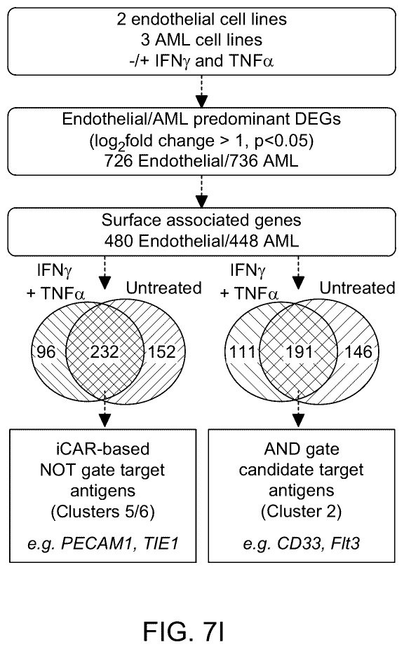

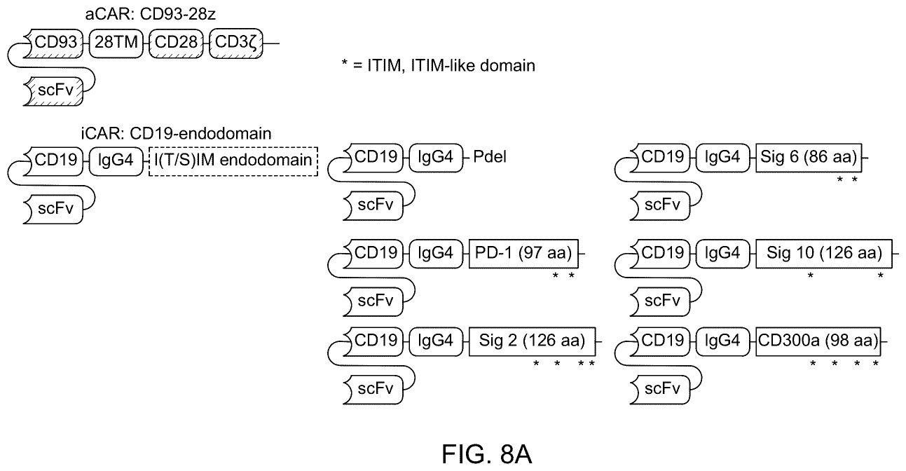

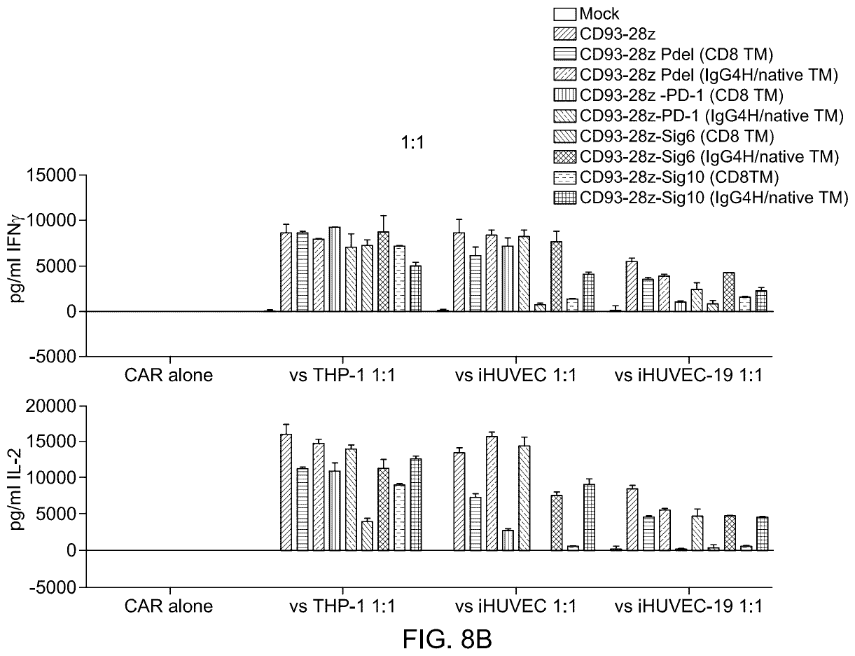

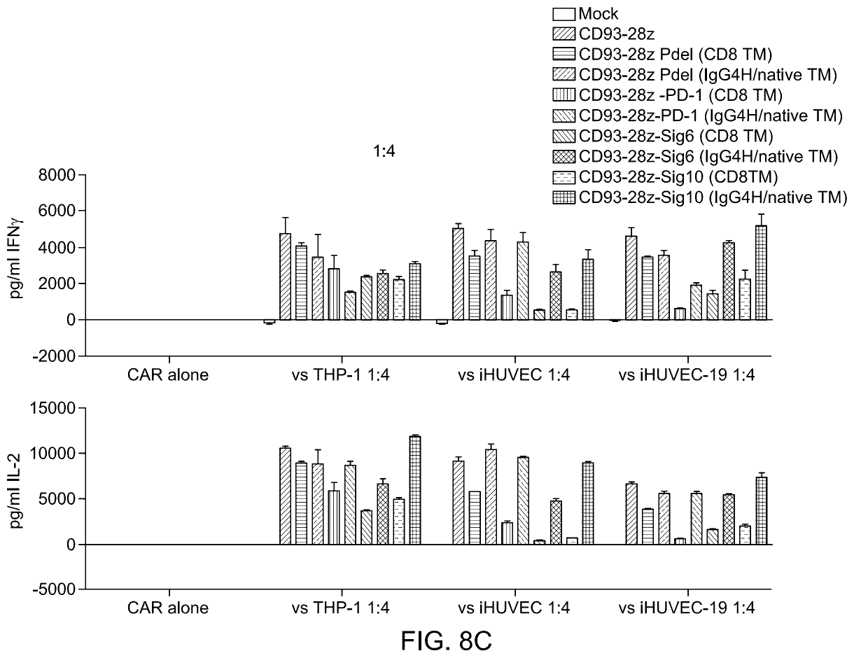

The iCAR molecule described herein may optionally contain a hinge domain. Various exemplary CD19-specific iCAR constructs were prepared, containing either a CD8 TM domain or an IgG4 hinge domain and a native TM domain, plus various internal signaling domains (FIG. 8A). The cytokine production levels of NOT-gated CAR-T cells expressing both the CD93 CAR (aCAR), as described above, and any one of the iCARs against THP-1 cells, iHUVEC cells, or iHUVEC-CD19 cells were measured and compared under an E:T ratio of 1:1 (FIG. 8B) or 1:4 (FIG. 8C). As the result, iCAR constructs have various capacities to reduce cytokine production (FIGS. 8B-8C).

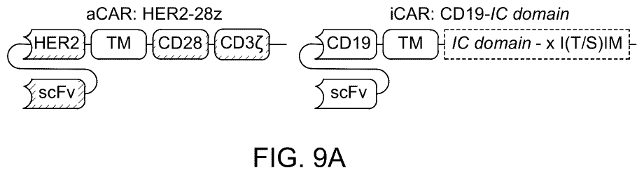

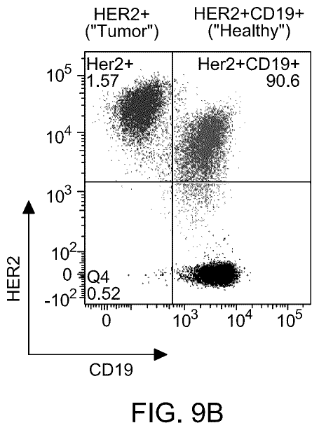

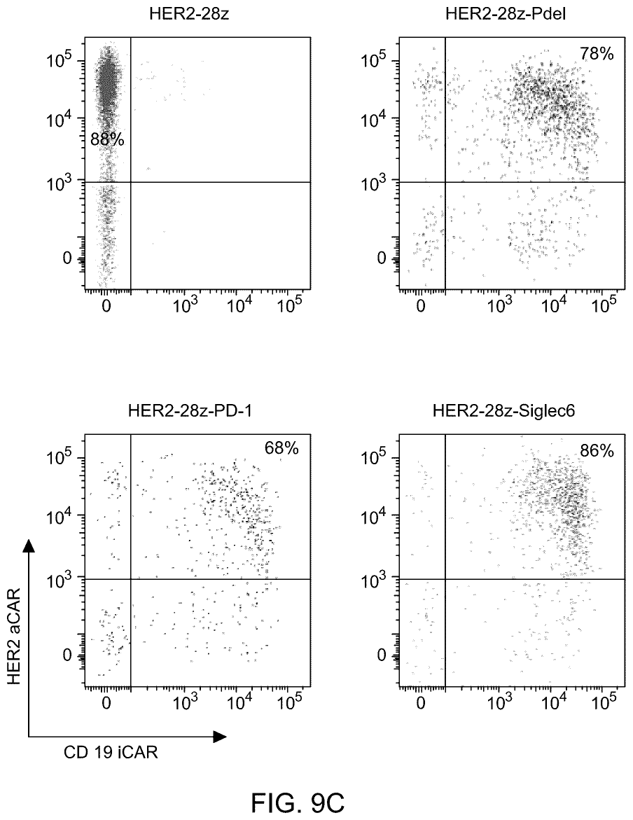

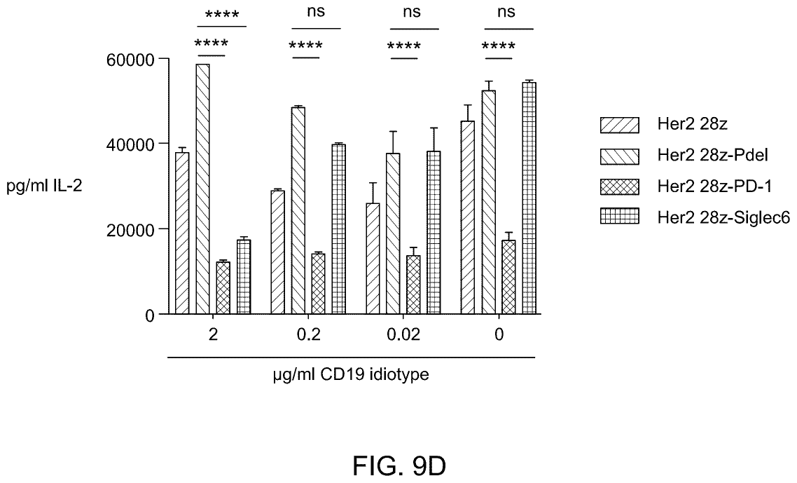

The aCAR molecule described herein may recognize other antigens. For example, instead of CD93, an aCAR specifically binding to HER2 was constructed to combine with iCAR constructs to form NOT-gated CAR-T systems (FIG. 9A). As the result, both NOT-gated CAR-T systems containing PD-1 or Siglec 6 in the iCAR molecule had reduced cytokine production upon exposure to different concentrations of anti-CD19 idiotype antibodies (FIG. 9D).

### Example 10

### NOT-Gated CAR-T Systems Containing CD93 CAR T Cells

This Example summarizes the results of exemplary experiments described herein about the NOT-gated CAR-T systems containing CD93 CAR-T cells, including their capability to eliminate cancer cells (e.g., AML cells) and reduce the on-target, off-tumor toxicity to non-cancer cells.

Outcomes remain dismal for patients with relapsed or refractory AML despite the emergence of many targeted therapies over the last decade. Enthusiasm for adding CAR T cells and other immunotherapies to the AML treatment arsenal is substantial, but progress has been hampered by the challenges of identifying tumor-specific antigens. In current experiments, CD93 was identified as a novel target in AML that may have particular relevance in MLLr AML (Iwasaki et al., Cell Stem Cell. 2015; 17(4):412-421). Described herein are potent antigen-specific, anti-leukemic activity of CD93-CAR T cells, with mitigated on-target, off-tumor toxicities. CD93-CAR T cells spare HSPCs, a property distinct from many other AML CARs (Gill et al., Blood. 2014; 123(15):2343-2354; Kenderian et al., Leukemia. 2015; 29(8):1637-1647; Drent et al., Haematologica. 2016; 101(5):616-625; Laborda et al., Int J Mol Sci. 2017; 18(11); Tashiro et al., Mol Ther. 2017; 25(9):2202-2213; Jetani et al., Leukemia. 2018; 32(5):1168-1179), but may eliminate mature myeloid cells given CD93 expression on monocytes and neutrophils. The potential for prolonged neutropenia could have serious consequences for patients, and may preclude use of CD93-CAR T cells without a CAR T elimination strategy or as a lead in to HCT. No significant differences were found in most analyses comparing CD93-28z to CD93-BBz. However, if CAR T cells for AML are used as a bridge to HCT, a CD28 costimulatory domain may likely be desirable since they generally expand faster and do not persist as long as those with a 4-1BB costimulatory domain (Zhao et al., Structural Design of Engineered Costimulation Determines Tumor Rejection Kinetics and Persistence of CAR T Cells. Cancer Cell. 2015; 28(4):415-428). This “fast-on, fast-off” strategy could permit stem cell engraftment and hematologic recovery without the need for a CAR ablation strategy. However, further study is needed to understand the balance in AML patients between immediate CAR T cell efficacy for leukemic clearance and long-term CAR T persistence for durable leukemic control.

In physiologic and pathologic conditions, blood vessels and blood cells are inextricably linked from early in development. Endothelial and hematopoietic cells share a common precursor, termed a “hemangioblast” (Tavian et al., Aorta-associated CD34+ hematopoietic cells in the early human embryo. Blood 1996; 87(1):67-72; Bailey et al., Transplanted adult hematopoietic stems cells differentiate into functional endothelial cells. Blood 2004; 103(1):13-19; Choi et al., A common precursor for hematopoietic and endothelial cells. Development 1998; 125(4):725-732). Moreover, HSCs appear to promote migration and maintenance of healthy endothelial cells (Takakura et al. A role for hematopoietic stem cells in promoting angiogenesis. Cell 2000; 102(2):199-209), and endothelial cells secrete signals that increase survival and proliferation of both normal and malignant myeloid cells (Fiedler et al., Vascular endothelial growth factor, a possible paracrine growth factor in human acute myeloid leukemia. Blood 1997; 89(6):1870-1875; Ding et al., Endothelial and perivascular cells maintain haematopoietic stem cells. Nature 2012; 481(7382):457-462). Emerging data support a developmental trajectory of endothelium to hemogenic endothelium to HSCs and other myeloid progenitors, with significant overlap in essentially a continuum of gene expression (Guibentif et al., Single-Cell Analysis Identifies Distinct Stages of Human Endothelial-to-Hematopoietic Transition. Cell Rep. 2017; 19(1):10-19). Thus, the targets developed for AML may likely be co-expressed on endothelium, which are shown here for CD93. The results also raise concerns about susceptibility of endothelial cells to CAR T cells targeting CD123 and CD38, particularly in the setting of effective CARs that promote a pro-inflammatory microenvironment that could increase cell surface expression.

With the exception of the serious adverse events described in the UCART123 trial (Cellectis Reports Clinical Hold of UCART123 Studies [press release]), 2017), other clinical reports of CD123 CARs have not raised serious safety issues, but this could be falsely reassuring in the absence of reliable potency (Budde et al., Remissions of Acute Myeloid Leukemia and Blastic Plasmacytoid Dendritic Cell Neoplasm Following Treatment with CD123-Specific CAR T Cells: A First-in-Human Clinical Trial. American Society for Hematology 59th Annual Congress. 2017; 130(811); Cummins et al., Treating Relapsed/Refractory (RR) AML with Biodegradable Anti-CD123 CAR Modified T Cells. American Society for Hematology 59th Annual Congress. 2017; 130:1359). A patient with relapsed B-ALL received CD38 CART cells that resulted in multi-system organ damage refractory to supportive therapy and was highly concerning for on-target, off-tumor effects (Guo et al., Efficiency and side effects of anti-CD38 CAR T cells in an adult patient with relapsed B-ALL after failure of bi-specific CD19/CD22 CAR T cell treatment. Cell Mol Immunol. 2020; 17(4):430-432). As more patients are treated with CD123 and CD38 CARs and anti-leukemic activity improves, patients may need to be monitored for signs of capillary leak syndrome or other endothelial cell toxicity. These reports encourage caution and mandate detailed characterization of on-target, off-tumor toxicity early in CAR development.

Preclinical model systems including in vivo xenografts are insufficient for analysis of on-target, off-tumor toxicity, particularly when the scFv of the CAR does not cross-react with murine tissue, as is the case with the CD93-CAR. Publicly available databases that rely on bulk RNA sequencing can be useful for initial screening but may not identify a minority population of susceptible cells. High dimensional scRNA-seq was recently used to identify a rare population of CD19-expressing mural cells in the brain, to contribute to neurotoxicity seen with CD19 CAR (Parker et al. Single-Cell Analyses Identify Brain Mural Cells Expressing CD19 as Potential Off-Tumor Targets for CAR-T Immunotherapies. Cell. 2020.). Similarly, despite low expression on bulk parenchymal tissue, CD93 and CD123 can be clearly identified on endothelial populations. Development of CAR T cells against any novel tumor target requires thorough in silico screening of normal tissues that may have potential for on-target, off-tumor toxicities. This study demonstrates the power of integrating nuanced information provided by scRNAseq databases as part of these early analyses. However, even thorough analysis of transcriptional data cannot substitute for empiric confirmation of cell surface expression intensity and stability, and demonstration of CAR T cell safety in relevant model systems. Finally, as a general principle, murine models have not been highly predictive of immune toxicity observed in human studies, emphasizing the importance of carefully controlled early phase clinical trials for any novel immunotherapeutic agent.

Combinatorial targeting with the goal of circumventing CAR T cell on-target, off-tumor toxicity against vital normal tissues is an active area of investigation, and has been most highly emphasized for the development of CARs targeting solid tumors. The work presented here identifies the importance of developing logic gates for AML as well (Labanieh et al., Programming CAR-T cells to kill cancer. Nat Biomed Eng. 2018; 2(6):377-391). Autonomous, Boolean logic gate engineering approaches to date have largely focused on AND-gated CARs, which require two antigens to be present on target cells for effective cytotoxicity. Multiple AND-gated CARs have shown promise in preclinical models of AML (He et al., Bispecific and split CAR T cells targeting CD13 and TIM3 eradicate acute myeloid leukemia. Blood 2020; 135(10):713-723; Kugler et al., A recombinant tri-specific single-chain Fv derivative directed against CD123 and CD33 mediates effective elimination of acute myeloid leukaemia cells by dual targeting. Br J Haematol. 2010; 150(5):574-586) and other tumors (Kloss et al., Combinatorial antigen recognition with balanced signaling promotes selective tumor eradication by engineered T cells. Nat Biotechnol. 2013; 31(1):71-75; Lanitis et al., Chimeric antigen receptor T Cells with dissociated signaling domains exhibit focused antitumor activity with reduced potential for toxicity in vivo. Cancer Immunol Res. 2013; 1(1):43-53; Chen et al., Development of T cells carrying two complementary chimeric antigen receptors against glypican-3 and asialoglycoprotein receptor 1 for the treatment of hepatocellular carcinoma. Cancer Immunol Immunother. 2017; 66(4):475-489; Roybal et al., Precision Tumor Recognition by T Cells With Combinatorial Antigen-Sensing Circuits. Cell 2016; 164(4):770-779). NOT-gated CARs represent an alternative strategy that may prove effective when a particular tissue is the source of shared antigens, such as endothelial cell and myeloid cells as discussed here. NOT-gates target antigens selectively expressed on the cross-reactive tissue to propagate an inhibitory signal that interferes with the CAR T cell activation signal (Fedorov et al., Sci Transl Med. 2013; 5(215):215ra172). Although several groups have proposed NOT-gates as a solution to on-target, off-tumor toxicity of CAR T cells, thus far studies have been largely limited by lack of testing in tissue-specific model systems and by minimal optimization of these platforms (Fedorov et al., 2013; Dannenfelser et al., Discriminatory Power of Combinatorial Antigen Recognition in Cancer T Cell Therapies. Cell Syst. 2020; 11(3):215-228 e215; Williams et al., Precise T cell recognition programs designed by transcriptionally linking multiple receptors. Science 2020; 370(6520):1099-1104; Hamburger et al., Engineered T cells directed at tumors with defined allelic loss (see Mol Immunol. 2020; 128:298-310); Chen et al. De novo design of protein logic gates. Science 2020; 368(6486):78-84). Proof-of-principle is provided herein in a relevant in vitro model system that iCAR-based NOT-gated CAR T cells can circumvent CD93-CAR mediated endothelial cell toxicity. While further characterization and optimization of this platform is necessary, a successful NOT-gated strategy has the potential to expand the breadth of target antigens available for CAR T cell development and clinical translation.

In addition to other well-defined parameters including tumor heterogeneity, leukemic stem cell expression, and potential for hematopoietic cell toxicity (Haubner et al., Coexpression profile of leukemic stem cell markers for combinatorial targeted therapy in AML. Leukemia 2019; 33(1):64-74; Perna et al., Integrating Proteomics and Transcriptomics for Systematic Combinatorial Chimeric Antigen Receptor Therapy of AML. Cancer Cell. 2017; 32(4):506-519 e505), the RNA-seq dataset being generated could enable antigen selection for single and combinatorial, logic-gated AML CARs with a particular focus on avoiding endothelial cell reactivity. For CAR T cell therapy to become a more viable and widespread option in AML, development of CARs targeting cell surface proteins that have shared expression with some normal tissue seems unavoidable. Endothelial cell expression of AML targets is a particularly important consideration that requires more study and should be assessed for any new AML target.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A composition comprising a recombinant cell expressing a first chimeric antigen receptor (CAR) polypeptide and a second chimeric antigen receptor (CAR) polypeptide,
wherein the first CAR polypeptide comprises:
1a) a first extracellular ligand-binding domain having a binding affinity for a first ligand;
1b) a first transmembrane domain; and
1c) a first intracellular signaling domain,
wherein the second CAR polypeptide comprises:
2a) a second extracellular ligand-binding domain having a binding affinity for a second ligand;
2b) a second transmembrane domain; and
2c) a second intracellular signaling domain,
wherein binding of the first ligand to the first extracellular ligand-binding domain activates the first CAR polypeptide, and leads to activation of the recombinant cell to inhibit growth or kill a first cancer cell expressing a sufficient amount of the first ligand but not the second ligand, and
wherein binding of the second ligand to the second extracellular ligand-binding domain activates the second CAR polypeptide, and leads to reduction of the activation of the first CAR polypeptide, thereby reducing the capability of the recombinant cell to inhibit growth or kill a second cell expressing a sufficient amount of the second ligand.

2. The composition of claim 1, wherein the first ligand is expressed on the cell membrane of the first cancer cell and/or the second ligand is expressed on the cell membrane of the second cell.

3. The composition of claim 1 or 2, wherein the first ligand comprises CD93 or HER2.

4. The composition of any one of claims 1 to 3, wherein at least one of the first extracellular ligand-binding domain and the second extracellular ligand-binding domain comprises a ligand-binding domain of an antibody, an antigen-binding fragment, an antibody mimetic, or a receptor.

5. The composition of claim 4, wherein the antibody or the antigen-binding fragment is selected from the group consisting of a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a V_{H} domain, a V_{L} domain, an Fv fragment, a VNAR domain, and a V_{HH} domain.

6. The composition of any one of claims 1 to 5, wherein the first extracellular ligand-binding domain comprises an antibody, an antigen-binding fragment, or an antibody mimetic capable of binding to CD93, and wherein the first extracellular ligand-binding domain comprises a variable heavy (V_{H}) domain comprising CDR H1, CDR H2 and CDR H3 sequences in a V_{H} framework.

7. The composition of claim 6, wherein at least one of the CDR H1, CDR H2 and CDR H3 sequences comprises an amino acid sequence having at least 70% identity to SEQ ID NOs: 3, 4, or 5.

8. The composition of claim 6 or 7, wherein the variable heavy (V_{H}) domain comprises an amino acid sequence having at least 70% identity to SEQ ID NO: 1 or 2.

9. The composition of any one of claims 6 to 8, wherein the first extracellular ligand-binding domain further comprises a variable light (V_{L}) domain comprising CDR L1, CDR L2 and CDR L3 sequences in a V_{L} framework.

10. The composition of claim 9, wherein at least one of the CDR L1, CDR L2 and CDR L3 sequences comprises an amino acid sequence having at least 70% identity to SEQ ID NOs: 8, 9, or 10.

11. The composition of claim 9 or 10, wherein the variable light (V_{L}) domain comprises an amino acid sequence having at least 70% identity to SEQ ID NO: 6 or 7.

12. The composition of any one of claims 9 to 11, wherein the V_{H} and the V_{L} domains are conjugated through a linker.

13. The composition of claim 12, wherein the linker comprises a (G₄S)₃ linker.

14. The composition of any one of claims 6 to 13, wherein the first extracellular ligand-binding domain comprises an amino acid sequence having at least 70% identity to any one of SEQ ID NOs: 12-15.

15. The composition of any one of claims 1 to 14, wherein at least one of the first transmembrane domain and the second transmembrane domain comprises a transmembrane domain of or derived from CD8 or CD28.

16. The composition of any one of claims 1 to 15, wherein the first intracellular signaling domain comprises an intracellular signaling domain of or derived from CD3ζ.

17. The composition of any one of claims 1 to 16, wherein the second intracellular signaling domain comprises an immunoreceptor tyrosine-based inhibitory motif (ITIM) or ITIM-like domain.

18. The composition of claim 17, wherein the second intracellular signaling domain comprises an intracellular signaling domain of or derived from PD-1, CTLA-4, LAIR1 (LIR-1), TIGIT, Sig 2, Sig 5, Sig 6, Sig 10, BTLA, LAG3, CD300a, or SIRPα.

19. The composition of any one of claims 1 to 18, wherein at least one of the first CAR polypeptide and the second CAR polypeptide further comprises a hinge domain and/or a costimulatory domain.

20. The composition of claim 19, wherein the hinge domain is or is derived from an IgG₄ hinge domain, a CD28 hinge domain, or a CD8 hinge domain.

21. The composition of any one of claims 1 to 20, wherein the first cancer cell is derived from a cancer selected from Acute Myeloid Leukemia (AML), Myelodysplastic Syndrome, Myeloproliferative Neoplasms, and Acute Myeloid Leukemia with MLL rearrangements.

22. The composition of claim 21, wherein the cancer is acute myeloid leukemia (AML).

23. The composition of any one of claims 1 to 22, wherein the second cell is a non-cancer cell.

24. The composition of claim 23, wherein the non-cancer cell is an endothelial cell.

25. The composition of any one of claims 1 to 24, wherein the recombinant cell is an immune cell.

26. The composition of claim 25, wherein the immune cell is a T cell, a tumor-infiltrating lymphocyte (TIL), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell.

27. The composition of any one of claims 1 to 26, wherein activation of the first CAR polypeptide in response to the first antigen enhances cell proliferation, differentiation, cytokine production and/or cytotoxicity of the recombinant cell.

28. The composition of any one of claims 1 to 27, wherein activation of the second CAR polypeptide in response to the second antigen reduces cell proliferation, differentiation, cytokine production and/or cytotoxicity of the recombinant cell.

29. The composition of any one of claims 1 to 28, capable of:
i) inhibiting growth and/or killing the first cancer cell but not the second cell; and/or
ii) treating a cancer in a subject in need of.

30. A composition comprising an inhibitory chimeric antigen receptor (iCAR) polypeptide capable of reducing activation of an activating chimeric antigen receptor (aCAR) polypeptide,
wherein the aCAR polypeptide comprises:
1a) a first extracellular ligand-binding domain having a binding affinity for a first ligand;
1b) a first transmembrane domain; and
1c) a first intracellular signaling domain,
wherein the iCAR polypeptide comprises:
2a) a second extracellular ligand-binding domain having a binding affinity for a second ligand;
2b) a second transmembrane domain; and
2c) a second intracellular signaling domain,
wherein binding of the first ligand to the first extracellular ligand-binding domain activates the aCAR polypeptide, and leads to activation of a recombinant cell expressing both the aCAR and the iCAR polypeptides to inhibit growth or kill a first cancer cell expressing a sufficient amount of the first ligand but not the second ligand, and
wherein binding of the second ligand to the second extracellular ligand-binding domain activates the iCAR polypeptide, and leads to reduction of the activation of the aCAR polypeptide, thereby reducing the capability of the recombinant cell to inhibit growth or kill a second cell expressing a sufficient amount of the second ligand.

31. The composition of claim 30, wherein the first ligand is expressed on cell membrane of the first cancer cell and the second ligand is expressed on cell membrane of the second cell.

32. The composition of claim 30 or 31, wherein the first ligand comprises CD93 or HER2.

33. The composition of any one of claims 30 to 32, wherein at least one of the first extracellular ligand-binding domain and the second extracellular ligand-binding domain comprises a ligand-binding domain of an antibody, an antigen-binding fragment, an antibody mimetic, or a receptor.

34. The composition of claim 33, wherein the antibody or the antigen-binding fragment is selected from the group consisting of a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a V_{H} domain, a V_{L} domain, an Fv fragment, a VNAR domain, and a V_{HH} domain.

35. The composition of any one of claims 30 to 34, wherein the first extracellular ligand-binding domain comprises an antibody, an antigen-binding fragment, or an antibody mimetic capable of binding to CD93, and wherein the first extracellular ligand-binding domain comprises a variable heavy (V_{H}) domain comprising CDR H1, CDR H2 and CDR H3 sequences in a V_{H} framework.

36. The composition of claim 35, wherein at least one of the CDR H1, CDR H2 and CDR H3 sequences comprises an amino acid sequence having at least 70% identity to SEQ ID NOs: 3, 4, or 5.

37. The composition of claim 35 or 36, wherein the variable heavy (V_{H}) domain comprises an amino acid sequence having at least 70% identity to SEQ ID NO: 1 or 2.

38. The composition of any one of claims 35 to 37, wherein the first extracellular ligand-binding domain further comprises a variable light (V_{L}) domain comprising CDR L1, CDR L2 and CDR L3 sequences in a V_{L} framework.

39. The composition of claim 38, wherein at least one of the CDR L1, CDR L2 and CDR L3 sequences comprises an amino acid sequence having at least 70% identity to SEQ ID NOs: 8, 9, or 10.

40. The composition of claim 38 or 39, wherein the variable light (V_{L}) domain comprises an amino acid sequence having at least 70% identity to SEQ ID NO: 6 or 7.

41. The composition of any one of claims 38 to 40, wherein the V_{H} and the V_{L} domains are conjugated through a linker.

42. The composition of claim 41, wherein the linker comprises a (G₄S)₃ linker.

43. The composition of any one of claims 35 to 42, wherein the first extracellular ligand-binding domain comprises an amino acid sequence having at least 70% identity to any one of SEQ ID NOs: 12-15.

44. The composition of any one of claims 30 to 43, wherein at least one of the first transmembrane domain and the second transmembrane domain comprises a transmembrane domain of or derived from CD8 or CD28.

45. The composition of any one of claims 30 to 44, wherein the first intracellular signaling domain comprises an intracellular signaling domain of or derived from CD3ζ.

46. The composition of any one of claims 30 to 45, wherein the second intracellular signaling domain comprises an immunoreceptor tyrosine-based inhibitory motif (ITIM) or ITIM-like domain.

47. The composition of claim 46, wherein the second intracellular signaling domain comprises an intracellular signaling domain of or derived from PD-1, CTLA-4, LAIR1 (LIR-1), TIGIT, Sig 2, Sig 5, Sig 6, Sig 10, BTLA, LAG3, CD300a, or SIRPα.

48. The composition of any one of claims 30 to 47, wherein at least one of the aCAR polypeptide and the iCAR polypeptide further comprises a hinge domain and/or a costimulatory domain.

49. The composition of claim 48, wherein the hinge domain is or is derived from an IgG₄ hinge domain, a CD28 hinge domain, or a CD8 hinge domain.

50. The composition of any one of claims 30 to 49, wherein the first cancer cell is derived from a cancer selected from Acute Myeloid Leukemia (AML), Myelodysplastic Syndrome, Myeloproliferative Neoplasms, or Acute Myeloid Leukemia with MLL rearrangements.

51. The composition of claim 50, wherein the cancer is acute myeloid leukemia (AML).

52. The composition of any one of claims 30 to 51, wherein the second cell is a non-cancer cell.

53. The composition of claim 52, wherein the non-cancer cell is an endothelial cell.

54. The composition of any one of claims 30 to 53, wherein the recombinant cell is an immune cell.

55. The composition of claim 54, wherein the immune cell is a T cell, a tumor-infiltrating lymphocyte (TIL), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell.

56. The composition of any one of claims 30 to 55, wherein activation of the aCAR polypeptide in response to the first antigen enhances cell proliferation, differentiation, cytokine production and/or cytotoxicity of the recombinant cell.

57. The composition of any one of claims 30 to 56, wherein activation of the iCAR polypeptide in response to the second antigen reduces cell proliferation, differentiation, cytokine production and/or cytotoxicity of the recombinant cell.

58. The composition of any one of claims 30 to 56, capable of:
i) inhibiting growth and/or killing the first cancer cell but not the second cell; and/or
ii) treating a cancer in a subject in need of.

59. A system comprising an activating chimeric antigen receptor (aCAR) polypeptide and an inhibitory chimeric antigen receptor (iCAR) polypeptide,
wherein the aCAR polypeptide comprises:
1a) a first extracellular ligand-binding domain having a binding affinity for a first ligand;
1b) a first transmembrane domain; and
1c) a first intracellular signaling domain,
wherein the iCAR polypeptide comprises:
2a) a second extracellular ligand-binding domain having a binding affinity for a second ligand;
2b) a second transmembrane domain; and
2c) a second intracellular signaling domain,
wherein binding of the first ligand to the first extracellular ligand-binding domain activates the aCAR polypeptide, and leads to activation of a recombinant cell expressing both the aCAR and the iCAR polypeptides to inhibit growth or kill a first cancer cell expressing a sufficient amount of the first ligand but not the second ligand, and
wherein binding of the second ligand to the second extracellular ligand-binding domain activates the iCAR polypeptide, and leads to reduction of the activation of the aCAR polypeptide, thereby reducing the capability of the recombinant cell to inhibit growth or kill a second cell expressing a sufficient amount of the second ligand.

60. A composition comprising a polynucleotide encoding the iCAR polypeptide in the composition of any one of claims 30 to 58.

61. A composition comprising a first polynucleotide encoding the aCAR polypeptide in the system of claim 59 and a second polynucleotide encoding the iCAR polypeptide in the system of claim 59.

62. A composition comprising an expression vector comprising a polynucleotide encoding the iCAR polypeptide in the composition of any one of claims 30 to 58.

63. A composition comprising a first expression vector comprising a first polynucleotide encoding the aCAR polypeptide in the system of claim 59, and a second expression vector expressing a second polynucleotide encoding the iCAR polypeptide in the composition of claim 59.

64. A composition comprising an expression vector comprising a first polynucleotide encoding the aCAR polypeptide in the system of claim 59 and a second polynucleotide encoding the iCAR polypeptide in the system of claim 59.

65. A method for production of the composition of any one of claims 1 to 29 or the composition of any one of claims 30 to 58 in a cell, comprising introducing the composition of any one of claims 60 to 64 into the cell, and inducing expression of the CAR polypeptides under a condition.

66. A method for selectively activating the recombinant cell in the composition of any one of claims 1 to 29, comprising contacting the recombinant cell with the first ligand but not the second ligand.

67. A method for selectively inhibiting growth and/or killing of a cancer cell, comprising contacting the composition of any one of claims 1 to 29 with the cancer cell, wherein the cancer cell expresses a sufficient amount of the first ligand but not the second ligand.

68. A method for selectively inhibiting growth and/or killing of a first cancer cell but not a second cell, comprising contacting the composition of any one of claims 1 to 29 with the first cancer cell and the second cell, wherein the first cancer cell expresses a sufficient amount of the first ligand but not the second ligand, and the second cell expresses a sufficient amount of the second ligand.

69. A method of treating a cancer in a subject in need of, comprising administering to the subject a pharmaceutically effective amount of the composition of any one of claims 1 to 29, wherein the first cancer cell is derived from the cancer.


SEQUENCE LISTING (15 sequences)

SEQ ID NO: 1 - protein, 113 aa, synthetic construct
EVQLQQSGPELVKPGASVKIPCKASGYTFTDYHMDWVKQSHGKSLEWIGDIDPYNGDTVFNQKFKGKATLTVDKSSSTAYMELRSLTSEDTAVYYCTRGGDYWGQGTTLTVSS

SEQ ID NO: 2 - protein, 113 aa, synthetic construct
QVQLVQSGAEVKKPGASVKVSCKASGYTFTDYHMDWVKQAPGQGLEWIGDIDPYNGDTVFNQKFKGKATMTRDTSISTAYMELSRLRSDDTAVYYCTRGGDYWGQGTLVTVSS

SEQ ID NO: 3 - protein, 5 aa, synthetic construct
DYHMD

SEQ ID NO: 4 - protein, 17 aa, synthetic construct
DIDPYNGDTVFNQKFKG

SEQ ID NO: 5 - protein, 4 aa, synthetic construct
GGDY

SEQ ID NO: 6 - protein, 112 aa, synthetic construct
DVVMTQTPLSLPVSLGDQASISCRSSQTLVHSNGNTYLHWYLQKPGQSPKLLIYKVSNRFSGVPDRFSGSGSGTDFTLKISRVEAEDLGVYFCSQSTHVPFTFGSGTKLEIK

SEQ ID NO: 7 - protein, 112 aa, synthetic construct
DIVMTQTPLSLSVTPGQPASISCRSSQTLVHSNGNTYLHWYLQKPGQPPQLLIYKVSNRFSGVPDRFSGSGSGTDFTLKISRVEAEDVGVYFCSQSTHVPFTFGQGTKLEIK

SEQ ID NO: 8 - protein, 16 aa, synthetic construct
RSSQTLVHSNGNTYLH

SEQ ID NO: 9 - protein, 7 aa, synthetic construct
KVSNRFS

SEQ ID NO: 10 - protein, 9 aa, synthetic construct
SQSTHVPFT

SEQ ID NO: 11 - protein, 15 aa, synthetic construct
GGGGSGGGGSGGGGS

SEQ ID NO: 12 - protein, 240 aa, synthetic construct
EVQLQQSGPELVKPGASVKIPCKASGYTFTDYHMDWVKQSHGKSLEWIGDIDPYNGDTVFNQKFKGKATLTVDKSSSTAYMELRSLTSEDTAVYYCTRGGDYWGQGTTLTVSSGGGGSGGGGSGGGGSDVVMTQTPLSLPVSLGDQASISCRSSQTLVHSNGNTYLHWYLQKPGQSPKLLIYKVSNRFSGVPDRFSGSGSGTDFTLKISRVEAEDLGVYFCSQSTHVPFTFGSGTKLEIK

SEQ ID NO: 13 - protein, 240 aa, synthetic construct
DVVMTQTPLSLPVSLGDQASISCRSSQTLVHSNGNTYLHWYLQKPGQSPKLLIYKVSNRFSGVPDRFSGSGSGTDFTLKISRVEAEDLGVYFCSQSTHVPFTFGSGTKLEIKGGGGSGGGGSGGGGSEVQLQQSGPELVKPGASVKIPCKASGYTFTDYHMDWVKQSHGKSLEWIGDIDPYNGDTVFNQKFKGKATLTVDKSSSTAYMELRSLTSEDTAVYYCTRGGDYWGQGTTLTVSS

SEQ ID NO: 14 - protein, 240 aa, synthetic construct
QVQLVQSGAEVKKPGASVKVSCKASGYTFTDYHMDWVKQAPGQGLEWIGDIDPYNGDTVFNQKFKGKATMTRDTSISTAYMELSRLRSDDTAVYYCTRGGDYWGQGTLVTVSSGGGGSGGGGSGGGGSDIVMTQTPLSLSVTPGQPASISCRSSQTLVHSNGNTYLHWYLQKPGQPPQLLIYKVSNRFSGVPDRFSGSGSGTDFTLKISRVEAEDVGVYFCSQSTHVPFTFGQGTKLEIK

SEQ ID NO: 15 - protein, 240 aa, synthetic construct
DIVMTQTPLSLSVTPGQPASISCRSSQTLVHSNGNTYLHWYLQKPGQPPQLLIYKVSNRFSGVPDRFSGSGSGTDFTLKISRVEAEDVGVYFCSQSTHVPFTFGQGTKLEIKGGGGSGGGGSGGGGSQVQLVQSGAEVKKPGASVKVSCKASGYTFTDYHMDWVKQAPGQGLEWIGDIDPYNGDTVFNQKFKGKATMTRDTSISTAYMELSRLRSDDTAVYYCTRGGDYWGQGTLVTVSS